(19) United States
(12) Patent Application Publication
(10) Pub. No.: US 2024/0124510 A1
(43) Pub. Date: Apr. 18, 2024

(54) Cyclic Glycoaminoacid Derivatives

(71) Applicant: TFCHEM, Val-De-Reuil (FR)

(72) Inventors: Géraldine Deliencourt Godefroy, Sainte-Adresse (FR); Jocelyne Legoedec, Sotteville-Les-Rouen (FR); Lénaïg Lopes, Petit-Quevilly (FR)

(73) Assignee: TFCHEM, Val-De-Reuil (FR)

(21) Appl. No.: 18/273,433

(22) PCT Filed: Jan. 20, 2022
(86) PCT No.: PCT/EP2022/051208

§ 371 Date: Jul. 20, 2023

(30) Foreign Application Priority Data
Jan. 20, 2021 (EP) 21305070.1

(51) Int. Cl. C07H 7/06; A61K 8/60; A61K 31/706; A61P 29/00; A61Q 17/04

(52) U.S. Cl. CPC C07H 7/06; A61K 8/60; A61K 31/706; A61P 29/00; A61Q 17/04

(57) ABSTRACT

The present invention relates to compounds of the following formula (I): as well as their preparation process; their use in cosmetic or dermatological applications, in particular for the treatment and/or prevention of skin aging, skin protection or skin regeneration; for skin plumping and/or skin volumizing and/or skin densifying, and/or wrinkle filling and/or skin or hair moisturising and/or skin or hair relipiding and/or stimulation of hair growth; for the treatment of dry skin and/or atopic dermatitis and/or eczema and/or psoriasis; for the treatment and/or prevention of a fibrosis disease (e.g. an excessive scar such as a keloid or hypertrophic scar) or for healing; or for the treatment of inflammation (e.g. chronic, low-grade inflammation); and their use as adjuvant for preservation and/or protection and/or regeneration of a biological material or a microorganism.

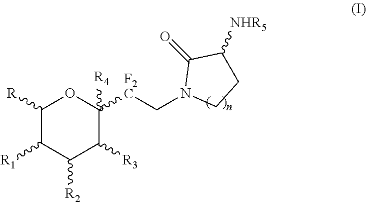

### TECHNICAL FIELD

The present invention relates to cyclic glycoaminoacid derivatives, as well as their preparation process; their use in cosmetic or dermatological applications, in particular for the treatment and/or prevention of skin aging, skin protection or skin regeneration; for skin plumping and/or skin volumizing and/or skin densifying, and/or wrinkle filling and/or skin or hair moisturising and/or skin or hair relipiding and/or stimulation of hair growth; for the treatment of dry skin and/or atopic dermatitis and/or eczema and/or psoriasis; for the treatment and/or prevention of a fibrosis disease (e.g. an excessive scar such as a keloid or hypertrophic scar) or for healing; or for the treatment of inflammation and especially chronic, low-grade inflammation, notably that develops in various aging tissues and referred as “inflammaging”, and their use as adjuvant for preservation and/or protection and/or regeneration of a biological material or a microorganism.

### BACKGROUND

Cutaneous aging is due by both intrinsic and extrinsic factors. Intrinsic aging is an inevitable chronological process that results in thin, dry skin, fine wrinkles, and gradual dermal atrophy. With aging, proliferation of cells in the basal layers reduces, epidermis become thinner and the contact area surface between dermis and epidermis decreases, resulting in a smaller exchange surface for nutrition supply to the epidermis and further weakened ability of basal cell proliferation. This process of decreased proliferative ability of cells includes keratinocytes, fibroblasts, and melanocytes. Extrinsic aging is due to the constantly exposure of the skin to stresses, including environmental (e.g. UV irradiation), chemicals (e.g. smoking), and nutritional stresses. Theses stresses affect the skin and lead to changes such as loss of elasticity, appearance of wrinkles and fine lines, dryness, itchiness, areas of hyperpigmentation, depigmented lesions, failure of the protective barrier function of the skin, and predisposition to skin cancer. Notably, long-term exposure to solar ultraviolet (UV) radiation is the primary factor of extrinsic skin aging and is referred to as photoaging.

Another feature of the aging process is a chronic progressive increase in the proinflammatory status, which was originally called “inflamm-aging”. Inflammaging refers to a continuous, low-grade inflammation associated with aging. Such chronic inflammatory response could build up with time and gradually cause tissue damage. It is considered as one of the driving forces for many age-related diseases and skin aging. This suggests a possible benefit of anti-inflammatory therapy in preventing age-related alteration in adipose tissue (as described in Metabolism. 2013 March; 62(3): 337-340).

Aging affects the different type of cells including adipocytes (as referred in Journal of Dermatology Science 71 (2013), 58-66), resulting in changes in facial contouring. This decrease of adiposity is due also to UV exposure and is also linked with an increased fibrosis. This process is related to an inflammatory response occurring with both aging and photoaging, and especially due to the production of inflammatory cytokines such as IL6. Fat loss, due to sun exposure as well as aging, involves at least two mechanisms: stimulation of lipolysis in mature adipocytes or inhibition of preadipocyte differentiation into mature adipocytes and lipogenesis.

The differentiation of preadipocytes requires a matrix remodeling necessary for the cells to differentiate and increase in size. Inflammation induces a decrease in remodeling capacity. It is thus clearly useful to avoid inflammation and to avoid fibrosis of adipose tissue in order to fight aging.

With age, the loss of skin elasticity and the degradation of adipose tissue lead to undesirable apparent effects on the body (hands, feet, buttocks, breasts, face) and notably on the face: appearance of lines and wrinkles, decrease of skin volume around the eyes and hollow cheeks. To reshape body, to fill expression lines and wrinkles, and to plump the skin, chirurgical fat injection (fat grafting or lipofilling) has been developed, and consists in restoring the volume of the skin, particularly the face, by the reinjection of fat removed from a rich fat site of the body. However, this technique currently used is expensive, can cause inflammatory reactions and needs to be redone several times for a satisfactory result. In order to find new lipofilling method, scientists were interested in skin physiology and more particularly in adipose tissue and its components. Adipose tissue is predominantly composed of adipocytes and of others cells such as preadipocytes, fibroblasts or endothelial cells. Adipocytes are the site of lipid synthesis and storage, they are provided from the process of adipogenesis also called adipocyte differentiation in which preadipocytes developed into mature adipocytes (Eur. J. Cell Biol. 2013, 92, 229-236). It has also been shown that fibroblasts and adipocytes are provided from common mesenchymal multipotent precursors (Exp. Dermatol. 2014, 23(9), 629-631). Thus, adipocyte cells could be generated by the differentiation of fibroblasts or from the stimulation of differentiation from preadipocytes.

Moreover, the stimulation of the adipogenesis and synthesis of lipid create an increase of adipocyte volume and therefore restore volume to the skin.

That is why, compounds with an efficacy to increase adipocytes number and volume have been described for their ability to act as skin plumping and/or skin volumizing and/or skin densifying and/or wrinkle filling and/or skin relipiding agents.

A compound, which is able to promote the growth (proliferation) of skin cell in particular under stress conditions, to protect them from different stresses and especially oxidative, to reduce fibrosis and inflammation, through the inhibition of cytokine release such as IL6, to promote matrix remodeling, to promote adipocytes lipogenesis, will thus be useful for treating and/or preventing skin aging, for skin plumping and for reducing wrinkles.

Aged skin, which is less plump than youthful skin, is characterized by decreased levels of hyaluronic acid (HA). HA is the simplest of the glycosaminoglycans, it is highly hygroscopic: hydrated hyaluronic acid can contain up to 1000-fold more water than its own weight. Hyaluronic acid was known for its regulatory activities with respect to epidermal proliferation and for its ability to retain water.

HA has been used in cosmetic formulations to treat wide ranges of skin problems including wrinkles, nasolabial folds, anti-aging, skin augmentation, skin hydration, and collagen stimulator. HA used to help the skin to hold and maintain elasticity, turgor and moisture and is claimed for its plumping effect (Dermato-endocrinology 2012, 43, 253-258).

That is why, compounds with an efficacy to increase hyaluronic acid synthesis have been described for their ability to act as skin plumping and/or skin volumizing and/or skin densifying and/or wrinkle filling and/or skin moisturising agents.

The changes which occur with aging within the adipose tissue and the preadipocytes and referred as inflammaging also has serious implication in obesity and different metabolic disorder such as insulin resistance and type 2 diabetes and heart disease.

A compound, which is able to reduce fibrosis and inflammation, through the inhibition of cytokine release such as IL6, in adipose tissue, will provide a good treatment for inflammaging alteration in adipose tissue especially in obesity, and in preventing the onset of various metabolic syndrome such as type 2 diabetes.

Stratum corneum lipids are required for the epidermal permeability barrier and for preventing the loss of water, so as to act as a barrier to prevent dehydration and/or to maintain hydration of the skin.

Lipids such as fatty acids and cholesterol are known to prevent and/or reduce skin dryness and wrinkles. Indeed, aging results in a decrease in epidermal cholesterol synthesis, which negatively affects permeability barrier homeostasis (as referred in Journal of Lipid Research 48 (2007), 20531-20546). The endogenous synthesis of lipid by skin cells such as keratinocytes could be a good alternative to treat the dry skin condition and the aging effect.

In addition, a decrease of lipid synthesis as well as inflammatory conditions can create skin barrier abnormalities observed in dry skin (WO98/10739), in atopic dermatitis, in eczema or in psoriasis (J. Invest. Dermatol. 1991, 96, 523-526; Contact Dermatitis 2008, 58, 255-262; Skin Pharmacol. Physiol. 2015, 28, 42-55).

Hyaluronic acid is a special moisturizing active ingredient, used in cosmetics, particularly formulated as emulsions or serums, claiming hydration. Because of the great number of polar groups present in its molecule, hyaluronic acid is a hydrophilic macromolecule with hydrating claims. In aqueous solutions it can form viscoelastic gels, and when it is applied to the skin it ensures moisturizing. Using cosmetic products such as creams or lotions that contain HA helps to moisturize the skin (molecules 2021, 26, 4429), but promoting the own production of hyaluronic acid by fibroblasts could be even a better approach by preventing the stability issue associated with the exogenous addition of HA.

Compounds which may alleviate inflammatory conditions, improve moisturizing, restore barrier function and restore lipid synthesis will provide a good treatment for dry skin, atopic dermatitis, eczema and psoriasis.

Moreover, it has also been proved that lipids and more particularly the cholesterol synthesis play a major role in hair biology. Thus, a decrease in lipid synthesis, and particularly in cholesterol, disturb hair cycle (J. Invest. Dermatol. 2010, 130(5), 1205-1207, J. Invest. Dermatol. 2010, 130, 1237-1248).

In addition, proliferation is the most widely tested marker of dermal papilla cells activity (International Journal of Cosmetic Science, 2018, 40, 429-450). Proliferation of dermal papilla cells determines their growth rate and mitotic index. Hence, the resultant effect on cell proliferation suggests the hair growth promoting effect. In addition, these cells in order to prevent apoptosis need to promote their growth and to be protected against inflammation and the production of cytokines.

Compounds, which may promote cells growth, alleviate inflammatory conditions, and restore lipid synthesis would be useful for stimulating hair growth.

Biological materials are often stored for a period of time prior to being used in vivo or in vitro, as well as microorganisms.

Storage conditions such as temperature and preservation media have significant effects on the quality of the biological material (or the microorganism) over a long period of time, while maintaining optimal cell growth and productivity, keeping them vital and functional without compromising their biological regenerative potential.

Many media and conditions have been developed in order to preserve cells, tissues and organs from plants, animals and humans. There is a need for media that protect cells from damage.

Compounds which may promote cells growth, protect cells from stress and especially oxidative stress would be good adjuvants in media for biological material or microorganism preservation.

During the classical wound healing process, three main complex steps are involved: 1) hemostasis/inflammation, 2) proliferation and 3) remodeling (BioMed Research International 2014, article 1D747584). First, the aggregation of platelets and the delivery of cytokines stop the haemorrhage and prevent infection (formation of a fibrin clot). Then, the proliferation of fibroblasts, the angiogenesis and the synthesis of extracellular matrix lead to the regeneration of dermal and epidermal tissue. And finally, the remodeling of the granulation tissue occurs.

Keloids and hypertrophic scars are the result of a dysfunction in the classical wound healing process following injury such as a surgical intervention, piercings, vaccination, acne, cuts, or burns. They consist of unaesthetic dense fibrous tissue that extends beyond the initial site of injury for the keloids or remain within the initial boundaries of injury for the hypertrophic scars.

Numerous treatments have been developed in order to treat, reduce and/or prevent keloids and hypertrophic scars such as conventional surgery, pressure therapy, topical silicone gel, radiation, laser, cryosurgery, injection of corticosteroids and chemical agents. Despite the large number of possible options to prevent and/or treat and/or attenuate keloids, none of them are really effective.

Compounds which are involved in extracellular matrix organization, in reducing fibrogenesis, in reducing the tensile strength of skin, in alleviating inflammatory conditions would provide a good treatment for wound healing process and especially keloid and hypertrophic scars.

CF₂-glycopeptide derivatives useful for the preservation of biological materials have been disclosed in WO2006/059227 and WO2007/125203. However, these compounds undergo stability issues and decompose on the very sensitive CF₂—C(═O)—NH function by releasing a strong difluorinated acid that tums to be highly cytotoxic.

Other CF₂-glycopeptide derivatives have been disclosed in WO2015/140178 for their preservative/protective effect on human skin fibroblasts and human nasal epithelial cells in vitro under different stresses, such as starvation conditions, UV stress, oxidative stress or bacterial stress, and in WO2018/138541 for their effect on the reduction of fibrosis through the down regulation of gene involved in extracellular matrix synthesis and the upregulation of gene involved in extracellular matrix degradation collagen expression in normal, aged, but also keloid fibroblasts.

### SUMMARY OF THE INVENTION

New cyclic CF₂-glycoaminoacid derivatives have been discovered that cumulate all the required properties for the above-mentioned cosmetic or dermatological applications, i.e. for anti-aging, skin protection or skin regeneration; for skin plumping and/or skin volumizing and/or skin densifying, and/or wrinkle filling and/or skin or hair moisturising and/or skin or hair relipiding and/or stimulation of hair growth; for the treatment of dry skin and/or atopic dermatitis and/or eczema and/or psoriasis; for the treatment and/or prevention of a fibrosis disease (e.g. an excessive scar such as a keloid or hypertrophic scar) or for healing; or for the treatment of inflammation and especially chronic, low-grade inflammation, notably that develops in various aging tissues and referred as “inflammaging”; and for their use as adjuvant for preservation and/or protection and/or regeneration of a biological material or a microorganism.

Said cyclic glycoaminoacid derivatives are able to promote the growth of skin cell, to protect them from different stresses and especially oxidative stress, to reduce fibrogenesis and inflammation, through the inhibition of cytokine release such as IL6, to promote matrix remodeling, to promote lipogenesis, to modify extracellular matrix organization, in reducing the tensile strength of skin, to promote the moisturisation as well as the plumping of the skin through the production of hyaluronic acid.

Compared to the CF₂-glycopeptide derivatives of the prior art disclosed in WO2015/140178 and in WO2018/138541, the cyclic glycoaminoacid derivatives according to the invention show unexpectedly a great efficacy at lower concentration.

The present invention relates to a compound of the following formula (I):

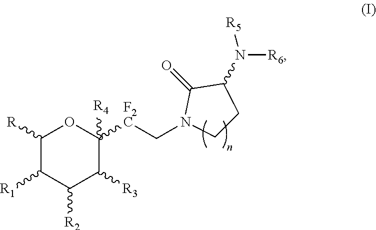

or a salt thereof, a solvate, a tautomer, a stereoisomer or a mixture of stereoisomers in any proportion, in particular a mixture of enantiomers, and particularly a racemate mixture, in which:
n represents 1 or 2, preferably 2,
R represents a hydrogen or fluorine atom or a CH₃, CH₂F, CH₂OSiR^{a1}R^{b1}R^{c1}, CH₂OR₈, CH₂OC(O)R₉, CH₂OCO₂R₁₀, CH₂OC(O)NR₁₁R₁₂, CH₂OP(O)(OR₁₃)₂ or CH₂OSO₃R₁₄ group,
R₁ and R₂ represent, independently from one another, a fluorine atom or an OSiR^{a2}R^{b2}R^{c2}, OR₁₅, OC(O)R₁₆, OCO₂R₁₇, OC(O)NR₁₈R₁₉, OP(O)(OR₂₀)₂ or OSO₃R₂₁ group,
R₃ represents a fluorine atom or an OSiR^{a3}R^{b3}R^{c3}, OR₂₂, OC(O)R₂₃, OCO₂R₂₄, OCONR₂₅R₂₆, OP(O)(OR₂₇)₂, OSO₃R₂₈, N₃₀, phthalimidyl, NR₂₉R₃₀, NR₃₁C(O)R₃₂, NR₃₃C(O)OR₃₄, N(C(O)R₃₅)C(O)R₃₆, N(C(O)R₃₇)C(O)OR₃₈ and N(C(O)OR₃₉)C(O)OR₄₀ group,
R₄ represents a hydrogen or halogen atom or an OSiR^{a4}R^{b4}R^{c4}, OR₄₁, OC(O)R₄₂, OCO₂R₄₃, OCONR₄₄R₄₅, OP(O)(OR₄₆)₂, or OSO₃R₄₇ group,
or R and R₁, together with the carbon atoms carrying them, form a cyclic acetal having the following formula:

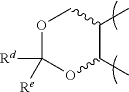

and/or (R₁ and R₂), (R₂ and R₃), and/or (R₃ and R₄), together with the carbon atoms carving them, form a cyclic acetal having the following formula:

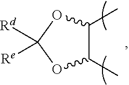

R₅ and R₆, identical or different, represent a hydrogen atom or a N-protecting group,
R₈, R₁₅, R₂₂ and R₄₁ represent, independently from one another, a hydrogen atom, a O-protecting group or a (C₁-C₆)alkyl, (C₂-C₆)alkenyl, (C₂-C₆)alkynyl, (C₃-C₇)cycloalkyl, 5- to 7-membered heterocycloalkyl, aryl, heteroaryl, aryl-(C₁-C₆)alkyl, heteroaryl-(C₁-C₆)alkyl, (C₁-C₆)-alkyl-aryl, (C₁-C₆)-alkyl-heteroaryl, saccharidic or polysaccharidic group, this group being possibly substituted with one or more groups chosen among a halogen atom, (C₁-C₆)alkoxy, OH, COOH and CHO; in particular a hydrogen atom, a (C₁-C₆)alkyl, aryl, aryl-(C₁-C₆)alkyl, saccharidic or polysaccharidic group, this group being possibly substituted with one or more groups chosen among a halogen atom, (C₁-C₆)alkoxy, OH, COOH and CHO; and more particularly a hydrogen atom, a (C₁-C₆)alkyl, aryl or aryl-(C₁-C₆)alkyl group, this group being possibly substituted with one or more groups chosen among a halogen atom, (C₁-C₆)alkoxy, OH, COOH and CHO,
R₉, R₁₀, R₁₆, R₁₇, R₂₃, R₂₄, R₃₂, R₃₄ to R₄₀, R₄₂ and R₄₃ represent, independently from one another, a (C₁-C₆)alkyl, (C₂-C₆)alkenyl, (C₂-C₆)alkynyl, (C₃-C₇)cycloalkyl, 5- to 7-membered heterocycloalkyl, aryl, heteroaryl, aryl-(C₁-C₆)alkyl, heteroaryl-(C₁-C₆)alkyl, (C₁-C₆)-alkyl-aryl or (C₁-C₆)-alkyl-heteroaryl group, this group being possibly substituted with one or more groups chosen among a halogen atom, (C₁-C₆)alkoxy, OH, COOH and CHO; and in particular a (C₁-C₆)alkyl, aryl or aryl-(C₁-C₆)alkyl group, this group being possibly substituted with one or more groups chosen among a halogen atom, (C₁-C₆)alkoxy, OH, COOH and CHO,
R₁₁, R₁₂, R₁₈, R₁₉, R₂₅, R₂₆, R₂₉ to R₃₁, R₃₃, R₄₄ and R₄₅ represent, independently from one another, a hydrogen atom or a (C₁-C₆)alkyl, (C₂-C₆)alkenyl, (C₂-C₆)alkynyl, aryl, heteroaryl, aryl-(C₁-C₆)alkyl, heteroaryl-(C₁-C₆)alkyl, (C₁-C₆)-alkyl-aryl or (C₁-C₆)-alkyl-heteroaryl group, this group being possibly substituted with one or more groups chosen among a halogen atom, (C₁-C₆)alkoxy, OH, COOH and CHO; advantageously a hydrogen atom or a (C₁-C₆)alkyl, (C₂-C₆)alkenyl, (C₂-C₆)alkynyl, aryl-(C₁-C₆)alkyl, heteroaryl-(C₁-C₆)alkyl group, this group being possibly substituted with one or more groups chosen among a halogen atom, (C₁-C₆)alkoxy, OH, COOH and CHO; and in particular a hydrogen atom or a (C₁-C₆)alkyl, aryl or aryl-(C₁-C₆)alkyl group, this group being possibly substituted with one or more groups chosen among a halogen atom, (C₁-C₆)alkoxy, OH, COOH and CHO,
R₁₃, R₁₄, R₂₀, R₂₁, R₂₇, R₂₈, R₄₆ and R₄₇ represent, independently from one another, a hydrogen atom or a (C₁-C₆)alkyl group,
R^{a1} to R^{a4}, R^{b1} to R^{b4} and R^{c1} to R^{c4} represent, independently from one another, a (C₁-C₆)alkyl, aryl or aryl-(C₁-C₆)alkyl group, and
R^{d} and R^{e} represent, independently from one another, a hydrogen atom or a (C₁-C₆)alkyl group.

The present invention relates also to a process for preparing a compound of formula (I) as defined above comprising the following steps:
(a) cyclizing a compound of the following formula (II):

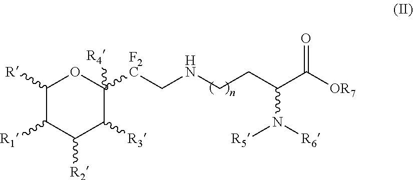

in which:
   n is as defined above,
   R′, R₁′, R₂′, R₃′, R₄′, R₅′ and R₆′ correspond respectively to R, R₁, R₂, R₃, R₄, R₅ and R₆ as defined above, optionally in a protected form, and
   R₇ represents a (C₁-C₆)alkyl (e.g. tert-butyl or methyl) or an aryl-(C₁-C₆)alkyl (e.g. benzyl), preferably a (C₁-C₆)alkyl (e.g. tert-butyl or methyl),
to obtain a compound of formula (I) optionally in a protected form,
(b) when R′, R₁′, R₂′, R₃′, R₄′, R₅′ and/or R₆′ represent a protected form of R, R₁, R₂, R₃, R₄, R₅ and/or R₆ respectively, deprotecting the protected form of R, R₁, R₂, R₃, R₄, R₅ and/or F₆ to obtain a compound of formula (I), and
(c) optionally salifying or solvating the compound obtained in previous step (a) or (b) to obtain a salt or solvate of a compound of formula (I).

The present invention relates also to a cosmetic or pharmaceutical (e.g. dermatological) composition comprising at least one compound of formula (I) as defined above and at least one physiologically acceptable excipient.

The present invention relates also a dressing comprising a pad, compress or sponge impregnated with a pharmaceutical composition according to the present invention as defined above.

The present invention relates also to a culture, storage and/or preservation medium comprising at least one compound of formula (I) as defined above.

The present invention relates also to the use, more particularly a cosmetic use, of a compound of formula (I) as defined above or a cosmetic or pharmaceutical (e.g. dermatological) composition according to the present invention as defined above for the treatment and/or prevention of skin aging, skin protection, or skin regeneration; or for skin plumping and/or skin volumizing and/or skin densifying and/or wrinkle filling and/or skin or hair moisturizing and/or skin or hair relipiding and/or stimulation of hair growth.

The present invention relates also to a compound of formula (I) as defined above or a cosmetic or pharmaceutical (e.g. dermatological) composition according to the present invention for use in the treatment and/or prevention of skin aging, skin protection or skin regeneration.

The present invention relates also to a compound of formula (I) as defined above or a cosmetic or pharmaceutical (e.g. dermatological) composition according to the present invention for use in the treatment of dry skin and/or atopic dermatitis and/or atopic eczema and/or psoriasis; for use in the treatment and/or prevention of a fibrosis disease, in particular an excessive scar such as a keloid or hypertrophic scar, or for use in healing; or for use in the treatment of inflammation and especially chronic, low-grade inflammation, notably that develops in various aging tissues and referred as “inflammaging”.

The present invention relates also to the use of a compound of formula (I) as defined above for the preservation and/or protection and/or regeneration of a biological material or a microorganism.

The present invention relates also to the use of a compound of formula (I) as defined above as an adjuvant in a culture, storage and/or preservation medium.

### Definitions

For the purpose of the invention, the term “physiologically acceptable” is intended to mean what is useful to the preparation of a cosmetic or pharmaceutical (e.g. dermatological) composition, and what is generally safe and non toxic, for a cosmetic or pharmaceutical (e.g. dermatological) use, i.e. in an animal, notably in a mammal such as a human.

By “topical” administration is meant in the framework of the present invention an administration on the skin or on mucous membranes (e.g. conjunctiva).

By “parenteral” administration is meant in the framework of the present invention an administration by injection, such as an intradermal or subcutaneous injection.

The term “physiologically acceptable salt and/or solvate” is intended to mean, in the framework of the present invention, a salt and/or solvate of a compound which is physiologically acceptable, as defined above, and which possesses the cosmetic or pharmacological activity of the corresponding compound.

In the context of the present invention, a “salt” is more particularly a “physiologically acceptable salt”. A salt or a physiologically acceptable salt can be:
(1) an acid addition salt formed with an inorganic acid such as hydrochloric, hydrobromic, sulfuric, nitric and phosphoric acid and the like; or formed with an organic acid such as acetic, benzenesulfonic, fumaric, glucoheptonic, gluconic, glutamic, glycolic, hydroxynaphtoic, 2-hydroxyethanesulfonic, lactic, maleic, malic, mandelic, methanesulfonic, muconic, 2-naphthalenesulfonic, propionic, succinic, dibenzoyl-L-tartaric, tartaric, p-toluenesulfonic, trimethylacetic and trifluoroacetic acid and the like, or
(2) a salt formed when an acid proton present in the compound is either replaced by a metal ion, such as an alkali metal ion, an alkaline-earth metal ion, or an aluminium ion; or coordinated with an organic or inorganic base. Acceptable organic bases comprise diethanolamine, ethanolamine, N-methylglucamine, triethanolamine, tromethamine and the like. Acceptable inorganic bases comprise aluminium hydroxide, calcium hydroxide, potassium hydroxide, sodium carbonate, sodium hydroxide and the like.

In the context of the present invention, a “solvate” is more particularly a “physiologically acceptable solvate”. Solvates of a cyclic glycoaminoacid derivative of the present invention or physiologically acceptable solvates of a cyclic glycoaminoacid derivative of the present invention include conventional solvates such as those formed during the last step of the preparation of the compounds of the invention due to the presence of solvents. As an example, mention may be made of solvates due to the presence of water (these solvates are also called hydrates) or ethanol.

For the purpose of this invention, “tautomer” is intended to designate the various tautomer forms that the sugar of compound (I) may assume, namely a pyranose (6-membered ring), furanose (5-membered ring) or linear (open form) form. However, for practical reasons, the sugar of compound (I) is represented in the present description by its pyranose form.

However, the compounds of the invention can assume various tautomer forms only when the radical R₄ represents an OH group, R₁ having also to represent an OH group in order that the compounds of the invention can be in the furanose form.

Thus, for example, in the galactose series, the compounds of the invention might appear under the following various forms, A representing the group

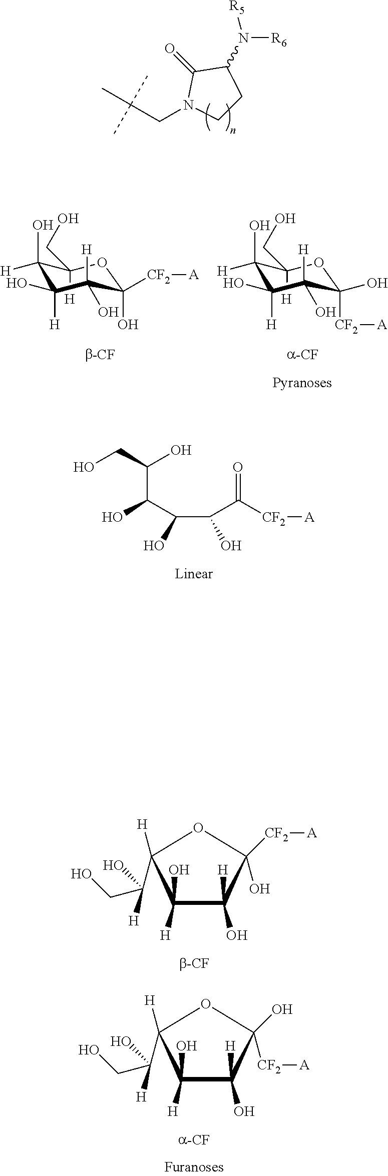

The group

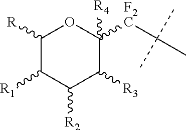

when R₄═R₁═OH can thus assume the following
pyranose form:

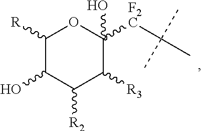

furanose form:

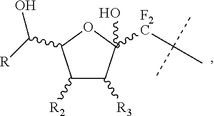

and
linear form:

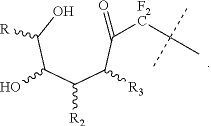

In the same way, the group

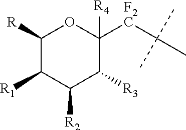

when R₄═R₁═OH can thus assume the following tautomer forms:
pyranose form:

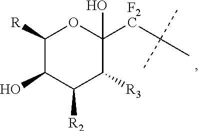

furanose form:

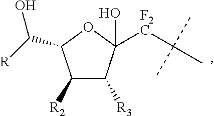

and
linear form:

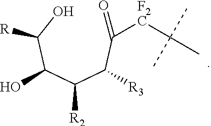

The anomeric carbon can appear in two different configurations in the closed pyranose and furanose forms.

The compounds of the invention can assume different tautomer forms which can be present in solution in equilibrium, with optionally a major tautomer form relatively to the other(s) tautomer form(s), or the compounds of the invention can assume only one tautomer form, such as only a pyranose form. This will depend notably on the nature of the medium, the temperature, the concentration of the compound, etc.

In this last case where the sugar assumes only one tautomer form, it is possible to block the configuration of the sugar in this tautomeric form when R₄═OH is transformed, notably by substitution of the OH group or conversion in a hydrogen or halogen atom.

Within the meaning of this invention, “stereoisomers” is intended to designate diastereoisomers or enantiomers. These are therefore optical isomers. Stereoisomers which are not mirror images of one another are thus designated as “diastereoisomers,” and stereoisomers which are non-superimposable mirror images are designated as “enantiomers”.

Notably, the sugar moiety and the amino acid moieties of the compounds of the invention can belong to the D or L series.

A carbon atom bond to four non-identical substituents is called a “chiral centre”.

An equimolar mixture of two enantiomers is called a racemate mixture.

The term “halogen” as used in the present invention refers to an atom of fluorine, bromine, chlorine or iodine. Advantageously, this is an atom of fluorine.

The term “(C₁-C₆)-alkyl” as used in the present invention refers to a saturated, linear or branched hydrocarbon chain comprising from 1 to 6 carbon atoms, in particular the methyl, ethyl, n-propyl, isopropyl, n-butyl, iso-butyl, sec-butyl, tert-butyl, n-pentyl, n-hexyl groups.

The term “(C₂-C₆)-alkenyl” as used in the present invention refers to a linear or branched hydrocarbon chain comprising at least one double bond and comprising from 2 to 6 carbon atoms, e.g., such as an ethenyl (vinyl) or propenyl (e.g. allyl) group.

The term “(C₂-C₆)-alkynyl” as used in the present invention refers to a linear or branched hydrocarbon chain comprising at least one triple bond and comprising from 2 to 6 carbon atoms, e.g., such as an ethynyl or propynyl group.

The term “(C₁-C₆)alkoxy”, as used in the present invention, refers to a (C₁-C₆)alkyl group as defined above bound to the molecule via an oxygen atom, including, but not limited to, methoxy, ethoxy, n-propoxy, iso-propoxy, n-butoxy, iso-butoxy, sec-butoxy, t-butoxy, n-pentoxy, n-hexoxy, and the like.

The term “(C₃-C₇)-cycloalkyl” as used in the present invention refers to a saturated hydrocarbon ring comprising from 3 to 7, advantageously from 5 to 7, carbon atoms, in particular the cyclohexyl, cyclopentyl or cycloheptyl group.

The term “heterocycloalkyl” as used in the present invention refers to a saturated hydrocarbon ring having 5 to 7 members, in which one or more, advantageously one or two, carbon atoms have been each replaced with a heteroatom, such as sulphur, nitrogen or oxygen atoms. It can be notably a tetrahydrofuranyl, piperidinyl, pyrrolidinyl, tetrahydropyranyl, or 1,3-dioxolanyl group.

The term “aryl”, as used in the present invention, refers to an aromatic hydrocarbon group comprising preferably 6 to 10 carbon atoms and comprising one or more fused rings, such as, for example, a phenyl or naphthyl group. Advantageously, it will be a phenyl group.

The term “heteroaryl” as used in the present invention refers to an aromatic group, preferably a 5- to 10-membered aromatic group, comprising one or more fused rings, in which the atoms of the ring(s) consist of one or more, advantageously 1 to 4, and more advantageously 1 or 2, heteroatoms, such as a nitrogen, oxygen or sulphur atom, the remainder being carbon atoms. A heteroaryl group can be notably a thienyl, furanyl, pyrrolyl, pyridyl, pyrimidyl, pyrazolyl, imidazolyl, tetrazolyl or indyl group.

The term “aryl-(C₁-C₆)-alkyl” as used in the present invention refers to any aryl group as defined above, which is bound to the molecule by means of a (C₁-C₆)-alkyl group as defined above. In particular, it can be a benzyl group.

The term “heteroaryl-(C₁-C₆)-alkyl” as used in the present invention refers to mean a heteroaryl group as defined above, which is bound to the molecule by means of a (C₁-C₆)-alkyl group as defined above.

The term “(C₁-C₆)-alkyl-aryl” as used in the present invention refers to a (C₁-C₆)-alkyl group as defined above, which is bound to the molecule by means of an aryl group as defined above. In particular, it can be a methylphenyl group.

The term “(C₁-C₆)-alkyl-heteroaryl” as used in the present invention refers to a (C₁-C₆)-alkyl group as defined above, which is bound to the molecule by means of a heteroaryl group as defined above.

The term “trialkylsilyl group”, as used in the present invention, refers to a group —SiAlk₁Alk₂Alk₃ in which Alk₁, Alk₂ and Alk₃, identical or different, represent a (C₁-C₆)-alkyl group as defined above. For example, it can be a trimethylsilyl or triethylsilyl group.

The term “protecting group”, as used in the present invention, refers to a chemical group which selectively blocks a reactive site in a multifunctional compound so as to allow selectively performing a chemical reaction on another unprotected reactive site.

The term “N-protecting group”, as used in the present invention, refers to those groups intended to protect an amine function against undesirable reactions during synthetic procedures. Commonly used N-protecting groups are disclosed in Greene, “Protective Groups In Organic Synthesis,” (John Wiley & Sons, New York (1981)). An amine function protected by a N-protecting group can be a carbamate, an amide, a sulfonamide, an N-alkyl derivative, an amino acetal derivative, a N-benzyl derivative, an imine derivative, an enamine derivative or a N-heteroatom derivative. In particular, N-protecting groups can be formyl; an aryl, such as a phenyl, optionally substituted with one or several methoxy groups such as p-methoxyphenyl (PMP); an aryl-(C₁-C₆)alkyl, such as a benzyl, the aryl moiety being optionally substituted with one or several methoxy groups, such as benzyl (Bn), p-methoxybenzyl (PMB) or 3,4-dimethoxybenzyl (DMPM); —CO—R_{GP1} such as acetyl (Ac), pivaloyl (Piv or Pv), benzoyl (Bz) or p-methoxybenzylcarbonyl (Moz); —CO₂—R_{GP1} such as tbutyloxycarbonyl (Boc), trichloroethoxycarbonyl (TROC), allyloxycarbonyl (Alloc), benzyloxycarbonyl (Cbz or Z) or 9-fluorenylmethyloxycarbonyl (Fmoc); —SO₂—R_{GP1} such as phenylsulfonyl, tosyl (Ts or Tos) or 2-nitrobenzenesulfonyl (also called nosyl—Nos or Ns); and the like, with R_{GP1} representing a (C₁-C₆)alkyl optionally substituted with one or several halogen atoms such as F or Cl; a (C₂-C₆)alkenyl such as an allyl; an aryl, such as a phenyl, optionally substituted with one or several groups chosen among OMe (methoxy) and NO₂ (nitro); an aryl-(C₁-C₆)alkyl, such as a benzyl, the aryl moiety being optionally substituted with one or several methoxy groups; or a 9-fluorenylmethyl group.

The N-protecting group can be in particular —CO₂—R_{GP1} such as Cbz, Boc or Fmoc, notably Cbz or Boc.

The term “O-Protecting group” as used in the present invention refers to a substituent which protects hydroxyl groups against undesirable reactions during synthetic procedures such as those O-protecting groups disclosed in Greene, “Protective Groups In Organic synthesis”, (John Wiley & Sons, New York (1981)). A hydroxyl group protected by a O-protecting group can be for example an ether, an ester, a carbonate, an acetal and the like. In particular, O-protecting groups can be a (C₁-C₆)alkyl optionally substituted with one or several (notably 1 to 3) halogen atoms (such as chlorine atoms), such as methyl, ethyl, tert-butyl or 2,2,2-trichloroethyl; an aryl-(C₁-C₆)alkyl, such as a benzyl, the aryl moiety being optionally substituted with one or several methoxy groups, such as benzyl (Bn) or p-methoxybenzyl (PMB); a trityl group of formula —CAr₁Ar₂Ar₃ such as triphenylmethyl (also called trityl—Tr), (4-methoxyphenyl)diphenylmethyl (also called methoxytrityl—NMT) or bis-(4-methoxyphenyl)phenylmethyl (also called dimethoxytrityl—DMT); a substituted methyl group of formula —CH₂OR_{GP2} or —CH₂SR_{GP2} (in particular —CH₂OR_{GP2}), for example, methoxymethyl (MOM), benzyloxymethyl, 2-methoxyethoxymethyl (MEM), 2-(trimethylsilyl)ethoxymethyl or methylthiomethyl; a substituted ethyl group of formula —CH₂CH₂OR_{GP2} or —CH₂CH₂SR_{GP2} (in particular —CH₂CH₂OR_{GP2}), for example, ethoxyethyl (EE); a silyl group of formula —SiR_{GP3}R_{GP4}R_{GP5}, for example, trimethylsilyl (TMS), triethylsilyl (TES), t-butyldimethylsilyl (TBS or TBDMS) and t-butyldiphenylsilyl (TBDPS); carbonylated groups of formula —CO—R_{GP6} such as acetyl (Ac), pivaloyl (Piv or Pv) or benzoyl (Bz) or of formula —CO₂—R_{GP7} such as allyloxycarbonyl (Alloc) or 9-fluorenylmethyloxycarbonyl (Fmoc); or a tetrahydropyranyl

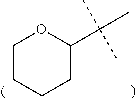

(THP) or tetrahydrofuranyl

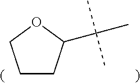

group; with Ar₁, Ar₂ and Arₙ representing, independently from one another, an aryl, such as a phenyl, optionally substituted with one or several methoxy groups; R_{GP2} representing a (C₁-C₆)alkyl (such as methyl or ethyl) optionally substituted with an aryl (such as phenyl), a (C₁-C₆)alkoxy (such as methoxy) or a trialkylsilyl group (such as SiMe₃); R_{GP3}, R_{GP4} and R_{GP5} representing, independently from one another, a (C₁-C₆)alkyl or aryl (such as phenyl) group; and R_{GP6} and R_{GP7} representing, independently of each other, a (C₁-C₆)alkyl, a (C₂-C₆)alkenyl, an aryl, an aryl-(C₁-C₆)alkyl or a 9-fluorenylmethyl group.

The O-protecting group can be in particular a (C₁-C₆)alkyl group or an aryl-(C₁-C₆)alkyl group, preferably an aryl-(C₁-C₆)alkyl group (such as a benzyl).

The term “saccharide” as used in the present invention refers to erythrose, threose, ribose, arabinose, xylose, lyxose, allose, altrose, glucose, mannose, gulose, idose, galactose, talose, erythrulose, ribulose, xylulose, psicose, fructose, sorbose or tagatose, in D or L form.

The term “saccharidic group” as used in the present invention refers to a saccharide as defined above bond to the molecule by means of its oxygen atom present at the anomeric centre.

The term “polysaccharide” as used in the present invention refers to a chain comprising at least 2, and preferably 2 to 10 saccharides as defined above bound together by means of an oxygen bridge formed between the OH function at the anomeric position of a saccharide and the OH function not at the anomeric position of another saccharide.

The term “polysaccharidic group” as used in the present invention refers to a polysaccharide as defined above bond to the molecule by means of the oxygen atom present at the anomeric centre of the terminal saccharide.

The term “leaving group” as used in the present invention refers to refers to a chemical group which can be easily replaced with a nucleophile during a nucleophile substitution reaction, the nucleophile being notably a primary amine. Such a leaving group can be in particular a halogen atom, a sulfonate, a N-succinimidyloxy, a 4-nitro-phenyloxy, pentafluorophenoxy or a N-benzotriazoloxy. The sulfonate is in particular a group —OSO₂—R_{LG} with R_{LG} representing a (C₁-C₆)alkyl, aryl, aryl-(C₁-C₆)alkyl or (C₁-C₆)alkyl-aryl group, the said group being optionally substituted with one or several halogen atoms such as fluorine atoms. The sulfonate can be notably a mesylate (CH₃—S(O₂)O—), a triflate (CF₃—S(O)₂O—) or a tosylate (p-Me-C₆H₄—S(O)₂O—).

The term “preservation” of a biological material or a microorganism as used in the present invention refers to the fact to maintain the state (notably the structure and function) of the biological material or the microorganism as it already exists or to prevent or limit the degradation of this state.

The term “protection” of a biological material or a microorganism as used in the present invention refers to the fact that the biological material or the microorganism is protected against an internal or external aggression, such as a stress, for ex. an oxidative stress (for ex. UV), a change of temperature, a change of pH, a chemical or bacterial contamination, starvation conditions, etc.

The term “regeneration” of a biological material or a microorganism as used in the present invention refers to the fact to recover the state (notably the structure and function) of the biological material or the microorganism as it existed before an internal or external aggression, such as a stress, for ex. an oxidative stress (for ex. UV), a change of temperature, a change of pH, a chemical or bacterial contamination, starvation conditions, etc. It concerns more particularly a biological material, such as cells.

The term “protection” of skin as used in the present invention refers to the fact to maintain the state (notably the structure and function) of the skin and cells of the skin as it already exists or to prevent or limit the degradation of this state by protecting them against an internal or external aggression, such as a stress, for ex. an oxidative stress (for ex. UV), a change of temperature, a change of pH, a chemical or bacterial contamination, denutrition conditions, etc.

The term “regeneration” of skin as used in the present invention refers to the fact to recover the state (notably the structure and function) of the skin and cells as it existed before an internal or external aggression, such as a stress, for ex. an oxidative stress (for ex. UV), a change of temperature, a change of pH, a chemical or bacterial contamination, denutrition conditions, etc.

The term “treatment and/or prevention of skin aging” as used in the present invention means to prevent, avoid or delay the onset of the signs of skin aging and/or to reduce or suppress the signs of skin aging. The signs of skin aging can be for example wrinkles, fine lines, skin atrophy, loss of elasticity, dryness, etc.

The terms “skin plumping”, “skin volumizing” and “skin densifying”, as used in the present invention, refers to the fact to reshape the skin and to increase volume of the skin, notably by increasing the adipose volume.

The term “wrinkle filling”, as used in the present invention, refers to the fact to restore the volume, fullness and smoothness of the skin in order to reduce or eliminate wrinkles, including expression lines, notably by increasing the adipose volume.

The term “skin or hair moisturising”, as used in the present invention, refers to the fact to increase the moisture content of the skin or the hair and to keep the skin soft, supple and smooth and to keep the hair soft, supple and shine, notably by increasing lipid (e.g. cholesterol) synthesis.

The term “skin or hair relipiding”, as used in the present invention, refers to the fact to increase the lipid content of the skin or the hair in order to restore the hydrolipidic film of the skin or the hair so as to keep the skin soft, supple and smooth and to keep the hair soft, supple and shine.

By “fibrosis disease” is meant in the present invention a disease involving the formation of excess fibrous connective tissue. When this formation of excess fibrous connective tissue occurs in response to injury (for ex. a surgical intervention, piercings, vaccination, acne, cuts, or burns), the fibrosis disease is called “excessive scar”. It can be keloids or hypertrophic scars. They consist of unaesthetic dense fibrous tissue that extends beyond the initial site of injury for the keloids or remain within the initial boundaries of injury for the hypertrophic scars.

By “treatment” of a disease is meant in the present invention the disappearance or the reduction of one or several (notably all) of the symptom(s) of the disease.

By “prevention” of a disease is meant in the present invention the fact to prevent or reduce the appearance of one or several (notably all) of the symptom(s) of the disease.

### DETAILED DESCRIPTION

Cyclic Glycoaminoacid Derivatives

The cyclic glycoaminoacid derivatives according to the invention are compounds of formula (I) as defined above.

The compound of formula (I) according to the invention can be for example a compound of the following formula (Ia) or (Ib):

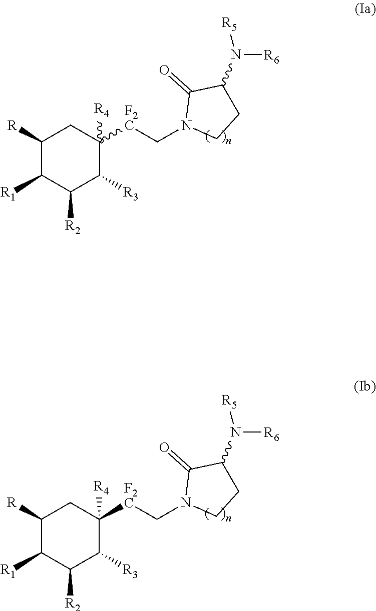

or a salt thereof, a solvate, a tautomer, a stereoisomer or a mixture of stereoisomers in any proportion, in particular a mixture of enantiomers, and particularly a racemate mixture, in which n, R, R₁, R₂, R₃, R₄, R₅ and R₆ are as defined above or below.

The compound of formula (I) according to the invention can be for example a compound of the following formula (Ic) or (Id):

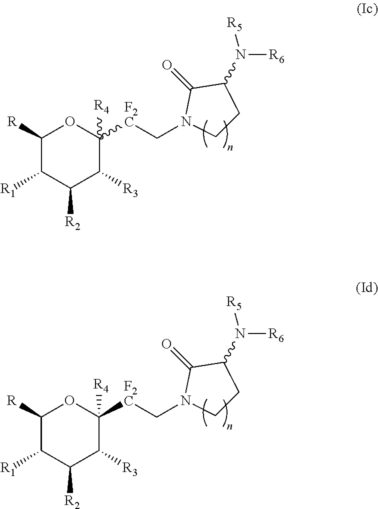

or a salt thereof, a solvate, a tautomer, a stereoisomer or a mixture of stereoisomers in any proportion, in particular a mixture of enantiomers, and particularly a racemate mixture, in which n, R, R₁, R₂, R₃, R₄, R₅ and R₆ are as defined above or below.

R can represent a CH₂OSiR^{a1}R^{b1}R^{c1}, CH₂OR₈, CH₂OC(O)R₉, CH₂OCO₂R₁₀, CH₂OC(O)NR₁₁R₁₂, CH₂OP(O)(OR₁₃)₂ or CH₂OSO₃R₁₄ group, advantageously a CH₂OSiR^{a1}R^{b1}R^{c1}, CH₂OR₈ or CH₂OC(O)R₉ group, more advantageously a CH₂OR₈ or CH₂OC(O)R₉ group, and even more advantageously a CH₂OR₈ group.

R can represent in particular a CH₂OR₈ group with R₈ representing a hydrogen atom, a O-protecting group or a (C₁-C₆)-alkyl, aryl or aryl-(C₁-C₆)-alkyl group; or a CH₂OC(O)R₉ group with R₉ representing a (C₁-C₆)-alkyl, aryl or aryl-(C₁-C₆)-alkyl group.

R can represent more particularly a CH₂OR₈ group with R₈ representing a hydrogen atom or a O-protecting group. For instance, R can represent a CH₂OH or CH₂OBn group.

R₁ and R₂ can represent, independently from one another, an OSiR^{a2}R^{b2}R^{c2}, OR₁₅, OC(O)R₁₆, OCO₂R₁₇ or OC(O)NR₁₈R₁₉ group, advantageously an OSiR^{a2}R^{b2}R^{c2}, OR₁₅ or OC(O)R₁₆ group, more advantageously an OR₁₅ or OC(O)R₁₆ group, and even more advantageously an OR₁₅ group.

R₁ and R₂ can represent in particular, independently from one another, an OR₁₅ group with R₁₅ representing a hydrogen atom, a O-protecting group or a (C₁-C₆)-alkyl, aryl or aryl-(C₁-C₆)-alkyl group; or an OC(O)R₁₆ group R₁₆ representing a (C₁-C₆)-alkyl, aryl or aryl-(C₁-C₆)-alkyl group.

R₁ and R₂ can represent more particularly, independently from one another, an OR₁₅ group with R₁₅ representing a hydrogen atom or a O-protecting group. For instance, R₁ and R₂ can represent an OH or OBn group.

Preferably, R₁ and R₂ are identical, and represent notably an OH or OBn group.

In particular, R represents a CH₂OR₈ group and R₁ and R₂ represent, independently from one another, an OR₁₅ group, R₈ and R₁₅ representing advantageously a hydrogen atom or an O-protecting group (for example Bn). R₈ and the two R₁₅ groups can be identical, such as H or an O-protecting group (for example Bn).

According to another particular embodiment, R═CH₂OH and R₁═R₂═OH or R═CH₂OBn and R₁═R₂=OBn.

According to a first embodiment, R₃ represent an OSiR^{a3}R^{b3}R^{c3}, OR₂₂, OC(O)R₂₃, OCO₂R₂₄, OCONR₂₅R₂₆, NR₂₉R₃₀, NR₃₁C(O)R₃₂, NR₃₃C(O)OR₃₄, N(C(O)R₃₅)C(O)R₃₆, N(C(O)R₃₇)C(O)OR₃₈ or N(C(O)OR₃₉)C(O)OR₄₀ group, advantageously an OSiR^{a3}R^{b3}R^{c3}. OR₂₂, OC(O)R₂₃, NR₂₉R₃₀, NR₃₁C(O)R₃₂ or NR₃₃C(O)OR₃₄ group, more advantageously an OR₂₂, OC(O)R₂₃ or NR₃₁C(O)R₃₂ group, and even more advantageously an OR₂₂ or NR₃₁C(O)R₃₂ group.

R₃ can represent in particular an OR₂₂ group with R₂₂ representing a hydrogen atom, a O-protecting group or a (C₁-C₆)-alkyl, aryl or aryl-(C₁-C₆)-alkyl group; an OC(O)R₂₃ group with R₂₃ representing a (C₁-C₆)-alkyl, aryl or aryl-(C₁-C₆)-alkyl group; or a NR₃₁C(O)R₃₂ group with R₃₁ representing a hydrogen atom or a (C₁-C₆)-alkyl, aryl or aryl-(C₁-C₆)-alkyl group and R₃₂ representing a (C₁-C₆)alkyl, aryl or aryl-(C₁-C₆)alkyl group.

R₃ can represent more particularly an OR₂₂ group with R₂₂ representing a hydrogen atom or a O-protecting group (for example Bn); or a NR₃₁C(O)R₃₂ group with R₃₁ representing a hydrogen atom and R₃₂ representing a (C₁-C₆)alkyl. For instance, R₃ can represent an OH, OBn, OMOM or NHAc group, in particular OH or OBn.

According to a second embodiment R₃ can represent an OSiR^{a3}R^{b3}R^{c3}, OR₂₂, OC(O)R₂₃, OCO₂R₂₄ or OCONR₂₅R₂₆ group, advantageously an OSiR^{a3}R^{b3}R^{c3}, OR₂₂ or OC(O)R₂₃ group, more advantageously an OR₂₂ or OC(O)R₂₃ group, and even more advantageously an OR₂₂ group.

R₃ can represent in particular an OR₂₂ group with R₂₂ representing a hydrogen atom, a O-protecting group or a (C₁-C₆)-alkyl, aryl or aryl-(C₁-C₆)-alkyl group; or an OC(O)R₂₃ group R₂₃ with representing a (C₁-C₆)-alkyl, aryl or aryl-(C₁-C₆)-alkyl group.

R₃ can represent more particularly an OR₂₂ group with R₂₂ representing a hydrogen atom or a O-protecting group (for example Bn). For instance, R₃ can represent an OH or OBn group.

According to a particular embodiment, R₁, R₂ and R₃ are identical.

According to another particular embodiment, R represents a CH₂OR₈ group; R₁ and R₂ represent, independently from one another, an OR₁₅ group; and R₃ represents an OR₂₂ group, R₈, R₁₅ and R₂₂ representing advantageously a hydrogen atom or an 0-protecting group (for example Bn). R₈ and the two R₁₅ groups can be identical, such as H or an O-protecting group (for example Bn). R₈, the two R₁₅ and R₂₂ groups can also be identical, such as H or an O-protecting group (for example Bn).

According to another particular embodiment, R═CH₂OH, R₁═R₂═OH or R₁═R₂=R₃═OH.

R₄ can advantageously represent a hydrogen or halogen atom or an OR₄₁ group; in particular a hydrogen atom or an OR₄₁ group; and more particularly an OR₄₁ group.

Yet even more advantageously, R₄ may represent a hydrogen or halogen atom or an OH, O-protecting, —O—(C₁-C₆)-alkyl, —O-aryl and —O—(C₁-C₆)-alkyl-aryl group; in particular, a hydrogen atom or an OH, O-protecting, —O—(C₁-C₆)-alkyl, —O-aryl and —O—(C₁-C₆)-alkyl-aryl group; and more particularly an OH, O-protecting, —O—(C₁-C₆)-alkyl, —O-aryl and —O—(C₁-C₆)-alkyl-aryl group.

R₄ can also represent a hydrogen or halogen atom or an OH, —O—(C₁-C₆)-alkyl, —O-aryl and —O—(C₁-C₆)-alkyl-aryl group; in particular, a hydrogen atom or an OH, —O—(C₁-C₆)-alkyl, —O-aryl and —O—(C₁-C₆)-alkyl-aryl group; and more particularly an OH, —O—(C₁-C₆)-alkyl, —O-aryl and —O—(C₁-C₆)-alkyl-aryl group.

In particular, R₄ can represent a hydrogen or halogen (such as Br, Cl, F) atom or an OH or O-protecting group (for ex. OMe or OBn); advantageously a hydrogen atom or an OH or O-protecting group (for ex. OMe or OBn); such as H or OH.

R₄ can be in particular an OH or O-protecting group such as OH, OMe or OBn; and preferably an OH group.

R₅ and R₆, identical or different, can advantageously represent a hydrogen atom or a N-protecting group being a —CO₂—R_{GP1} group with R_{GP1} as defined above, such as Cbz, Boc or Fmoc, notably Cbz or Boc. Preferably, at least one of R₅ and R₆ is a hydrogen atom. Most preferably, both R₅ and R₆ represent a hydrogen atom.

According to a particular embodiment, R═CH₂OH or CH₂OBn and R₁═R₂═R₃ ═OH or OBn.

According to another particular embodiment, R═CH₂OH and R₁═R₂=R₃═OH.

According to yet another particular embodiment, R═CH₂OH, R₁═R₂=R₃═OH and R₄═H or OH, in particular OH.

According to a particular embodiment, the compound of the invention is a compound of formula (I):

or a salt thereof, a solvate, a tautomer, a stereoisomer or a mixture of stereoisomers in any proportion, in particular a mixture of enantiomers, and particularly a racemate mixture, in which:
n represents 1 or 2, and preferably 2,
R represents CH₂OR₈,
R₁ and R₂ represent, independently from one another, OR₁₅,
R₃ represents OR₂₂,
R₄ represents H or OR₄₁, in particular OR₄₁,
or R and R₁, together with the carbon atoms carrying them, form a cyclic acetal having the following formula:

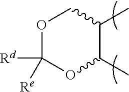

and/or (R₁ and R₂), (R₂ and R₃), and/or (R₃ and R₄), together with the carbon atoms carrying them, form a cyclic acetal having the following formula:

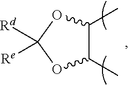

R₈, R₁₅ and R₂₂ represent, independently from one another, a hydrogen atom or a O-protecting group (for example a (C₁-C₆)alkyl or aryl-(C₁-C₆)alkyl group),
R₄₁ represents a hydrogen atom, a O-protecting group (for example a (C₁-C₆)alkyl or aryl-(C₁-C₆)alkyl group) or a (C₁-C₆)alkyl, aryl, aryl-(C₁-C₆)alkyl, or (C₁-C₆)-alkyl-aryl group, this group being possibly unsubstituted or substituted with one or more groups chosen among a halogen atom and (C₁-C₆)alkoxy, and
R^{d} and R^{e} represent, independently from one another, a hydrogen atom or a (C₁-C₆)alkyl group.

In this embodiment, R₅ and R₆, identical or different, can advantageously represent a hydrogen atom or a N-protecting group being a —CO₂—R_{GP1} group with R_{GP1} as defined above, such as Cbz, Boc or Fmoc, notably Cbz or Boc. Preferably, at least one of R₅ and R₆ is a hydrogen atom. Most preferably, both R₅ and R₆ represent a hydrogen atom.

The compound of formula (I) can be chosen among the following compounds:

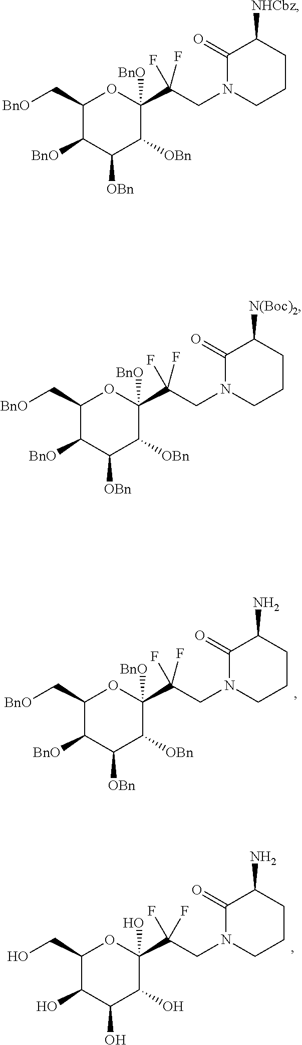

and the salts and solvates thereof (notably acid addition salts in particular with hydrochloric acid or acetic acid, more particularly with hydrochloric acid).

The compound of formula (I) can also be chosen among the following compounds:

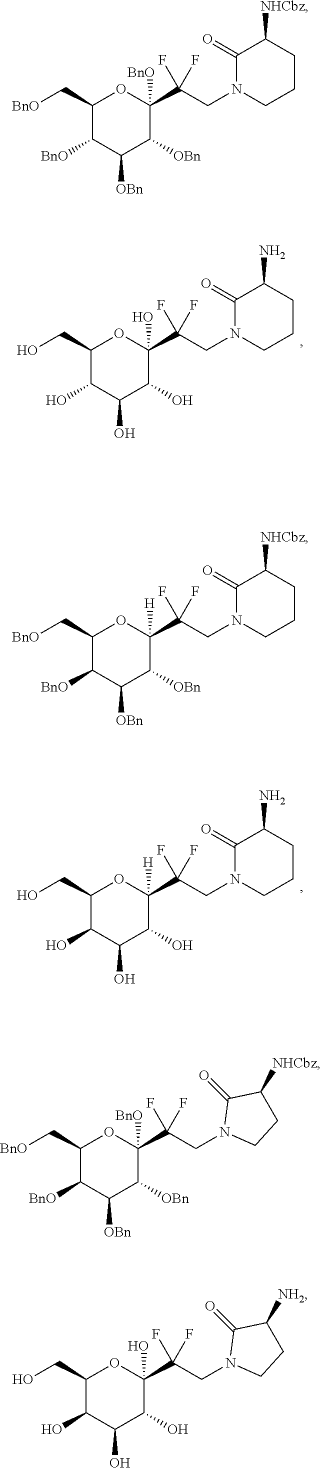

and the salts and solvates thereof (notably acid addition salts in particular with hydrochloric acid or acetic acid, more particularly with hydrochloric acid).

In particular, the compound of formula (I) can be compound 4, compound 5, compound 6, compound 15, compound 19, compound 22, compound 23, compound 24, compound 27, compound 28, compound 29, compound 32, compound 33 or compound 34 as described in the examples below.

Preferably, the compound of formula (I) is compound 6 or a salt and/or solvate thereof, such as an acid addition salt in particular with hydrochloric acid or acetic acid, such as with hydrochloric acid. Most preferably, it is compound 6.

Process of Preparation

The present invention relates also to a process for preparing a compound of formula (I) as defined above comprising steps (a) to (c).

Step (a):

The cyclisation step can be performed in an acidic medium, notably in the presence of an acid such as acetic acid on a compound of formula (II).

The reaction can be performed in a solvent such as toluene, notably at reflux.

In the case of this reaction, advantageously R₅′≠H and/or R₆′≠H, R′≠CH₂OH, R₁′≠OH, R₂′≠OH, R₃′≠OH, and R₄′≠OH. Thus, to prepare compounds which such substituents, the OH or NH₂ functions should be preferably protected by a protecting group as defined above before cyclising the compound of formula (II) into a compound of formula (I).

The compound of formula (II) can be prepared by reducing the imine function of a compound of the following formula (III):

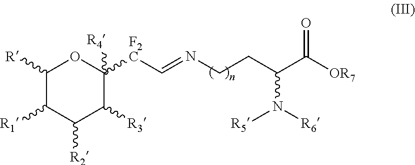

in which n, R′, R₁′, R₂′, R₃′, R₄′, R₅′, R₆′ and R₇ are as defined above.

The reduction reaction can be carried out in the presence of a borohydride such as NaBH₃CN or NaBH(OAc)₃.

The reaction can be carried out in a solvent such as dichloroethane.

The compound of formula (III) can be prepared by reacting a compound of the following formula (IV):

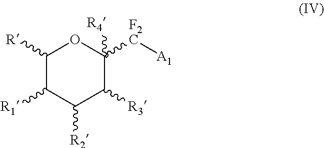

in which R′, R₁′, R₂′, R₃′ and R₄′ are as defined above and A₁ represents CHO or C(OA₂)(OA₃) with A₂ and A₃ representing, independently of one another, H, (C₁-C₆)alkyl or aryl-(C₁-C₆)alkyl; notably with A₂=H and A₃ representing (C₁-C₆)alkyl or aryl-(C₁-C₆)alky, notably (C₁-C₆)alkyl, with a compound of the following formula (V):

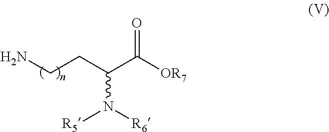

or a salt thereof, such as a hydrochloride, in which n, R₅′, R₆′ and R₇ are as defined above.

This reaction can be carried out in toluene at the reflux temperature in the presence of a Dean-Stark apparatus.

This reaction can also be carried out in the presence of a base, such as triethylamine, or NaHCO₃ and optionally a dessicant agent, such as MgSO₄. In this case dichloromethane or dichloroethane can be used as solvent. The base can be also PsNEt₂ (diethylaminomethyl-polystyrene) to facilitate the purification. In this case, the solvent can be dichloroethane.

The reaction between compounds of formulas (IV) and (V) and the reduction of compounds (III) can be one-pot.

In the case of these reactions, advantageously R₅′≠H and/or R₆′≠H, R′≠CH₂OH, R₁′≠OH, R₂′≠OH, R₃′≠OH, and R₄′≠OH. Thus, to prepare compounds which such substituents, the OH or NH₂ functions should be preferably protected by a protecting group as defined above before performing the reaction between the compounds of formulas (IV) and (V). Of course, the NH₂ group of the CH₂—(CH₂)ₙ—NH₂ moiety remains unprotected (it can be in the form of a salt) in order to be able to react with A₁.

The compound of formula (IV) can be prepared as disclosed in WO2015/140178.

The compound of formula (V) can be prepared according to methods disclosed in the examples below.

The compound of formula (II) can be prepared also by reacting a compound of the following formula (VI):

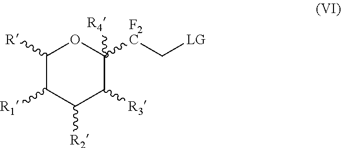

in which R′, R₁′, R₂′, R₃′ and R₄′ are as defined above and LG represents a leaving group, notably a sulfonate such as a triflate,
with a compound of formula (V) as defined above or a salt thereof.

The substitution reaction is advantageously carried out in the presence of a base such as K₂CO₃. The reaction can be carried out in a solvent such as DMF.

In the case of this reaction, advantageously R₅′≠H and/or R₆′≠H, R′≠CH₂OH, R₁′≠OH, R₂′≠OH, R₃′≠OH, and R₄′≠OH. Thus, to prepare compounds which such substituents, the OH or NH₂ functions should be preferably protected by a protecting group as defined above before performing the reaction between the compounds of formulas (VI) and (V).

The compound of formula (VI) can be prepared as disclosed in WO2015/140178.

The compound of formula (II) can be prepared also by reacting a compound of the following formula (VII):

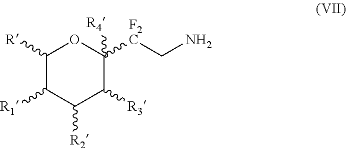

in which R′, R₁′, R₂′, R₃′ and R₄′ are as defined above, with a compound of the following formula (VIII):

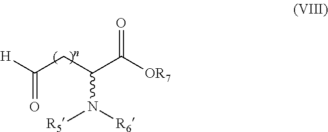

in which n, R₅′, R₆′ and R₇ are as defined above.

The reduction reaction can be carried out in the presence of a borohydride such as NaBH₃CN or NaBH(OAc)₃.

The reaction can be carried out in a solvent such as dichloroethane.

In the case of this reaction, advantageously R₅′≠H and/or R₆′≠H, R′≠CH₂OH, R₁′≠OH, R₂′≠OH, R₃′≠OH, and R₄′≠OH. Thus, to prepare compounds which such substituents, the OH or NH₂ functions should be preferably protected by a protecting group as defined above before performing the reaction between the compounds of formulas (VII) and (VIII).

The compound of formula (VII) can be prepared according to methods disclosed in the examples below. The compound of formula (VIII) is commercially available or easily prepared by the skilled person (as described in Journal of Organic Chemistry 1998, 63, 3741-3744).

Step (b):

The protected forms will comprise protected group(s), in particular OH group(s) protected with any O-protecting group such as defined previously, in particular a benzyl group, and/or NH₂ group(s) protected with one or two N-protecting group(s) such as defined previously, in particular a Cbz or Boc group.

The conditions of deprotection are well-known to the one skilled in the art (e.g. “Greene's Protective Groups In Organic Synthesis”, 4^{th} edition, 2007, John Wiley & Sons, Hoboken, New Jersey). For example, the deprotection of an OH group protected with a benzyl group or of a NH₂ group protected with a Cbz group can be performed in the presence of H₂ and a catalyst such as Pd/C.

The deprotection step can be carried out after and/or during step (a).

The deprotection step can be carried out after, before and/or during step (c).

Step (c):

The salification or solvation step can be carried out by methods well known to the one skilled in the art, in particular by reaction of the compound of formula (I) obtained in step (a) or (b) with an organic or inorganic acid, an organic or inorganic base or a solvent, as defined previously.

The solvent can be notably the solvent used in the last step of the preparation of the compound according to the invention, in particular the solvent used in step (a) or (b).

Thus, steps (a) and/or (b) and (c) can be carried out in a single step, without isolating intermediate compounds.

The compound obtained by the process according to the invention can be separated from the reaction medium by methods well known to the person skilled in the art, such as by extraction, evaporation of the solvent or by precipitation or crystallisation (followed by filtration).

The compound can be also purified if necessary by methods well known to the person skilled in the art, such as by recrystallization, by distillation, by ion exchange purification (DOWEX® 50Wx8), by chromatography on a column of silica gel or by high performance liquid chromatography (HPLC).

Cosmetic or Pharmaceutical Compositions

The present invention relates also to a cosmetic or pharmaceutical (e.g. dermatological) composition comprising at least one compound of formula (I) as defined above and at least one physiologically acceptable excipient.

Such a composition is more particularly intended for a topical (e.g. transdermal) administration or a parenteral (e.g. subcutaneous or intradermal) administration, preferably a topical administration, in particular on the skin, including the scalp skin, or an injection, in particular a subcutaneous or intradermal injection.

Such a composition can thus be a solution, a dispersion, an emulsion, an oil, an ointment, a shampoo, a paste, a cream, a lotion, a milk, a foam, a gel, a suspension, a spray, a serum, a patch, a stick or a mask.

The composition of the invention may comprise one or several additive(s) as excipient(s), such as suspending agents, wetting agents, antioxidants, emollients, other moisturizing agents, thickening agents, chelating agents, buffering agents, tonicity adjusting agents, fragrances, preservatives, pigments or colorants, opacifiers or mattifying agents. Such additives are conventional to those of skill in the art and exemplified below.

Suspending agents can be for example an alginate, sodium carboxymethyl cellulose, methyl cellulose, hydroxyl methyl cellulose, hydroxyl ethyl cellulose, hydroxylpropyl methyl cellulose, microcrystalline cellulose, a gum such as acacia, tragacanth or xanthan gum, gelatin, a carrageenan, polyvinyl pyrrolidone.

Wetting agents can be glycerin, propylene glycol or also nonionic surfactants such as a lecithin, a polysorbate or a poloxamer.

Antioxidants can be used to protect ingredients of the composition from oxidizing agents that are included within or come in contact with the composition. Examples of antioxidants include ascorbic acid, ascorbyl palmitate, citric acid, acetylcysteine, sulfurous acid salts (bisulfite, metabisulfite), sodium formaldehyde sulfoxylate, monothioglycerol, thiourea, butylated hydroxyanisole, butylated hydroxytoluene, potassium propyl gallate, octyl gallate, dodecyl gallate, phenyl-α-naphthyl-amine, and tocopherols such as α-tocopherol.

Emollients are agents that soften and smooth the skin. Examples of emollients include oils and waxes such as siloxanes such as dimethicone and derivatives thereof, microcrystalline wax, polyethylene, triglyceride esters such as those of castor oil, cocoa butter, safflower oil, corn oil, olive oil, cod liver oil, almond oil, palm oil, squalene, and soybean oil, acetylated monoglycerides, ethoxylated glycerides, fatty acids, alkyl esters of fatty acids, alkenyl esters of fatty acids, fatty alcohols, fatty alcohol ethers, ether-esters, lanolin and derivatives of lanolin, polyhydric alcohol esters, wax esters such as beeswax, vegetable waxes, phospholipids, sterols, isopropyl palmitate or glyceryl stearate.

A moisturising agent increases the moisture content of the skin and keeps it soft and smooth. It can be for example urea, an amino acid, lactic acid and its salts (such as sodium lactate), glycerol (also called glycerin), propylene glycol, butylene glycol, PEG (polyethylene glycol—such as PEG-4 to PEG-32), sorbitol, xylitol, maltitol, mannitol, polydextrose, collagen, elastin, hyaluronic acid and its salts (such as sodium or potassium salts), pectin, gelatin, chitosan, aloe vera, honey, etc.

Thickening agents are used to increase the viscosity and thickness of the composition. Examples of thickening agents include lipid thickening agents such as Cetyl Alcohol, Stearyl Alcohol, Myristyl Alcohol, Carnauba Wax, or Stearic acid; naturally derived thickening agents such as Cellulose derivatives like Hydroxyethylcellulose, Guar gum, Locust Bean Gum, Xanthan Gum, or Gelatin; mineral thickening agents such as Silica, Bentonite, or Magnesium Aluminum Silicate; synthetic thickening agents such as Carbomer; ionic thickening agents such as NaCl.

Chelating agents can be an ethylene diamine tetraacetic acid (EDTA) salt.

Buffering agents can be acetate, citrate, tartrate, phosphate, triethanolamine (TRIS).

Examples of fragrances or perfume include peppermint, rose oil, rose water, aloe vera, clove oil, menthol, camphor, eucalyptus oil, and other plant extracts. To eliminate certain odors from compositions, masking agents may be used.

Preservatives can be used to protect the composition from degradation. Examples of preservatives include phenol, cresol, chlorobutanol, phenoxyethanol, butylparaben, propylparaben, ethylparaben, methylparaben, propyl paraben, benzalkonium chloride, benzethonium chloride, benzoic acid, benzyl alcohol, and mixtures thereof such as liquipar oil. However, the composition of the present invention can be preservative free.

Pigments or colorants are used to modify the color of the composition, such as to obtain a white composition.

Opacifiers, such as titanium oxide, are used in clear or transparent composition in order to render it opaque. The present invention can thus be clear or opaque according to the use or not of an opacifier.

Mattifying agents are ingredients that make the skin matt, which prevent it from shining. It can be for example talc, silica, rice powder, or a mixture thereof, notably in a micronized form.

The one skilled in the art will be able to adapt the amount of the compound of formula (I) according to the invention in the cosmetic or pharmaceutical (e.g. dermatological) composition in order to obtain the desired effect.

For parenteral, in particular subcutaneous or intradermal, administration, the cosmetic or pharmaceutical composition according to the invention can be more particularly in the form of an aqueous suspension or solution which is advantageously sterile. Such parenteral (e.g. subcutaneous) compositions will contain advantageously a physiologically acceptable medium, generally based on an isotonic saline solution, i.e. 0.9% NaCl aqueous solution (normal saline). Non-aqueous water miscible co-solvent, such as ethanol, glycerin, propylene glycol or n-lactamide, can also be used. The parenteral composition of the invention can also comprise one or more additive(s), such as suspending agents, wetting agents, preservatives, antioxidants, chelating agents, buffering agents, tonicity adjusting agents, etc. Such additives are conventional to those of skill in the art and examples are mentioned above.

For topical administration, the cosmetic or pharmaceutical composition according to the invention can be in the usual forms for a topical administration including creams, lotions, serums, gels, foams, dispersions, suspensions, emulsions, sprays, shampoos, masks, milks, etc. The active ingredient can be administered in unit forms for administration, mixed with conventional pharmaceutical carriers, to animals, preferably mammals including humans. Such topical compositions generally contain a physiologically acceptable medium, notably based on water or a solvent such as alcohols (for ex. ethanol), ethers or glycols. The topical composition of the invention can also comprise one or more additive(s), such as antioxidants, emollients, other moisturizing agents, thickening agents, fragrances, preservatives, pigments or colorants, or opacifiers.

Such additives are conventional to those of skill in the art and examples are mentioned above.

The cosmetic or pharmaceutical (e.g. dermatological) composition is intended in particular:
for the treatment and/or prevention of skin aging, skin protection, or skin regeneration;
for skin plumping and/or skin volumizing and/or skin densifying and/or wrinkle filling and/or skin or hair moisturizing and/or skin or hair relipiding and/or stimulation of hair growth;
for the treatment of dry skin and/or atopic dermatitis and/or atopic eczema and/or psoriasis; or
for the treatment and/or prevention of a fibrosis disease (e.g. an excessive scar such as a keloid or hypertrophic scar) or for healing;
for the treatment of inflammation (e.g. chronic, low-grade inflammation, notably that develops in various aging tissues and referred as “inflammaging”).

Cosmetic or pharmaceutical applications According to a first aspect, the present invention relates to a compound of formula (I) or a cosmetic or pharmaceutical (e.g. dermatological) composition according to the invention for use in the treatment and/or prevention of skin aging, skin protection, or skin regeneration.

The present invention relates also to a use, such as a cosmetic use, of a compound of formula (I) or a cosmetic or pharmaceutical (e.g. dermatological) composition according to the invention for the treatment and/or prevention of skin aging, skin protection, or skin regeneration.

The present invention relates also to a method, such as a cosmetic method, for the treatment and/or prevention of skin aging, skin protection, or skin regeneration, by applying a compound of formula (I) or a cosmetic or pharmaceutical (e.g. dermatological) composition according to the invention to the skin.

The present invention relates also to a method for the treatment and/or prevention of skin aging, skin protection, or skin regeneration, by applying to the skin of a person in need thereof of an affective amount of a compound of formula (I) or a cosmetic or pharmaceutical (e.g. dermatological) composition according to the invention.

Indeed, it has been demonstrated that the compounds of formula (I) according to the invention have properties of increasing the growth (proliferation) of skin cell in particular under stress conditions, protecting them from different stresses and especially oxidative stress, reducing inflammation, through the inhibition of cytokine release such as IL6, promoting extracellular matrix remodelling, inducing hyaluronic acid synthesis and promoting lipogenesis.

In such use or method, the compound of formula (I) or cosmetic or pharmaceutical (e.g. dermatological) composition according to the invention can be applied topically on the skin.

According to a second aspect, the present invention relates to the use of a compound of formula (I) or a cosmetic or pharmaceutical (e.g. dermatological) composition according to the invention for skin plumping and/or skin volumizing and/or skin densifying and/or wrinkle filling and/or skin or hair moisturizing and/or skin or hair relipiding and/or stimulation of hair growth.

The invention relates also to a method for skin plumping and/or skin volumizing and/or skin densifying and/or wrinkle filling and/or skin or hair moisturizing and/or skin or hair relipiding and/or stimulation of hair growth comprising the administration, notably topically onto the skin (including the scalp skin for the stimulation of hair growth) or subcutaneously or intradermally, of an effective amount of a compound of formula (I) or a cosmetic or pharmaceutical (e.g. dermatological) composition according to the invention.

The invention relates also to a compound of formula (I) or a cosmetic or pharmaceutical (e.g. dermatological) composition according to the invention for use for skin plumping and/or skin volumizing and/or skin densifying and/or wrinkle filling and/or skin or hair moisturizing and/or skin or hair relipiding and/or stimulation of hair growth.

The invention relates also to the use of a compound of formula (I) or a cosmetic or pharmaceutical (e.g. dermatological) composition according to the invention, for the manufacture of a cosmetic or dermatological composition intended for skin plumping and/or skin volumizing and/or skin densifying and/or wrinkle filling and/or skin or hair moisturizing and/or skin or hair relipiding and/or stimulation of hair growth.

Indeed, it has been demonstrated that the compounds of formula (I) according to the invention have an activity of increasing the volume of adipose tissue notably through the proliferation of preadipocytes, through the synthesis of lipids such as cholesterol, through the reduction of inflammation, with the inhibition of cytokine release such as IL6, through the synthesis of hyaluronic acid, and an activity of hair growth in particular through the synthesis of lipids and through the proliferation of fibroblast.

In such use or method, the compound of formula (I) or cosmetic or pharmaceutical (e.g. dermatological) composition according to the invention can be applied on the skin, including the scalp, topically, subcutaneously or intradermally, preferably subcutaneously or intradermally.

According to a third aspect, the present invention relates to a compound of formula (I) or a cosmetic or pharmaceutical (e.g. dermatological) composition according to the invention for use in the treatment of dry skin and/or atopic dermatitis and/or atopic eczema and/or psoriasis.

The invention relates also to the use of a compound of formula (I) or a cosmetic or pharmaceutical (e.g. dermatological) composition according to the invention for the treatment of dry skin and/or atopic dermatitis and/or atopic eczema and/or psoriasis.

The invention relates also to the use of a compound of formula (I) or a cosmetic or pharmaceutical (e.g. dermatological) composition according to the invention for the manufacture of a cosmetic or pharmaceutical (e.g. dermatological) composition intended for the treatment of dry skin and/or atopic dermatitis and/or atopic eczema and/or psoriasis.

The invention relates also to a method for the treatment of dry skin and/or atopic dermatitis and/or atopic eczema and/or psoriasis comprising the administration to a person in need thereof of an effective amount of a compound of formula (I) or a cosmetic or pharmaceutical (e.g. dermatological) composition according to the invention.

Indeed, as reported in the literature (J. Invest. Dermatol. 1991, 96, 523-526; Contact Dermatitis 2008, 58, 255-262; Skin Pharmacol. Physiol. 2015, 28, 42-55), such pathologies are associated with a decrease of lipid synthesis leading to a skin barrier impairment. It has been demonstrated that the compounds of formula (I) according to the invention are useful in lipid synthesis so that such compounds can be used in the treatment of these pathologies by stimulating the lipid synthesis notably by keratinocytes.

The administration of the compound of formula (I) or cosmetic or pharmaceutical (e.g. dermatological) composition according to the invention is advantageously topical or parenteral (e.g. subcutaneous or intradermal), preferably topical, in the case of the treatment of dry skin and/or atopic dermatitis and/or atopic eczema and/or psoriasis.

According to a fourth aspect, the present invention relates also to a compound of formula (I) or a cosmetic or pharmaceutical (e.g. dermatological) composition according to the invention for use in the treatment and/or prevention of a fibrosis disease, in particular an excessive scar such as a keloid or hypertrophic scar, or for use in healing.

The present invention relates also to the use of a compound of formula (I) or a cosmetic or pharmaceutical (e.g. dermatological) composition according to the invention for the manufacture of a cosmetic or pharmaceutical (e.g. dermatological) composition intended for the treatment and/or prevention and of a fibrosis disease, in particular an excessive scar such as a keloid or hypertrophic scar, or for healing.

The present invention relates also to the use of a compound of formula (I) or a cosmetic or pharmaceutical (e.g. dermatological) composition according to the invention in the treatment and/or prevention of a fibrosis disease, in particular an excessive scar such as a keloid or hypertrophic scar, or for healing.

The present invention relates also to a method of treating and/or preventing a fibrosis disease, in particular an excessive scar such as a keloid or hypertrophic scar, or for healing, comprising the administration to a person in need thereof of an effective amount of a compound of formula (I) or a cosmetic or pharmaceutical (e.g. dermatological) composition according to the invention.

Indeed, it has been demonstrated that the compounds of formula (I) according to the invention have a role in the regulation of several genes involved in the mechanism of healing and treating/preventing fibrosis diseases such as keloids (e.g. genes involved in extracellular matrix organization or fibrogenesis inhibition).

The compound of formula (I) or cosmetic or pharmaceutical (e.g. dermatological) composition according to the invention can be used in combination with, and more particular after, a laser or surgical treatment. Indeed, a patient suffering from a fibrosis disease, in particular an excessive scar such as a keloid or hypertrophic scar, can be first treated with laser or by surgery to eliminate the excess fibrous connective tissue and then a compound of formula (I) or a cosmetic or pharmaceutical (e.g. dermatological) composition according to the invention can be applied topically on the wound during its healing in order to prevent the reappearance of the excess fibrous connective tissue.

The administration of the compound of formula (I) or cosmetic or pharmaceutical (e.g. dermatological) composition according to the invention is advantageously topical or parenteral (e.g. subcutaneously or intradermally), preferably topical, in the case of the treatment and/or prevention of a fibrosis disease or of healing.

According to a fifth aspect, the present invention relates also to a compound of formula (I) or a cosmetic or pharmaceutical (e.g. dermatological) composition according to the invention for use in the treatment of inflammation and especially chronic, low-grade inflammation, notably that develops in various aging tissues and referred as “inflammaging”.

The present invention relates also to the use of a compound of formula (I) or a cosmetic or pharmaceutical (e.g. dermatological) composition according to the invention for the manufacture of a cosmetic or pharmaceutical (e.g. dermatological) composition intended for the treatment of inflammation and especially chronic, low-grade inflammation, notably that develops in various aging tissues and referred as “inflammaging”.

The present invention relates also to the use of a compound of formula (I) or a cosmetic or pharmaceutical (e.g. dermatological) composition according to the invention in the treatment of inflammation and especially chronic, low-grade inflammation, notably that develops in various aging tissues and referred as “inflammaging”.

The present invention relates also to a method of treating inflammation and especially chronic, low-grade inflammation, notably that develops in various aging tissues and referred as “inflammaging”, comprising the administration to a person in need thereof of an effective amount of a compound of formula (I) or a cosmetic or pharmaceutical (e.g. dermatological) composition according to the invention.

Indeed, it has been demonstrated that the compounds of formula (I) according to the invention have a role in the regulation of several genes involved in the mechanism of inflammation (e.g. genes involved in inflammatory response and chronic inflammatory disorder inhibition) and in the reduction of inflammation through the inhibition of IL6 release in tissues (e.g. adipocytes).

The compound according to the invention can thus be useful also to treat obesity or, in a patient suffering from obesity, to increase weight loss, or more particularly fat loss, and to prevent the onset of a metabolic syndrome such as type 2 diabetes.

The present invention relates thus also to a compound of formula (I) or a cosmetic or pharmaceutical (e.g. dermatological) composition according to the invention for use in the treatment of obesity or for use, in a patient suffering from obesity, in a method of increasing weight loss, or more particularly fat loss, or in the prevention of the onset of a metabolic syndrome such as type 2 diabetes.

The present invention relates also to the use of a compound of formula (I) or a cosmetic or pharmaceutical (e.g. dermatological) composition according to the invention for the manufacture of a cosmetic or pharmaceutical (e.g. dermatological) composition intended for the treatment of obesity or, in a patient suffering from obesity, for increasing weight loss, or more particularly fat loss, or for preventing the onset of a metabolic syndrome such as type 2 diabetes.

The present invention relates also to the use of a compound of formula (I) or a cosmetic or pharmaceutical (e.g. dermatological) composition according to the invention in the treatment of obesity or, in a patient suffering from obesity, for increasing weight loss, or more particularly fat loss, or for preventing the onset of a metabolic syndrome such as type 2 diabetes.

The present invention relates also to a method of treating obesity or, in a patient suffering from obesity, of increasing weight loss, or more particularly fat loss, or of preventing the onset of a metabolic syndrome such as type 2 diabetes, comprising the administration to a person in need thereof of an effective amount of a compound of formula (I) or a cosmetic or pharmaceutical (e.g. dermatological) composition according to the invention.

Dressing

The present invention concerns also a dressing comprising a pad, compress or sponge impregnated with a cosmetic or pharmaceutical (e.g. dermatological) composition according to the present invention as defined above.

Such a dressing can be applied to an injury/a wound during the healing step in order to prevent or reduce the appearance of keloids or hypertrophic scars. Thus, it can be for use in the treatment and/or prevention, notably in the prevention, of a fibrosis disease, in particular an excessive scar such as a keloid or hypertrophic scar, or for use in healing.

It is thus preferably sterile.

Such a dressing can be more particularly a pressure dressing.

The pad, compress or sponge can be made of various materials, preferably absorbent materials, such as cotton, gauze, a porous polymer material, or a combination thereof, notably cotton and/or gauze.

It can also comprise a bandage or adhesive means in order to maintain the pad or compress in close contact with the injury or wound.

This dressing can be used in combination with, and more particular after, a laser or surgical treatment. Indeed, a patient suffering from a fibrosis disease, in particular an excessive scar such as a keloid or hypertrophic scar, can be first treated with laser or by surgery to eliminate the excess fibrous connective tissue and then a dressing according to the invention can be applied on the wound during its healing in order to prevent the reappearance of the excess fibrous connective tissue.

Preservation, Protection, Regeneration of a Biological Material or a Microorganism

The present invention relates also to the use of a compound of formula (I) as defined above for the preservation and/or protection and/or regeneration of a biological material or a microorganism.

The present invention relates also to a method of preservation and/or protection of a biological material or a microorganism by placing said biological material or microorganism in a medium containing a compound of formula (I) as defined above.

Indeed, it has been demonstrate that the compounds of formula (I) according to the invention have properties to promote cells growth and to protect cells from stress and especially oxidative stress.

In particular, a biological material or a microorganism can be protected/preserved when placed at a temperature below 37° C., such as below 0° C., notably in conditions of cryopreservation in particular for biological materials such as human organs, tissues (e.g. for transplant), body fluids or cells.

The cryopreservation of a biological material or a microorganism implies to cool to sub-zero temperatures the biological material or microorganism, and notably at a temperature of about −196° C. by using liquid nitrogen.

The biological material can be in particular cells, a tissue, a body fluid or an organ. For example, the biological material can be an organ or a tissue (e.g. skin or, in the case of hair graft, a follicular unit, i.e. a scalp part comprising 1 to 4 hair follicles) intended to be grafted.

The microorganism can be in particular a prokaryotic or eukaryotic microorganism, being notably unicellular or pluricellular.

The microorganism can be notably chosen among bacteria, fungi, including yeasts, algae, viruses, including phages, microparasites (also called parasitic microorganisms) and protozoa.

Culture, Storage and/or Preservation Medium

The present invention relates also to a culture, storage and/or preservation medium comprising at least one compound of formula (I) as defined above.

The culture, storage and/or preservation medium can be liquid or in the form of a gel. It contains thus water. However, the medium can be in a dehydrated form which can be rehydrated by water addition.

It can contain one or several components of the group consisting of co-solvents (e.g. dimethylsulfoxyde (DMSO)), salts (for ex. NaCl, MgCl₂, ZnCl₂, MnCl₂, CuCl₂, K₂PO₄, KH₂PO₄, K₂HPO₄, Na₂S₂O₃, K₂SO₄, MgSO₄, KNO₃, Ca(NO₃)₂, Na₂CO₃, NaHCO₃, etc.), carbon sources such as carbohydrates (for ex. glucose, lactose or sucrose) or polyols (for ex mannitol or glycerol), vitamins (for ex. vitamins B1, B2, B6, B12, B3, B5, B9, B7, C, A, D, E and K), nitrogen and amino acid sources (for ex. peptones, beef or yeast extract, serum, etc.), growth factors (for ex. insulin, transferrin, fibonectin, albumin), differentiating factors, antibiotics and antimycotics (also called antibacterial and antifungal agents—e.g. actinomycin D, amphotericin B, ampicillin, carbenicillin, cefotaxime, fosmidomycin, gentamicin, kanamycin, neomycin, streptomycin, penicillin, polymixin B), hormones, cytokines and trace elements.

Other additives can be present such as indicators (of pH for example), inhibitors, etc.

When it is in the form of a gel, the culture medium can further comprise a gelling agent such as agar, gelatine, silica gel, etc.

The present invention relates also to the use of a compound of formula (I) as defined above as an adjuvant in a culture, storage and/or preservation medium.

The culture, storage and/or preservation medium is intended for the culture, storage and/or preservation of a biological material or of a microorganism. The biological material will be more particularly cells or tissues in the case of a culture medium.

The present invention is illustrated by the following non-limitative examples.

### EXAMPLES

The following abbreviations have been used:
Ac: Acetyl (COCH₃)
BHA: Butylated hydroxyanisole
Bn: Benzyl (CH₂Ph)
Boc: tert-Butyloxycarbonyl
Cbz: Benzyloxycarbonyl (CO₂CH₂Ph)
cpm: Counts per minute
DAPI: 4′,6-Diamidino-2-Phenylindole, Dihydrochloride
DCE: Dichloroethane
DCM: Dichloromethane
DMEM: Dulbecco's Modified Eagle Medium
DMF: Dimethylformamide
DIPEA: N,N-Diisopropylethylamine
EBSS: Earle's Balanced Salt Solution
EDTA: Ethylenediaminetetraacetic acid
ESI: Electrospray ionisation
FBS: Fetal Bovine Serum
FCS: Fetal Calf Serum
HATU: 1-[Bis(dimethylamino)methylene]-1H-1,2,3-triazolo[4,5-b]pyridinium 3-oxid hexafluorophosphate
H-PTFE: Hydrophilized polytetrafluoroethylene
LDH: Lactate Deshydrogenase
Me: Methyl
NHDF: Normal human dermal fibroblasts
NHEK: Normal human epidermal keratinocytes
NMR: Nuclear Magnetic Resonance
OD: Optical density
PBS: Phosphate buffered saline
PsNEt₂: Diethylaminomethyl-polystyrene
RMA: Robust Multiarray Analysis
RNA: Ribonucleic acid
ROS: Reactive oxygen species
RPMI medium: Roswell Park Memorial Institute medium
Tf: Trifluoromethanesulfonyl (SO₂CF₃)
THE: Tetrahydrofuran

### 1. Synthesis of the Compounds According to the Invention

It should be noted that the compounds according to the invention where R₄═R₁═OH can be obtained in the form of a mixture of tautomer forms as explained in the description above. For practical reasons, these compounds are represented by their pyranose form.

### 1.1. Synthesis of Compound 6 According to a First Synthesis Route

Compound 6 can be prepared according to the following synthesis route:

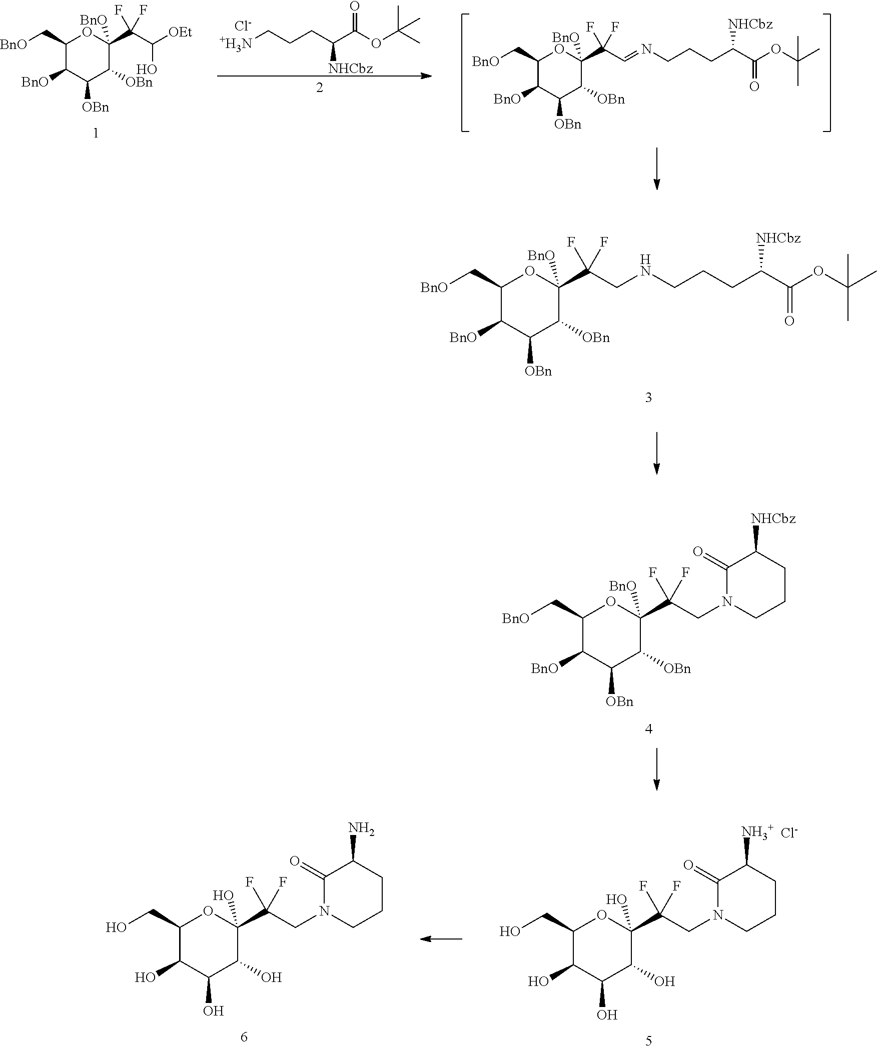

### Synthesis of Intermediate Compound 1:

The preparation of compound 1 is disclosed in WO2015/140178 (cf. compound 2).

### Synthesis of Intermediate Compound 2:

Compound 2 is prepared according to the following two steps:

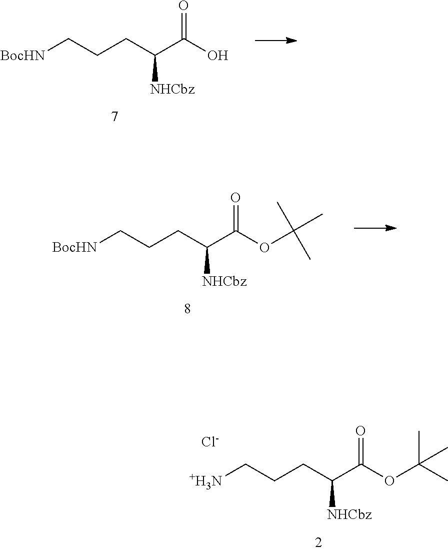 [SMILES: CC(C)(C)OC(=O)NCCC[C@H](NC(=O)OCc1ccccc1)C(=O)O ; CC(C)(C)OC(=O)NCCC[C@H](NC(=O)OCc1ccccc1)C(=O)OC(C)(C)C ; [Cl-] ; CC(C)(C)OC(=O)[C@H](CCC[NH3+])NC(=O)OCc1ccccc1]

Compound 8 is prepared from commercially available compound 7 according to the method disclosed in Organic Letters 2006, 8, 17, 3865-3868.

Compound 2 is then obtained from compound 8 according to a protocol disclosed in J. Org. Chem. 1994, Vol. 59, No. 11, 3216-3218 as follows.

Compound 8 (1 eq., 1.0 g, 2.37 mmol) was dissolved in a solution of HCl (1M in AcOEt, 2.0 eq., 4.73 mL, 4.73 mmol). The reaction mixture was stirred at room temperature for 18 h. HCl (1M in AcOEt, 1 eq., 2.37 mL, 2.37 mmol) was added again to complete the reaction. The reaction mixture was stirred for an additional 5 h. The mixture was then concentrated and co-evaporated with Et₂O to give 2.37 g of compound 2 (67% purity). The material was engaged in the next step without purification.

¹H NMR (MeOD, 300 MHz): 1.44 (s, 9H); 1.54-2.10 (m, 4H); 2.93 (m, 2H); 4.10 (m, 1H); 5.10 (s, 2H); 7.29-7.38 (m, 5H).

Mass (ESI+): 323.2 [M+H]⁺ (NH₂ form)

### Synthesis of Intermediate Compound 3:

To a solution of compound 1 (1 eq., 1.20 g, 1.59 mmol) in DCE (12.6 mL) under inert atmosphere were sequentially added PsNEt₂ (Diethylaminomethyl-polystyrene 3.2 mmol/g, 2.0 eq., 1.10 g, 3.18 mmol), compound 2 (67% purity, 1.0 eq., 0.85 g, 1.59 mmol) and MgSO₄ (5 eq., 0.96 g, 7.95 mmol). The reaction was then refluxed for 16 h. The mixture was cooled to room temperature and then rapidly filtered and rinsed with 10 mL of DCE. The obtained yellow solution was transferred in a round bottom flask and was cooled to 0° C. under inert atmosphere. To this solution were added by portions sodium triacetoxyborohydride (2.0 eq., 0.67 g, 3.17 mmol) and acetic acid (1.0 eq., 0.09 mL, 1.59 mmol). The reaction was stirred for 30 minutes at 0° C. and was then allowed to warm up to room temperature and was stirred for 3 hours.

Aqueous saturated solution of NaHCO₃ was added and the mixture was vigorously stirred for 5 minutes. The mixture was then extracted with DCM (3×). The combined organic layers were dried over Na₂SO₄, filtered and concentrated.

The resulting crude oil was purified by chromatography (SiO₂ cartridge, cyclohexane/AcOEt: 90/10 to 80/20 to give compound 3 (1.05 g, 95% purity).

¹⁹Fdec NMR (CDCl₃, 282.5 MHz): −109.5 (d, 258 Hz, 1F, CF₂); −110.4 (d, 258 Hz, 1F, CF₂).

Mass (ESI+): 1015.5 [M+H]⁺; 1037.5 [M+Na]⁺

### Synthesis of Intermediate Compound 4:

In a sealed tube, a solution of compound 3 (1 eq., 95% purity, 1.05 g, 0.98 mmol) in toluene (11.4 mL) and acetic acid (10.5 eq., 0.59 mL, 10.34 mmol) was heated at reflux for 18 h. The reaction mixture was concentrated. The residue was purified by flash chromatography (80 g SiO₂ cartridge, cyclohexane/EtOAc 90/10 to 55/45) to afford compound 4 (0.83 g, 85% purity, 55% over 3 steps) as colorless gum.

¹⁹F NMR (CDCl₃, 282.5 MHz): −108.0 (br dd, 256 Hz, 33 Hz, 1F); −112.3 (br dd, 256 Hz, 26 Hz, 1F).

¹⁹F dec NMR (CDCl₃, 282.5 MHz): −108.0 (d, 256 Hz, 1F); −112.3 (d, 256 Hz, 1F).

Mass (ESI+): 958.5 [M+NH4]+; 963.5 [M+Na]+; 979.5 [M+K]+

### Synthesis of Intermediate Compound 5:

Palladium (loading 10 wt %, support activated carbon, 0.10 eq., 0.11 g, 0.10 mmol) was added to a solution of compound 4 (1 eq., 0.93 g, 0.99 mmol) in THE (38 mL), previously degassed with nitrogen. A solution of HCl (2M in water, 4.0 eq., 2.0 mL, 3.95 mmol) was then added. The mixture was placed under hydrogen atmosphere and was stirred for 18 h. The reaction was degassed with nitrogen prior to be filtered (0.45 μm, H-PTFE) to remove the palladium residues. The filter was washed with a mixture of THE and water and the combined solution was concentrated to remove the THF. The residue was then diluted with water and the solution was filtered (0.2 μm, H-PTFE) before being freeze dried to afford compound 5 (0.45 g) as a white powder. The material was engaged in the next step without purification.

Compound 5 is obtained as a mixture of the two following tautomer forms named Form 1 and Form 2:

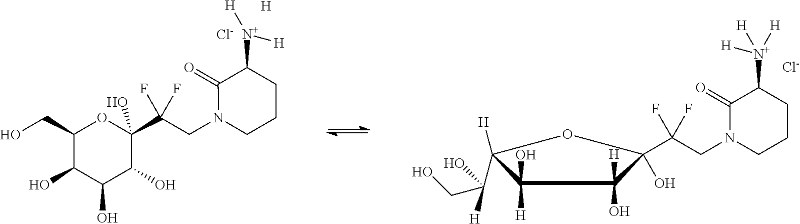

¹⁹F dec NMR (D₂O, 282.5 MHz):

Form 1 (55%): −115.7 (ddd, 255 Hz, 25 Hz, 8 Hz, 1F, CF₂; −118.5 (ddd, 251 Hz, 24 Hz, 9 Hz, 1F, CF₂)

Form 2 (45%): −115.0 (ddd, 251 Hz, 27 Hz, 8 Hz, 1F, CF₂); −116.5 (ddd, 255 Hz, 26 Hz, 7 Hz, 1F, CF₂)

Mass (ESI⁻): 391.0 (M−H)⁻

### Synthesis of Compound 6:

Amberlite® IRA-67 (previously washed with water, 1.73 g) was added to a solution of compound 5 (0.45 g, 1.15 mmol) in water (30 mL). The solution was stirred for 1 h30 at room temperature. The pH of the solution was measured (pH=6.8-7.0) and the mixture was filtered (0.2 μm, H-PTFE). The filtrate was then freeze-dried to afford compound 6 (0.28 g, 69% yield) as an off-white powder.

Compound 6 is obtained as a mixture of the two following tautomer forms named Form 1 and Form 2:

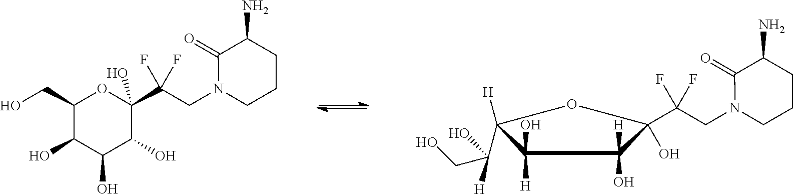

¹⁹F NMR (D₂O, 282.5 MHz):

Form 1 (57%): −118.2 (ddd, 252 Hz, 23 Hz, 11 Hz); −115.5 (ddd, 252 Hz, 24 Hz, 10 Hz).

Form 2 (43%): −116.4 (ddd, 253 Hz, 27 Hz, 15 Hz, 1F, CF₂); −115.2 (ddd, 253 Hz, 27 Hz, 15 Hz, 1F, CF₂)

Mass (ESI+): 357.1 [M+H]⁺

### 1.2. Synthesis of Compound 6 According to a Second Synthesis Route

Compound 6 can be prepared according to the following synthesis route:

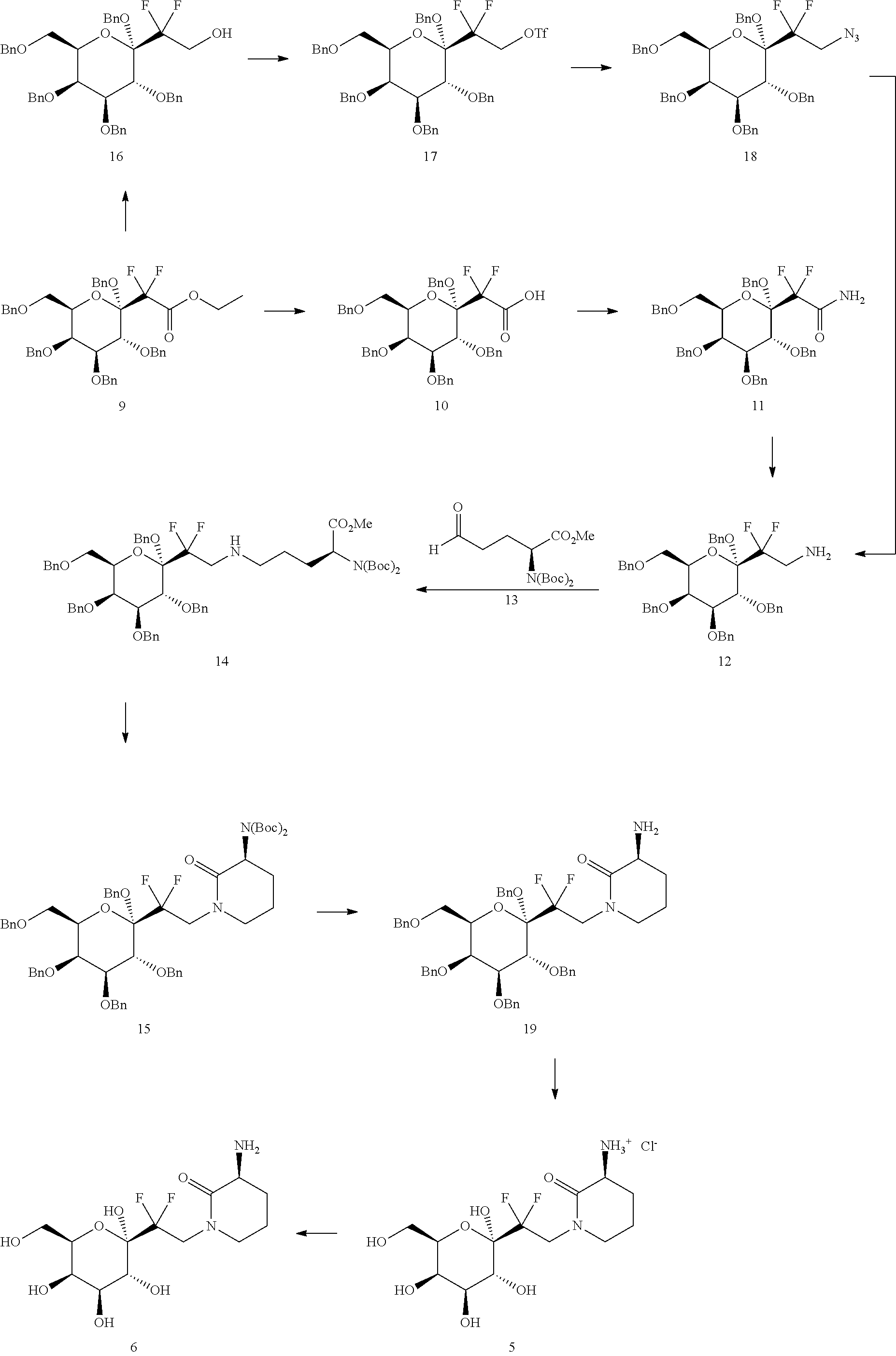

### Synthesis of Intermediate Compound 10:

LiOH (4.5 eq., 1.29 g, 0.90 mmol) was added to a solution of compound 9 (1 eq., 10.0 g, 12 mmol—compound prepared according to the process disclosed in WO 2012/085221 (see synthesis of compound 15)) in a mixture of THE (98 mL) and water (21.5 mL). The reaction mixture was stirred at room temperature for 18 h. Brine was added and 1M HCl until acidic pH was reached. The aqueous layer was then extracted with AcOEt and the combined organic layers were dried over Na₂SO₄, filtered and concentrated to afford crude compound 10 (10.9 g, 126% yield, 80% purity) as a yellow oil. The material was engaged in the next step without purification.

¹⁹F NMR (CDCl₃, 282.5 MHz): −109.3 (d, 269 Hz, 1F, CF₂); −111.56 (d, 269 Hz, 1F, CF₂).

Mass (ESI⁻): 723.3 [M−H]⁻

### Synthesis of Intermediate Compound 11:

A mixture of compound 10 (1 eq., 10.83 g, 11.95 mmol), HATU (1.5 eq., 6.95 g, 17.93 mmol), NH₄Cl (3 eq., 1.92 g, 35.85 mmol) and DIPEA (5.0 eq., 7.72 g, 59.75 mmol) in DMF was stirred at room temperature for 5 h. Brine was added and the mixture was extracted with AcOEt (2×). The combined organic layers were washed with brine (4×), dried over MgSO₄, filtered and concentrated. The crude residue was purified by flash chromatograph (Biotage® 80 g, cyclohexane/AcOEt from 90:10 to 70:30) to afford compound 11 (5.7 g, 66% yield, 93% purity) as a colorless oil.

¹⁹F NMR (CDCl₃, 282.5 MHz): −110.5 (d, 270 Hz, 1F, CF₂); −112.5 (d, 270 Hz, 1F, CF₂).

Mass (ESI⁺): 724.3[M+H]⁺, 746.3[M+Na]⁺, 762.3[M+K]⁺

### Synthesis of Intermediate Compound 16:

NaBH₄ (7 eq., 1.76 g, 46.5 mmol) was added to a solution of compound 9 (1 eq., 5.00 g, 6.64 mmol) in dry THE (11 mL) and MeOH (33 mL) cooled to 0° C. under inert atmosphere. The mixture was then stirred at 25° C. for 2.5 h. As the reaction was not complete, an additional portion of NaBH₄ (7 eq., 1.76 g, 46.5 mmol) was added to the reaction previously cooled to 0° C. The reaction mixture was stirred for an additional 2.5 h at 25° C. After completion of the reaction, a saturated aqueous solution of NH₄Cl and brine where added. The aqueous layer was extracted with AcOEt and the organic layer was separated and washed with brine prior to be dried over Na₂SO₄, filtered and concentrated to afford crude compound 16 (4.41 g, 93%) as an off-white solid. The material was engaged in the next step without purification.

¹⁹F NMR (CDCl₃, 282.5 MHz): −113.3 (ddd, 264 Hz, 14 Hz, 14 Hz, 1F, CF₂); −114.3 (ddd, 264 Hz, 15 Hz, 1F, CF₂)

Mass (ESI⁺): 728.3 [M+H₂O]⁺; 733.3 [M+Na]⁺; 749.2 [M+K]⁺

### Synthesis of Intermediate Compound 17:

A solution of compound 16 (1 eq., 8.00 g, 11.3 mol) in dry DCM (163 mL) was added to a solution of triflic anhydride (2.3 eq., 4.34 mL, 15.9 mmol) and pyridine (2.3 eq., 2.11 mL, 25.9 mmol) in dry DCM (163 mL) cooled to 0° C. under inert atmosphere. The mixture was stirred at 0° C. for 1 h and at room temperature for an additional 2 h. Water was then added to the reaction mixture and the layers were separated. The aqueous layer was extracted with DCM and the combined organic layers were dried over Na₂SO₄, filtered and concentrated to afford crude compound 17 (9.44 g, 100%) as an off-white solid. The material was engaged in the next step without purification.

¹⁹F NMR (CDCl₃, 282.5 MHz): −74.5 (s, 3F, CF₃); −113.8 (ddd, 258 Hz, 23 Hz, 5 Hz, 1F, CF₂); −116.2 (brdd, 258 Hz, 23 Hz, <5 Hz, 1F, CF₂).

Mass (ESI⁺): 860.2 [M+H₂O]⁺; 865.2 [M+Na]⁺; 881.2 [M+K]⁺

### Synthesis of Intermediate Compound 18:

Sodium azide (0.96 g, 14.8 mmol, 5 eq) was added at room temperature to a solution of compound 17 (1 eq., 2.5 g, 2.97 mmol) in dry DMF under inert atmosphere. The reaction mixture was stirred at 50° C. for 7 h prior to be cooled to room temperature. AcOEt was added and the organic mixture was washed with brine (2×), dried over Na₂SO₄, filtered and concentrated. The crude material was purified by flash chromatography (AIT® 80 g SiO₂ cartridge, cyclohexane/ethyl acetate from 100:0 to 80:20) to afford compound 18 (0.42 g, 19%) as a white solid.

¹⁹F NMR (CDCl₃, 282.5 MHz): −111.4 (ddd, 257 Hz, 21 Hz, 10 Hz, 1F, CF₂); −112.52 (ddd, 257 Hz, 22 Hz, 11 Hz, 1F, CF₂).

Mass (ESI⁺): 753.3 [M+H₂O]⁺; 758.3 [M+Na]⁺; 774.3 [M+K]⁺

### Synthesis of Intermediate Compound 12:

Procedure A: From Compound 11

Under inert atmosphere, BH₃.THF complex (6 eq., 1.0M in THF, 43.9 mL, 43.9 mmol) was added to a solution of compound 11 (1 eq., 5.70 g, 7.32 mmol) in dry THE (26.5 mL) at room temperature. The reaction mixture was then refluxed for 18 h. After completion of the reaction, methanol (10 mL) was carefully added at room temperature under stirring and the mixture was refluxed for an additional 30 min prior to be cooled and concentrated. HCl (6M in water, 10 mL) was added and the mixture was heated to reflux for a brief minute and then cooled. The mixture was brought to pH=10 using a saturated aqueous solution of NaHCO₃ and extracted with DCM (3×10 mL). The combined organic layers were dried over Na₂SO₄, filtered and concentrated. The crude residue was purified by flash chromatography (Biotage® ZIP KP-Sil 45 g cartridge, DCM/DCM:MeOH:NH₄OH 80:18:2 v/v/v from 100:0 to 70:30) to afford compound 12 (4.0 g, 77%) as a white solid.

Procedure B: From Compound 18

Under inert atmosphere, lithium aluminium hydride (1M in THF, 2 eq., 1.09 mL, 1.09 mmol) was added to a solution of compound 18 (1 eq., 0.40 g, 0.54 mmol) in dry THE (5.39 mL) previously cooled to 0° C. The reaction mixture was stirred at 0° C. for 2 h. A saturated aqueous solution of Na₂SO₄ was then added and the mixture was allowed to reach gradually room temperature and was stirred for an additional 2 h before being filtered over Celite®. The solid was washed with AcOEt and the organic layer of the filtrate was dried over Na₂SO₄, filtered and concentrated. The crude material was purified by flash chromatography (Biotage® KP-Sil 10 g cartridge, cyclohexane/ethyl acetate 100:0 to 60:40) to afford compound 12 (0.13 g, 33%) in the form of a white solid.

¹⁹Fdec NMR (CDCl₃, 282.5 MHz): −114.5 (d, 254 Hz, 1F, CF₂); −115.4 (d, 254 Hz, 1F, CF₂).

Mass (ESI⁺): 710.3 [M+H]⁺; 732.2 [M+Na]⁺; 748.3 [M+K]⁺

### Synthesis of Intermediate Compound 14:

A solution of compound 12 (1 eq., 300 mg, 0.423 mmol) in DCE (1.7 mL) was added to a solution of compound 13 (obtained from Journal of Organic Chemistry 1998, 63, 3741-3744) (1.1 eq., 160 mg, 0.465 mmol) in DCE (1.7 mL) under inert atmosphere. MgSO₄ (10 eq., 508 mg, 4.23 mmol) was added and the reaction was stirred under reflux for 2 h. The mixture was cooled to 0° C. and then sodium triacetoxyborohydride (2 eq., 184 mg, 0.845 mmol) and acetic acid (1 eq., 28.2 mg, 0.0269 mL, 0.423 mmol) were added and the resulting mixture was stirred at room temperature for 12 h. Water and NaHCO₃ (10% aq) were added to the mixture before it was extracted with AcOEt. The combined organic layers were washed with water, dried over Na₂SO₄, filtered and concentrated. The crude residue was purified by flash chromatography (Biotage® SNAP 10 g, cyclohexane/AcOEt from 95/5 to 80/20) to afford a mixture containing compound 14 (221 mg,) in the form of a white solid.

Mass (ESI⁺): 1039.5 [M+H]⁺; 1061.5 [M+Na]⁺; 1077.5 [M+K]⁺.

### Synthesis of Intermediate Compound 15:

A solution of a mixture containing compound 14 (1 eq., 20 mg, 0.98 mmol) in toluene (0.5 mL) and acetic acid (10 eq., 0.01 mL, 0.19 mmol) was heated at reflux for 7 h. The reaction mixture was concentrated to afford a crude compound 15 in the form of a beige solid.

Mass (ESI⁺): 1029.4 [M+Na]⁺; 1045.4 [M+K]⁺

### Synthesis of Intermediate Compound 19:

Trifluoroacetic acid (5.9 eq., 21.8 μL, 0.29 mmol) was added to a solution of crude compound 15 (1.0 eq, 50.0 mg, 0.05 mmol) in water (2.7 μL) and dichloromethane (109 μL). The reaction was stirred overnight at room temperature. Water was then added and the pH of the solution was adjusted to pH=8-9 with a solution of NaOH (2M in water). The aqueous layer was then extracted 3 times with AcOEt and the combined organic layer was dried over Na₂SO₄, filtered and concentrated to afford crude compound 19 (38.7 mg) as a yellowish solid.

Mass (ESI⁺): 807.4 [M+H]⁺.

### Synthesis of Intermediate Compound 5:

Palladium (loading 10 wt %, support activated carbon, 0.10 eq., 5.1 mg, 0.005 mmol) was added to a solution of crude compound 19 (1 eq., 38.7 mg, 0.05 mmol) in THF (1.9 mL), previously degassed with nitrogen. A solution of HCl (2M in water, 4.0 eq., 0.09 mL, 0.19 mmol) was then added. The mixture was placed under hydrogen atmosphere and was stirred for 16 h. The reaction was degassed with nitrogen prior to be filtered (0.45 μm, H-PTFE) to remove the palladium residues. The filter was washed with water and the filtrate was concentrated to afford crude compound 5 (12 mg).

Mass (ESI⁻): 391.0 [M−H]⁻

### 1.3. Synthesis of Compound 24

Compound 24 can be prepared according to the following synthesis route:

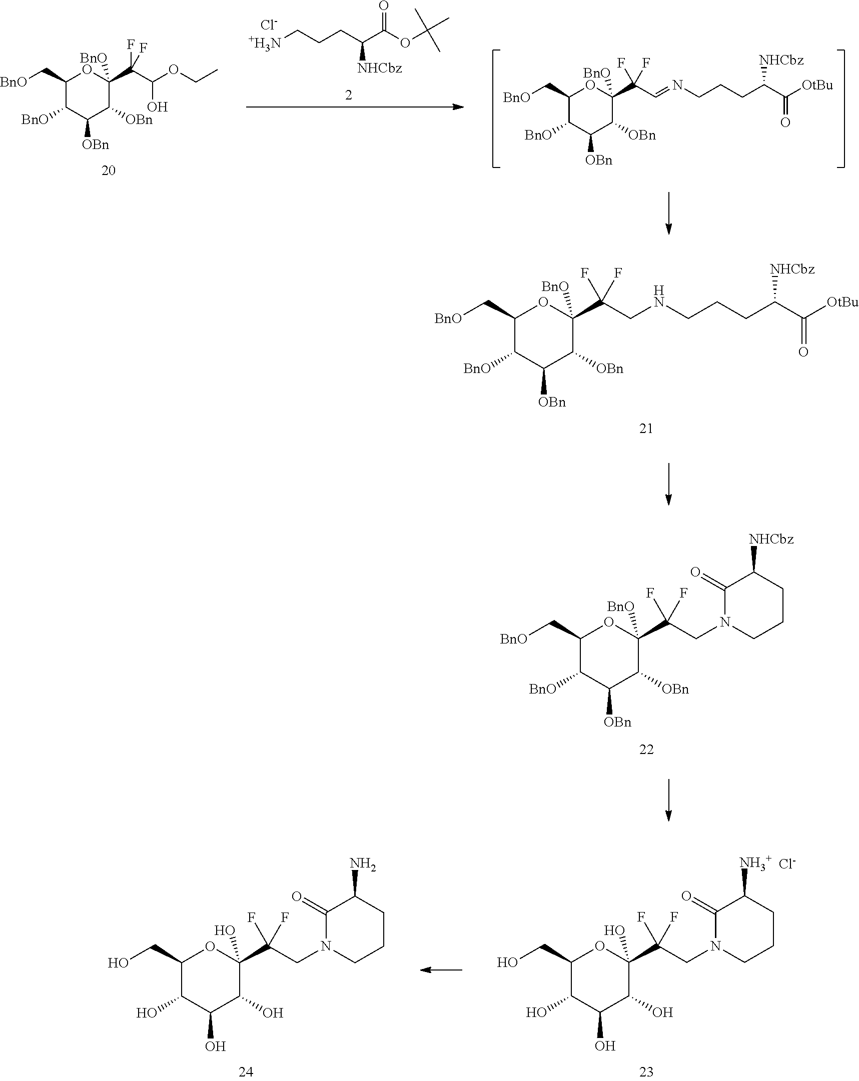

### Synthesis of Intermediate Compound 20:

Compound 20 was prepared following the same protocol than for the preparation of compound 1 and disclosed in WO2015/140178 (cf. compound 2) and applied to a glucose instead of a galactose moiety.

Mass (ESI⁺): 772.3 [M+NH₄]⁺; 777.3 [M+Na]⁺; 793.3 [M+K]⁺

### Synthesis of Intermediate Compound 21:

To a solution of compound 20 (1.2 eq., 3.20 g, 4.24 mmol) in DCE (27 mL) under inert atmosphere was added sequentially PsNEt₂ (3.2 mmol/g supported diethylamine, 3.7 eq., 4.13 g, 13.2 mmol) and MgSO₄ (3 eq., 1.30 g, 10.8 mmol). A solution of compound 2 (1 eq., 2.15 g, 3.53 mmol) in DCE (9.75 mL) was then added and the reaction was then refluxed 18 h. The mixture was cooled to room temperature and then rapidly filtered. The resulting solution was transferred in a round bottom flask and was cooled to 0° C. under inert atmosphere. To this solution were added portionwise sodium triacetoxyborohydride (95%, 2.9 eq., 2.25 g, 10.1 mmol) and acetic acid (1 eq., 0.20 mL, 3.53 mmol). The reaction was stirred at room temperature for 18 hours.

Water, NaHCO₃ (10% aqueous solution) and DCM were added and the mixture was then extracted with DCM three times. Methanol was added and the combined organic layer was dried over Na₂SO₄, filtered and concentrated.

The resulting crude oil was purified by chromatography (Irregular SiO₂ 40-63 μm, cyclohexane/ethyl acetate 95:5 to 75:25) to afford compound 21 (2.8 g, 85% purity, 78% yield) as a colorless oil.

¹⁹Fdec NMR (CDCl₃, 282.5 MHz): −109.3 (d, 258 Hz, 1F, CF₂), −110.3 (d, 258 Hz, 1F, CF₂).

Mass (ESI⁺): 1015.5 [M+H]⁺, 1037.5 [M+Na]⁺, 1053.5 [M+K]⁺

### Synthesis of Intermediate Compound 22:

In a sealed tube, a solution of compound 21 (1 eq., 85% purity, 2.80 g, 2.34 mmol) in toluene (26 mL) and acetic acid (10 eq., 1.34 mL, 23.4 mmol) was heated at reflux for 18 h. The reaction mixture was concentrated. The residue was purified by flash chromatography (80 g irregular SiO₂ 40-63 μm, cyclohexane/ethyl acetate 95:5 to 75:25) to afford compound 22 (2.43 g, 80% purity, 100%).

¹⁹F NMR (CDCl₃, 282.5 MHz): −107.7 (brdd, 257 Hz, 30 Hz, 1F, CF₂), −110.8 (brdd, 258 Hz, 26 Hz, 1F, CF₂).

Mass (ESI⁺): 963.3 [M+Na]⁺, 979.3 [M+K]⁺

### Synthesis of Intermediate Compound 23:

Palladium (loading 10 wt. %, support activated carbon, 0.22 g, 0.21 mmol, 0.1 eq) was added to a solution of compound 22 (80% purity, 2.43 g, 2.07 mmol, 1 eq) in THE (42 mL), previously degassed with nitrogen. A solution of HCl (2M in water, 4.1 mL, 8.2 6 mmol, 4 eq) was then added. The mixture was placed under hydrogen atmosphere and was stirred for 18 h. The reaction was degassed with nitrogen prior to be filtered (0.20 μm, Polyamide) to remove the palladium residues. The filter was washed with a mixture of THE and water and the filtrate was concentrated to remove the THF. The residue was then diluted with water and the solution was filtered (0.2 μm, H-PTFE) before being freeze dried to afford compound 23 (0.90 g, 90% purity, 100% yield) as a white foam.

¹⁹FNMR (D₂O, 282.5 MHz): −115.3 (ddd, 251 Hz, 26 Hz, 8 Hz, 1F, CF₂), −116.8 (ddd, 251 Hz, 26 Hz, 8 Hz, 1F, CF₂).

Mass (ESI⁺): 357.1 [M+H]⁺ (NH₂ form)

### Synthesis of Compound 24:

Compound 23 (90% purity, 0.90 g, 2.06 mmol) was dissolved in a minimum volume of water. The solution was placed at the top of a small column filled with resin (DOWEX® 50Wx8 previously washed with water). Water was first used as eluent to remove impurities and then a solution of aqueous ammonia (0.1M NH₄OH) was used to elute the desired compound from the resin. The solution of compound 24 was then freeze-dried to afford pure compound 24 (630 mg, 86% yield).

¹⁹FNMR (D₂O, 282.5 MHz): −115.2 (ddd, 251 Hz, 21 Hz, 13 Hz, 1F, CF₂), −116.4 (ddd, 251 Hz, 21 Hz, 13 Hz, 1F, CF₂).

Mass (ESI⁺): 357.1 [M+H]⁺, 379.1 [M+Na]⁺, 395.1 [M+K]⁺

### 1.4. Synthesis of Compound 29

Compound 29 was prepared according to the following synthesis route:

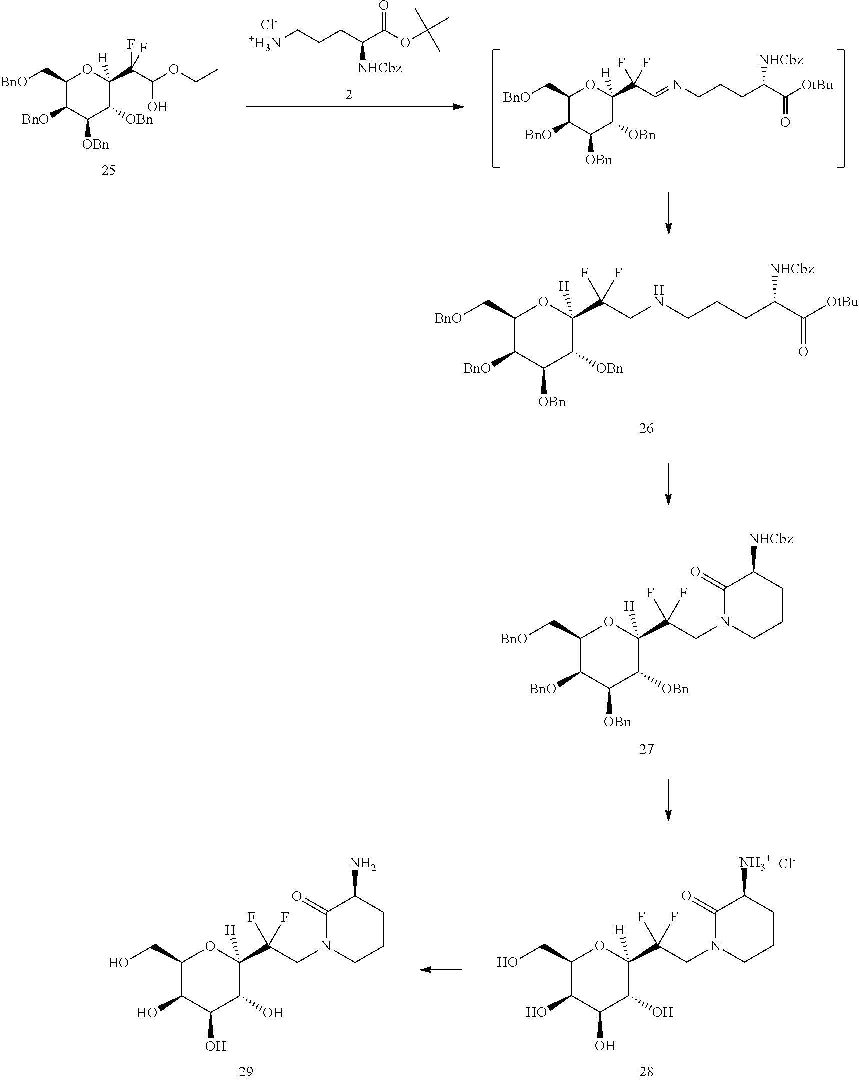

### Synthesis of Compound 25

The synthesis of compound 25 was disclosed in WO2012085221 (cf. compound 2p).

### Synthesis of Intermediate Compound 26:

To a solution of compound 25 (1.2 eq., 2.75 g, 4.24 mmol) in DCE (27 mL) under inert atmosphere was added sequentially PsNEt₂ (3.2 mmol/g supported diethylamine, 3.7 eq., 4.13 g, 13.2 mmol) and MgSO₄ (3 eq., 1.30 g, 10.8 mmol). A solution of compound 2 (1 eq., 2.15 g, 3.53 mmol) in DCE (9.75 mL) was then added and the reaction was then refluxed 18 h. The mixture was cooled to room temperature and then rapidly filtered. The resulting solution was transferred in a round bottom flask and was cooled to 0° C. under inert atmosphere. To this solution were added portionwise sodium triacetoxyborohydride (2.9 eq., 2.25 g, 10.6 mmol) and acetic acid (1 eq., 0.20 mL, 3.53 mmol). The reaction was stirred at room temperature for 2 hours.

Water, NaHCO₃ (10% aqueous solution) and DCM were added and the mixture was then extracted with DCM three times. Methanol was added and the combined organic layer was dried over Na₂SO₄, filtered and concentrated.

The resulting crude oil was purified by chromatography (Irregular SiO₂ 40-63 μm, cyclohexane/ethyl acetate 95:5 to 75:25) to afford compound 26 (2.3 g, 72% yield) as a colorless oil.

¹⁹Fdec NMR (CDCl₃, 282.5 MHz): −108.6 (dddd, 255 Hz, 35 Hz, 18 Hz, 7 Hz, 1F, CF₂), −112.8 (dm, 255 Hz, 1F, CF₂).

Mass (ESI⁺): 909.4[M+H]⁺, 931 [M+Na]⁺, 947 [M+K]⁺

### Synthesis of Intermediate Compound 27:

In a sealed tube, a solution of compound 26 (1 eq., 2.51 g, 2.76 mmol) in toluene (30 mL) and acetic acid (10 eq., 1.58 mL, 27.6 mmol) was heated under reflux for 18 h. The reaction mixture was concentrated. The residue was purified by flash chromatography (120 g irregular SiO₂, cyclohexane/EtOAc 95:5 to 50:50). At this stage a mixture of compounds 26 and 27 (1.84 g) was obtained. Part of this mixture (140 mg) was dissolved again in toluene (3 mL) and acetic acid (0.1 mL). The mixture was heated under reflux for 16 h. The reaction was concentrated to afford only the desired compound 27 (130 mg).

¹⁹F NMR (CDCl₃, 282.5 MHz): −107.2 (dm, 255 Hz, 1F, CF₂), −111.5 (dm, 255 Hz, 1F, CF₂).

¹⁹F dec NMR (CDCl₃, 282.5 MHz): 107.2 (d, 255 Hz, 1F, CF₂), −111.5 (d, 255 Hz, 1F, CF₂).

Mass (ESI⁺): 835.3 [M+H]⁺, 857.3 [M+Na]⁺, 873.3 [M+K]⁺

### Synthesis of Intermediate Compound 28:

Palladium (loading 10 wt. %, support activated carbon, 17.8 mg, 17 μmol, 0.10 eq) was added to a solution of compound 27 (140 mg, 0.17 mmol, 1 eq) in THE (3.43 mL), previously degassed with nitrogen. A solution of HCl (2M in water, 0.34 mL, 0.67 mmol, 4 eq) was then added. The mixture was placed under hydrogen atmosphere and was stirred for 18 h. The reaction was degassed with nitrogen prior to be filtered (0.45 μm, Polyamide) to remove the palladium residues. The filter was washed with a mixture of THE and water and the filtrate was concentrated to remove the THF. The residue was then diluted with water and the solution was filtered (0.2 μm, H-PTFE) before being freeze dried to afford compound 28 (40 mg, 63%) as a white powder.

¹⁹FNMR (MeOD, 282.5 MHz): −103.1 (dm, 258 Hz, 1F, CF₂), −109.0 (dm, 258 Hz, 1F, CF₂).

¹⁹Fdec NMR (MeOD, 282.5 MHz): −103.1 (d, 258 Hz, 1F, CF₂), −109.0 (d, 258 Hz, 1F, CF₂).

Mass (ESI⁺): 341.1 [M+H]⁺, 363.1 [M+Na]⁺, 379.1 [M+K]⁺ (NH₂ form).

### Synthesis of Compound 29:

Compound 28 (40 mg, 0.11 mmol) was dissolved in a minimum volume of water. The solution was placed at the top of a small column filled with resin (DOWEX® 50Wx8 previously washed with water). Water was first used as eluent to remove impurities and then a solution of aqueous ammonia (0.1M NH₄OH) was used to elute the desired compound from the resin. The solution of compound 29 was then freeze-dried to afford pure compound 29 (21 mg, 58% yield).

¹⁹FNMR (D₂O, 282.5 MHz): −107.8 (ddd, 255 Hz, 11 Hz, 7 Hz, 1F, CF₂), −113.6 (dm, 255 Hz, 1F, CF₂).

¹⁹Fdec NMR (D₂O, 282.5 MHz): −107.8 (d, 255 Hz, 1F, CF₂), −113.6 (d, 25 5 Hz, 1F, CF₂).

Mass (ESI⁻): 339.2 [M−H]⁻, 361.1 [M+Na−2H]⁻, 375.1 [M+Cl]⁻

### 1.5. Synthesis of Compound 34

Compound 34 can be prepared according to the following synthesis route:

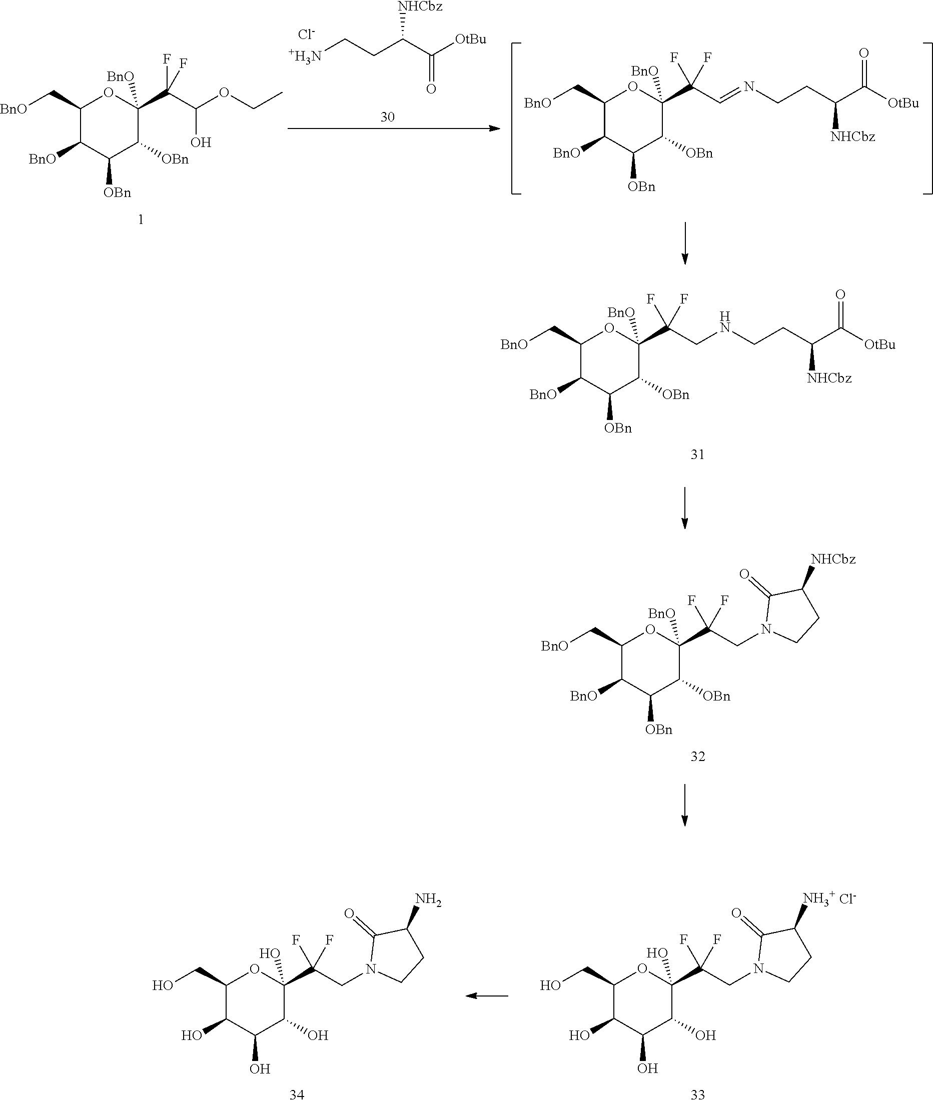

### Synthesis of Intermediate Compound 30:

Compound 30 is prepared according to the following two steps:

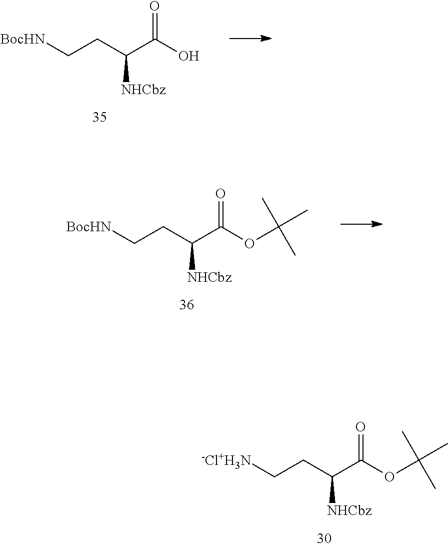

Compound 36 is prepared from commercially available compound 35 according to the method disclosed in Organic Letters 2006, 8, 17, 3865-3868 (supporting information page 17).

Compound 30 is then obtained from compound 36 according to a protocol disclosed in J. Org. Chem. 1994, Vol. 59, No. 11, 3216-3218 as follows.

Compound 36 (1.0 eq., 1.8 g, 3.61 mmol) was dissolved in a solution of HCl (1 M in AcOEt, 2.5 eq., 9.03 mL, 9.03 mmol). The reaction mixture was stirred at room temperature for 3 h. HCl (1 M in AcOEt, 2.5 eq., 9.03 mL, 9.03 mmol) was added again to complete the reaction. The reaction was stirred at room temperature for an additional 18 h and was then concentrated and co-evaporated with diethyl ether to afford 1.2 g of compound 30 (60% purity, 58% yield). The material was engaged in the next step without purification.

¹H NMR (MeOD, 300 MHz): 1.44 (s, 9H), 2.04 (m, 1H), 2.20 (m, 1H), 3.02 (t, 7.2 Hz, 2H), 4.17 (dd, 9.3 Hz, 5.1 Hz, 1H), 5.12 (s, 3H), 7.33-7.36 (m, 5H).

Mass (ESI⁺): 309.2 [M+H]⁺ (NH₂ form)

### Synthesis of Intermediate Compound 31:

To a solution of compound 1 (2.0 eq., 3.15 g, 4.18 mmol) in DCE (16 mL) under inert atmosphere was added sequentially added PsNEt₂ (3.2 mmol/g supported diethylamine, 3.1 eq., 2 g, 6.4 mmol) and MgSO₄ (3 eq., 1.30 g, 10.8 mmol). A solution of compound 30 (1.0 eq., 60% purity, 1.2 g, 2.09 mmol) in DCE (6 mL) was then added and the reaction was refluxed for 18 h. The mixture was cooled to room temperature and then rapidly filtered. The resulting solution was transferred in a round bottom flask and was cooled to 0° C. under inert atmosphere. To this solution were added portionwise sodium triacetoxyborohydride (5.0 eq., 2.21 g, 10.4 mmol) and acetic acid (1.0 eq., 0.12 mL, 2.09 mmol). The reaction was stirred at room temperature for 2 hours.

Water, sodium bicarbonate (10% aqueous solution) and DCM were added and the mixture was then extracted with DCM (3×). Methanol was added and the combined organic layer was dried over sodium sulfate, filtered and concentrated.

The resulting crude oil was purified by chromatography (80 g irregular SiO₂ 40-63 μm, cyclohexane/ethyl acetate 95:5 to 70:30) to afford compound 31 (1.35 g, 66% yield) as a colorless oil.

¹⁹Fdec NMR (CDCl₃, 282.5 MHz): −109.7 (d, 258 Hz, 1F, CF₂), −110.7 (d, 258 Hz, 1F, CF₂).

Mass (ESI⁺): 1001.5 [M+H]⁺, 1039.5 [M+K]⁺

### Synthesis of Intermediate Compound 32:

A solution of compound 31 (1 eq., 86% purity, 1.35 g, 1.16 mmol) in toluene (13 mL) and acetic acid (10 eq., 0.66 mL, 11.6 mmol) under inert atmosphere was heated under reflux for 18 h. Water, a solution of sodium bicarbonate (10% in water) and ethyl acetate were added. The aqueous layer was extracted with ethyl acetate (×3). The combined organic layers were washed with brine, dried over sodium sulfate, filtered and concentrated. The residue was purified by flash chromatography (40 g irregular SiO₂, cyclohexane/EtOAc 90:10 to 80:20) to afford compound 32 (960 mg, 91% purity, 72% yield).

¹⁹F dec NMR (CDCl₃, 282.5 MHz): −107.7 (d, 259 Hz, 1F, CF₂), −108.6 (d, 259 Hz, 1F, CF₂).

Mass (ESI⁺): 927.4 [M+H]⁺, 944.5 [M+NH₄]⁺, 949.4 [M+Na]⁺, 965.4 [M+K]⁺

### Synthesis of Intermediate Compound 33:

Palladium (loading 10 wt. %, support activated carbon, 0.13 g, 0.12 mmol, 0.10 eq) was added to a solution of compound 32 (1.25 g, 91% purity, 1.23 mmol, 1.0 eq) in THE (25 mL) previously degassed with nitrogen. A solution of HCl (2M in water, 2.45 mL, 4.9 mmol, 4.0 eq) was then added. The mixture was placed under hydrogen atmosphere and was stirred for 2 days. The reaction was degassed with nitrogen prior to be filtered (0.45 μm, Polyamide) to remove the palladium residues. The filter was washed with a mixture of THE and water and the combined solution was concentrated to remove the THF. The residue was then diluted with water and the solution was filtered (0.2 μm, H-PTFE) before being freeze dried to afford compound 33 (0.55 g, 85% purity, 100% yield). Compound 33 is in the form of two tautomers as follows:

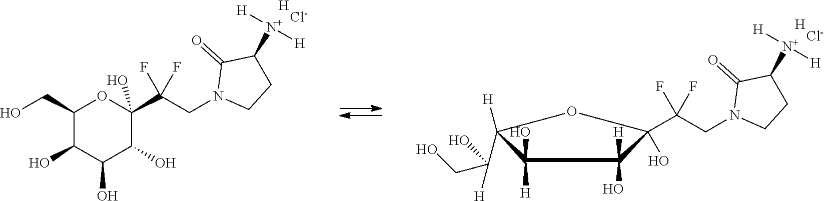

¹⁹Fdec NMR (MeOD, 282.5 MHz): 2 tautomer forms with a ratio of 80:20

Major form: −117.5 (d, 257 Hz, 1F, CF₂), −118.4 (d, 257 Hz, 1F, CF₂).

Minor form: −115.2 (d, 253 Hz, 1F, CF₂), −116.9 (d, 253 Hz, 1F, CF₂).

Mass (ESI⁺): 343.1 [M+H]⁺, 365.1 [M+Na]⁺, 381.1 [M+K]⁺

### Synthesis of Compound 34:

Compound 33 (550 mg, 1.23 mmol) was dissolved in a minimum volume of water. The solution was placed at the top of a small column filled with resin (1.5 g, DOWEX® 50Wx8 previously washed with water). Water was first used as eluent to remove impurities and then a solution of aqueous ammonia (0.1M NH₄OH) was used to elute the desired compound from the resin. The solution of compound 34 was filtered (0.2 μm, H-PTFE) then freeze-dried to afford pure compound 34 (240 mg, 67% yield). Compound 34 is in the form of two tautomers as follows:

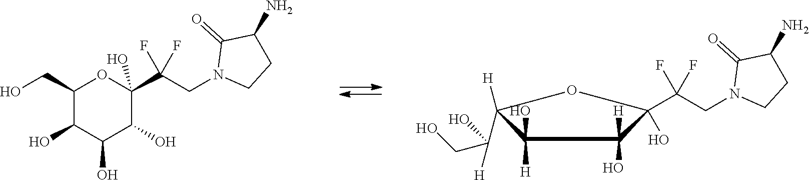

¹⁹Fdec NMR (D₂O, 282.5 MHz): 2 tautomer forms with a ratio of 56:44

Major form: −116.6 (d, 253 Hz, 1F, CF₂), −117.5 (d, 253 Hz, 1F, CF₂).

Minor form: −114.9 (d, 251 Hz, 1F, CF₂), −116.6 (d, 251 Hz, 1F, CF₂).

Mass (ESI+): 343.1 [M+H]⁺, 365.1 [M+Na]⁺, 381.1 [M+K]⁺

### 2. Biological Activity:

2.1. Effects of Compound 6 on Gene Expression in Human Dermal Fibroblasts. Human «Full Transcriptome» Analysis Using Affymetrix Microarray

In the present study, the transcriptional effects (modulation of gene expression) of compound 6 were evaluated on normal human dermal fibroblasts (NHDF) under basal conditions.

More specifically, the comparative analysis of the different transcriptomic profiles was performed using an Affymetrix GeneAtlas platform and the human “full transcriptome” U219 chip, which includes 36,000 transcripts and variants.

### Materials and Methods

Normal human dermal fibroblasts (NHDF) were grown with Dulbecco's Modified Eagle Medium (DMEM) supplemented with Fetal Calf Serum (FCS) 10%, antibiotics (Penicillin 50 U/ml—Streptomycin 50 μg/ml) and L-Glutamine 2 mM final. Cells were grown in 37° C. and 5% CO₂ incubator.

### Gene Screening Assay

Fibroblasts were seeded in 48-well plates and cultured for 24 hours in culture medium and in assay medium for a further 24 h. The medium was then replaced by assay medium containing or not (control) the test compound at different concentrations for 48 hours. All experimental conditions were performed in triplicate. At the end of incubation, the culture supernatants were removed and the cells were washed in a phosphate buffered saline (PBS) solution and immediately frozen at −80° C.

### Differential Expression Analysis

Before RNA extraction, the replicates were pooled. Total RNA was extracted from each sample using TriPure Isolation Reagent® according to the supplier's instructions. The amount and quality of total RNA were evaluated for all samples using capillary electrophoresis (Bioanalyzer 2100, Agilent technologies). From each RNA, a labeled and amplified anti-sens RNA (aRNA) was obtained using GeneChip 3′IVT PLUS Kit (Affymetrix). For each labeled and amplified aRNA sample the profiles were evaluated before and after fragmentation using capillary electrophoresis (Bioanalyzer 2100, Agilent technologies). Hybridization of fragmented aRNA onto Affymetrix® U219 chip (36,000 transcripts and variants) was performed in the GeneAtlas™ fluidics Affymetrix® hybridization station for 20 hours at 45° C. U219 chip was analyzed using the GeneAtlas™ Imaging station (Affymetrix®—resolution 2 μm) to generate fluorescence intensity data.

### Data Management and Result Presentation

Expression Console and Quality controls: Data were normalized with the Expression Console (Affymetrix®) software using RMA algorithm. Then a quality control of the labeling and the hybridization was performed. Hybridization and labeling steps successfully passed the quality controls for these experiments.
Data reduction, Excel file description: Once normalized with Expression Console, data were transferred into a Microsoft Excel® file in order to go further into data reduction. Calculation and tools were added in order to rank and sort data, and finally to support data interpretation. Detection thresholds in terms of fold change were defined and applied on normalized data.

| | Arbitrary classification |
|---|---|
| Fold Change | of observed effects |
| ≥2 | Upregulated probes (UP) |
| ≤0.5 | Downregulated probes (DR) |

Results are considered and presented per gene (and not probe). A probe set is a collection of probes designed to interrogate a given sequence of a gene. For data interpretation, the most important relative expression value obtained with one probe is considered to be representative of the corresponding gene.
The file contains the following data:
   Relative expression (RE) for each sample,
   Fold change calculation,
   Gene information.
Identification of the biological processes involved: The list of significantly modulated genes was transferred in the online database DAVID (Database for Annotations, Visualization and Integrative Discovery: http://david.abcc.ncifcrf.gov/) for a functional analysis (Genome Biology 2007, 8: R183, Nucleic Acids Research, 2009, Vol. 37, No. 1 1-13). Gene Ontology database has been more specifically used for the data interpretation. DAVID functional annotation part was used to cluster modulated genes into significant biological processes. This analysis does not take into account the trend (UR or DR) or the signal intensity but only identifies the biological functions implicated in the comparison of interest. DAVID database uses the Gene Ontology consortium (http://www.geneontology.org) vocabularies (GO terms) to describe gene products in terms of their associated biological processes. Among them, only biological processes with p-value ≤0.05 were taken into account.
Signal transduction pathway analsis: The results were then processed with IPA (Ingenuity Pathway Analysis, Qiagen®) software to identify signal transduction pathways modulated by each treatment. This software takes into account the Fold Change values of each gene and, when there is enough information, the direction of modulation of the signal transduction pathways can be identified. The relevance of the effect of each treatment on a given pathway was quantified by z-score. The z-score predicts the directional change on that effect.

| | Predicted |
|---|---|
| z-score | Activation State |
| >0 | Increased |
| <0 | Decreased |

### Results

### Identification of Biological Process Involved

The gene modulations of NHDF treated with compound 6 (2 mg/ml) vs control were analyzed to cluster modulated genes into significant biological processes (p-value ≤0.05).

Table 1 below shows that the main biological processes involved with test compound 6, are:
the lipid metabolic process and the cholesterol biosynthetic and metabolic process;
the extracellular matrix organization;
the wound healing and response to wounding;
the oxidation-reduction process.

TABLE 1
Identification of the biological processes involved
in NHDF and stimulated by compound 6 (2 mg/ml)
Test compound 6 (2 mg/ml) versus Control

| Term | Count | % | p-value | Genes |
|---|---|---|---|---|
| Cholesterol | 15 | 0.01072003 | 1.17E−09 | TM7SF2, EBP, MVD, |
| biosynthetic | | | | CYP51A1, HMGCR, |
| process | | | | HMGCS1, FDPS, LSS, |
| | | | | ACLY, G6PD, DHCR7, |
| | | | | INSIG1, IDI1, HSD17B7, |
| | | | | NSDHL |
| Lipid | 22 | 0.01572270 | 2.39E−05 | SREBF1, CHKA, SC5D, |
| metabolic | | | | LIPA, PLA2G15, LDLR, |
| process | | | | FADS1, ABHD4, |
| | | | | HMGCS1, FADS2, |
| | | | | ABHD3, ACLY, PLPP1, |
| | | | | GPCPD1, ASAH1, APOL2, |
| | | | | G6PD, PTGDS, TPP1, |
| | | | | SRD5A3, MGLL, LRP8 |
| Cholesterol | 13 | 0.00929069 | 9.34E−05 | SOAT1, SREBF1, EBP, |
| metabolic | | | | TNFSF4, LDLR, LEPR, |
| process | | | | ABCA1, APOL2, NPC1, |
| | | | | APOL1, NPC2, INSIG1, |
| | | | | PCSK9 |
| Oxidation- | 54 | 0.0385921 | 1.26E−05 | TM7SF2, SC5D, PDIA3, |
| reduction | | | | HMGCR, PGD, AKR1C3, |
| process | | | | AKR1C2, TDO2, PTGIS, |
| | | | | PLOD2, CREG1, P4HA3, |
| | | | | SRD5A3, LOXL4, LOX, |
| | | | | LOXL2, LOXL1, |
| | | | | SH3PXD2B, POR, DHRS7, |
| | | | | DHRS3, G6PD, CYP27A1, |
| | | | | PRDX6, CYBRD1, KDSR, |
| | | | | TXNRD1, STEAP2, |
| | | | | STEAP1, ME1, HSD3B7, |
| | | | | HSD17B14, CYP51A1, |
| | | | | HSD17B12, FTH1, P3H2, |
| | | | | ALDH1A3, DHCR7, |
| | | | | CYP26B1, FASN, GSTO1, |
| | | | | HSD17B7, NSDHL, |
| | | | | CYP19A1, BMP2, FADS1, |
| | | | | SCD, FOXRED2, FADS2, |
| | | | | AAED1, SOD2, CYBA, |
| | | | | AKR1B1, PHGDH |
| Extracellular | 33 | 0.02358406 | 1.46E−09 | TNC, HSD17B12, |
| matrix | | | | COL3A1, ELN, ITGA11, |
| organization | | | | ITGA10, POSTN, DCN, |
| | | | | VIT, SOX9, TNFRSF11B, |
| | | | | CD44, ITGB8, LOX, FBN2, |
| | | | | COL8A1, COL8A2, |
| | | | | LOXL1, SPP1, OLFML2B, |
| | | | | FBN1, HSPG2, CCDC80, |
| | | | | ITGA2, ECM2, COL5A2, |
| | | | | COL5A1, LAMA1, |
| | | | | LAMA4, COL14A1, |
| | | | | ITGA8, VCAN, MFAP5 |
| Response to | 10 | 0.00714669 | 0.0032146 | SLC1A2, SLC1A3, CCL2, |
| wounding | | | | SULF2, TNC, AURKA, |
| | | | | ABHD2, ID3, DST, GAP43 |
| Wound | 11 | 0.00786135 | 0.0052167 | WNT5A, DCBLD2, IL6, |
| healing | | | | NOG, TNC, COL3A1, |
| | | | | SMAD3, LOX, DCN, IL24, |
| | | | | NRG1 |

Modulation of the mRNA Expression

Tables 2, 3, 4, 5 below present the different genes involved respectively in the lipid synthesis, the metabolism of cholesterol, the synthesis of cholesterol and the differentiation of adipocytes, which were modulated by the tested compound 6. The fold change expresses if they are upregulated (≥2) or down regulated (≤0.5).

Tables 6, 7, 8 below present the different genes involved respectively in the fibrogenesis, the tensile strength of skin and the synthesis of reactive oxygen species (ROS), which were modulated by the tested compound 6. The fold change expresses if they are upregulated (≥2) or down regulated (≤0.5).

Tables 9, 10 below present the different genes involved respectively in the inflammatory response and the chronic inflammatory disorder, which were modulated by the tested compound 6. The fold change expresses if they are upregulated (≥2) or down regulated (≥0.5).

TABLE 2
Table of the set of genes involved in the lipid synthesis
in NHDF and modulated by compound 6 (2 mg/ml)

| Affy U219 | Control | Compound 6 (2 mg/ml) | | Gene |
|---|---|---|---|---|
| Probe Set ID | REadj1 | REadj2 | Fold change | Symbol |
| 11739503_at | 191.50 | 984.71 | 5.14 | ABCA1 |
| 11720859_s_at | 41.51 | 94.68 | 2.28 | ABHD3 |
| 11744255_a_at | 1226.17 | 473.16 | 0.39 | ACADVL |
| 11720620_s_at | 469.81 | 1051.41 | 2.24 | ACLY |
| 11743606_a_at | 687.27 | 1627.23 | 2.37 | ACSL3 |
| 11753466_a_at | 47.54 | 297.29 | 6.25 | ACSS2 |
| 11725176_s_at | 160.10 | 45.20 | 0.28 | AGTR1 |
| 11751921_s_at | 929.38 | 398.39 | 0.43 | AHR |
| 11715430_a_at | 916.68 | 2005.30 | 2.19 | AKR1B1 |
| 11729101_a_at | 1496.04 | 301.44 | 0.20 | AKR1C2 |
| 11715711_a_at | 1628.04 | 539.24 | 0.33 | AKR1C3 |
| 11754184_a_at | 357.00 | 52.16 | 0.15 | ALDH1A3 |
| 11726692_at | 576.71 | 50.51 | 0.09 | ANGPT1 |
| 11755151_a_at | 239.40 | 116.31 | 0.49 | ARNTL |
| 11743547_a_at | 479.98 | 1612.42 | 3.36 | ASAH1 |
| 11731841_a_at | 44.73 | 107.38 | 2.40 | B4GALT6 |
| 11753244_a_at | 288.86 | 111.61 | 0.39 | BDNF |
| 11725983_at | 63.94 | 360.80 | 5.64 | BHLHE40 |
| 11743498_at | 21.50 | 61.65 | 2.87 | BMP2 |
| 11716384_at | 381.29 | 89.22 | 0.23 | CCL2 |
| 11763855_x_at | 216.32 | 1014.00 | 4.69 | CD9 |
| 11743077_s_at | 859.60 | 2004.45 | 2.33 | CEBPB |
| 11728000_a_at | 24.72 | 51.50 | 2.08 | CERS1 /// GDF1 |
| 11736285_a_at | 54.36 | 195.43 | 3.60 | CHKA |
| 11744874_x_at | 52.01 | 133.00 | 2.56 | CLN3 |
| 11715524_a_at | 509.77 | 245.44 | 0.48 | COTL1 |
| 11715729_s_at | 20.00 | 51.79 | 2.59 | CYP19A1 |
| 11722564_at | 971.84 | 2431.06 | 2.50 | CYP51A1 /// LRRD1 |
| 11759050_at | 80.18 | 29.27 | 0.37 | DAB1 |
| 11754122_x_at | 1025.64 | 2452.25 | 2.39 | DBI |
| 11743808_a_at | 372.14 | 1520.06 | 4.08 | DHCR7 |
| 11752995_a_at | 55.25 | 115.10 | 2.08 | DLAT |
| 11726800_at | 344.87 | 98.61 | 0.29 | EBF1 |
| 11717859_a_at | 406.76 | 1116.25 | 2.74 | EBP |
| 11719488_at | 47.40 | 275.41 | 5.81 | EDNRA |
| 11752940_a_at | 762.43 | 300.02 | 0.39 | EGR1 |
| 11739910_a_at | 69.22 | 202.15 | 2.92 | ELOVL6 |
| 11748964_a_at | 43.83 | 114.94 | 2.62 | ETV1 |
| 11752670_a_at | 848.70 | 4121.66 | 4.86 | FADS1 |
| 11744899_a_at | 119.94 | 411.52 | 3.43 | FADS2 |
| 11743314_a_at | 143.48 | 371.52 | 2.59 | FASN |
| 11758249_s_at | 1568.01 | 3321.26 | 2.12 | FDPS |
| 11734659_a_at | 114.05 | 54.42 | 0.48 | FOS |
| 11724478_s_at | 52.34 | 135.32 | 2.59 | FOSL1 |
| 11752486_a_at | 70.66 | 148.29 | 2.10 | G6PD |
| 11750975_x_at | 107.23 | 352.51 | 3.29 | GBA |
| 11751085_a_at | 93.74 | 408.42 | 4.36 | GBA /// GBAP1 |
| 11716665_s_at | 52.56 | 382.63 | 7.28 | GDF15 |
| 11734201_s_at | 20.00 | 253.54 | 12.68 | GK |
| 11750247_x_at | 109.90 | 51.42 | 0.47 | GPER1 |
| 11756874_a_at | 103.55 | 20.00 | 0.19 | GRP |
| 11729887_at | 256.73 | 588.45 | 2.29 | HACD1 |
| 11727375_a_at | 323.31 | 896.61 | 2.77 | HMGCR |
| 11716987_a_at | 536.29 | 1779.25 | 3.32 | HMGCS1 |
| 11753445_a_at | 46.17 | 793.59 | 17.19 | HMOX1 |
| 11757575_x_at | 30.55 | 115.49 | 3.78 | HSD17B14 |
| 11732374_x_at | 68.38 | 225.29 | 3.29 | HSD17B7 |
| 11741502_a_at | 37.45 | 86.47 | 2.31 | HSD3B7 |
| 11725931_at | 165.83 | 68.73 | 0.41 | HSPA5 |
| 11728104_at | 23.75 | 97.37 | 4.10 | HTR2B |
| 11744474_s_at | 978.39 | 2141.79 | 2.19 | IDI1 |
| 11740881_x_at | 143.71 | 49.92 | 0.35 | IL15 |
| 11731834_a_at | 21.95 | 53.12 | 2.42 | IL24 |
| 11746463_a_at | 35.02 | 71.08 | 2.03 | IL6 |
| 11716339_a_at | 450.00 | 1800.90 | 4.00 | INSIG1 |
| 11751627_a_at | 164.00 | 377.98 | 2.30 | KDSR |
| 11727145_s_at | 91.74 | 216.98 | 2.37 | KLF11 |
| 11719634_a_at | 466.86 | 173.54 | 0.37 | KLF4 |
| 11746974_a_at | 150.81 | 452.83 | 3.00 | LDLR |
| 11738813_a_at | 81.09 | 242.77 | 2.99 | LEPR |
| 11750566_a_at | 132.38 | 393.27 | 2.97 | LPIN1 |
| 11731324_a_at | 127.93 | 359.16 | 2.81 | LSS |
| 11750913_a_at | 322.82 | 967.16 | 3.00 | ME1 |
| 11753715_a_at | 53.16 | 113.62 | 2.14 | MGST2 |
| 11745767_a_at | 835.50 | 1712.63 | 2.05 | MMP2 |
| 11729695_a_at | 191.37 | 658.20 | 3.44 | MVD |
| 11743010_at | 95.38 | 384.87 | 4.04 | NFIL3 |
| 11729937_at | 371.06 | 59.78 | 0.16 | NGF |
| 11743916_a_at | 63.03 | 483.98 | 7.68 | NPC1 |
| 11745902_a_at | 1803.41 | 3990.87 | 2.21 | NPC2 |
| 11717994_a_at | 64.72 | 146.23 | 2.26 | NR4A1 |
| 11729058_s_at | 21.62 | 158.35 | 7.33 | NR4A3 |
| 11742478_a_at | 123.54 | 40.79 | 0.33 | NRG1 |
| 11720522_a_at | 252.77 | 518.82 | 2.05 | NSDHL |
| 11740559_a_at | 94.47 | 193.78 | 2.05 | NSMAF |
| 11722015_at | 45.39 | 21.38 | 0.47 | OLR1 |
| 11747322_s_at | 79.66 | 276.07 | 3.47 | PCYT2 |
| 11720579_a_at | 672.45 | 122.77 | 0.18 | PDE5A |
| 11743440_at | 216.68 | 106.70 | 0.49 | PDIA3 |
| 11756171_a_at | 65.19 | 189.67 | 2.91 | PFKFB2 |
| 11723205_a_at | 263.62 | 763.60 | 2.90 | PGD |
| 11732188_at | 278.15 | 558.97 | 2.01 | PI4K2A |
| 11726548_at | 111.70 | 48.85 | 0.44 | PITPNM3 |
| 11730106_a_at | 358.00 | 145.11 | 0.41 | PLCB1 |
| 11746264_a_at | 1874.34 | 851.28 | 0.45 | PLPP1 |
| 11734720_a_at | 24.83 | 62.05 | 2.50 | PLPP2 |
| 11716114_x_at | 112.82 | 248.72 | 2.20 | POR |
| 11742107_a_at | 129.02 | 377.32 | 2.92 | PPT1 |
| 11718379_x_at | 242.10 | 85.46 | 0.35 | PRKAG2 |
| 11736245_a_at | 123.87 | 57.47 | 0.46 | PRKAG2 |
| 11756587_a_at | 431.73 | 2172.46 | 5.03 | PTGDS |
| 11732550_at | 152.04 | 59.34 | 0.39 | PTGER2 |
| 11724441_x_at | 604.04 | 108.89 | 0.18 | PTGIS |
| 11718157_s_at | 4499.29 | 1245.04 | 0.28 | PTX3 |
| 11753165_a_at | 68.09 | 148.94 | 2.19 | RAB27A |
| 11715757_a_at | 1808.94 | 4014.23 | 2.22 | RGS2 |
| 11750154_a_at | 228.91 | 1263.63 | 5.52 | RGS3 |
| 11736796_a_at | 281.49 | 92.83 | 0.33 | RUNX1T1 |
| 11725905_a_at | 1072.97 | 216.19 | 0.20 | S1PR3 |
| 11757184_a_at | 260.48 | 611.01 | 2.35 | SC5D |
| 11715563_s_at | 116.60 | 1237.96 | 10.62 | SCD |
| 11730390_at | 454.02 | 223.22 | 0.49 | SEMA3A |
| 11748053_x_at | 117.28 | 500.42 | 4.27 | SERINC2 |
| 11741679_x_at | 1025.91 | 498.75 | 0.49 | SERPINE2 |
| 11726252_a_at | 37.93 | 193.19 | 5.09 | SLC1A3 |
| 11718266_s_at | 742.29 | 354.51 | 0.48 | SMAD3 |
| 11724014_a_at | 189.39 | 770.49 | 4.07 | SOAT1 |
| 11755046_a_at | 89.91 | 481.34 | 5.35 | SPHK1 |
| 11727790_x_at | 31.69 | 65.23 | 2.06 | SPP1 |
| 11720124_at | 165.68 | 353.57 | 2.13 | SRD5A3 |
| 11715959_a_at | 144.81 | 441.89 | 3.05 | SREBF1 |
| 11750740_a_at | 383.24 | 899.13 | 2.35 | ST3GAL5 |
| 11750279_a_at | 99.04 | 1178.52 | 11.90 | STC1 |
| 11746313_a_at | 109.36 | 32.14 | 0.29 | TCF7L2 |
| 11758574_s_at | 465.17 | 210.37 | 0.45 | THRB |
| 11748279_s_at | 40.66 | 134.84 | 3.32 | TM7SF2 |
| 11759179_at | 136.75 | 779.67 | 5.70 | TMEM38B |
| 11727389_a_at | 42.10 | 104.77 | 2.49 | TRPV2 |
| 11725385_at | 465.95 | 181.07 | 0.39 | UGCG |
| 11719752_x_at | 50.34 | 155.88 | 3.10 | VAC14 |
| 11753677_a_at | 121.77 | 41.37 | 0.34 | WNT5A |

Detection limit < 20;
REadj Relative expression adjusted to the detection limit

TABLE 3
Table of the set of genes involved in the metabolism of
cholesterol in NHDF and modulated by compound 6 (2 mg/ml)

| Affy U219 | Control | Compound 6 (2 mg/ml) | | Gene |
|---|---|---|---|---|
| Probe Set ID | REadj1 | REadj2 | Fold change | Symbol |
| 11739503_at | 191.50 | 984.71 | 5.14 | ABCA1 |
| 11720620_s_at | 469.81 | 1051.41 | 2.24 | ACLY |
| 11753244_a_at | 288.86 | 111.61 | 0.39 | BDNF |
| 11757013_x_at | 5005.00 | 2406.25 | 0.48 | CAV1 |
| 11731430_a_at | 154.73 | 316.10 | 2.04 | CYP27A1 |
| 11722564_at | 971.84 | 2431.06 | 2.50 | CYP51A1 /// LRRD1 |
| 11754122_x_at | 1025.64 | 2452.25 | 2.39 | DBI |
| 11743808_a_at | 372.14 | 1520.06 | 4.08 | DHCR7 |
| 11717859_a_at | 406.76 | 1116.25 | 2.74 | EBP |
| 11739910_a_at | 69.22 | 202.15 | 2.92 | ELOVL6 |
| 11743314_a_at | 143.48 | 371.52 | 2.59 | FASN |
| 11758249_s_at | 1568.01 | 3321.26 | 2.12 | FDPS |
| 11752486_a_at | 70.66 | 148.29 | 2.10 | G6PD |
| 11750975_x_at | 107.23 | 352.51 | 3.29 | GBA |
| 11727375_a_at | 323.31 | 896.61 | 2.77 | HMGCR |
| 11716987_a_at | 536.29 | 1779.25 | 3.32 | HMGCS1 |
| 11732374_x_at | 68.38 | 225.29 | 3.29 | HSD17B7 |
| 11741502_a_at | 37.45 | 86.47 | 2.31 | HSD3B7 |
| 11739383_a_at | 1124.01 | 2415.02 | 2.15 | IDI1 |
| 11716339_a_at | 450.00 | 1800.90 | 4.00 | INSIG1 |
| 11746974_a_at | 150.81 | 452.83 | 3.00 | LDLR |
| 11738813_a_at | 81.09 | 242.77 | 2.99 | LEPR |
| 11731324_a_at | 127.93 | 359.16 | 2.81 | LSS |
| 11729695_a_at | 191.37 | 658.20 | 3.44 | MVD |
| 11743916_a_at | 63.03 | 483.98 | 7.68 | NPC1 |
| 11720522_a_at | 252.77 | 518.82 | 2.05 | NSDHL |
| 11723499_a_at | 43.13 | 151.13 | 3.50 | PCSK9 |
| 11716114_x_at | 112.82 | 248.72 | 2.20 | POR |
| 11757184_a_at | 260.48 | 611.01 | 2.35 | SC5D |
| 11724014_a_at | 189.39 | 770.49 | 4.07 | SOAT1 |
| 11727790_x_at | 31.69 | 65.23 | 2.06 | SPP1 |
| 11715959_a_at | 144.81 | 441.89 | 3.05 | SREBF1 |
| 11748279_s_at | 40.66 | 134.84 | 3.32 | TM7SF2 |
| 11731896_a_at | 269.10 | 83.07 | 0.31 | TNFSF4 |
| 11753677_a_at | 121.77 | 41.37 | 0.34 | WNT5A |

Detection limit < 20;
REadj Relative expression adjusted to the detection limit

TABLE 4
Table of the set of genes involved in the synthesis of cholesterol
in NHDF and modulated by compound 6 (2 mg/ml)

| Affy U219 | Control | Compound 6 (2 mg/ml) | | Gene |
|---|---|---|---|---|
| Probe Set ID | REadj1 | REadj2 | Fold change | Symbol |
| 11720620_s_at | 469.81 | 1051.41 | 2.24 | ACLY |
| 11735902_a_at | 60.36 | 27.28 | 0.45 | BDNF |
| 11753244_a_at | 288.86 | 111.61 | 0.39 | BDNF |
| 11757013_x_at | 5005.00 | 2406.25 | 0.48 | CAV1 |
| 11731430_a_at | 154.73 | 316.10 | 2.04 | CYP27A1 |
| 11722564_at | 971.84 | 2431.06 | 2.50 | CYP51A1 /// LRRD1 |
| 11754122_x_at | 1025.64 | 2452.25 | 2.39 | DBI |
| 11743808_a_at | 372.14 | 1520.06 | 4.08 | DHCR7 |
| 11717859_a_at | 406.76 | 1116.25 | 2.74 | EBP |
| 11739910_a_at | 69.22 | 202.15 | 2.92 | ELOVL6 |
| 11743314_a_at | 143.48 | 371.52 | 2.59 | FASN |
| 11758249_s_at | 1568.01 | 3321.26 | 2.12 | FDPS |
| 11752486_a_at | 70.66 | 148.29 | 2.10 | G6PD |
| 11727375_a_at | 323.31 | 896.61 | 2.77 | HMGCR |
| 11716987_a_at | 536.29 | 1779.25 | 3.32 | HMGCS1 |
| 11732374_x_at | 68.38 | 225.29 | 3.29 | HSD17B7 |
| 11744474_s_at | 978.39 | 2141.79 | 2.19 | IDI1 |
| 11716339_a_at | 450.00 | 1800.90 | 4.00 | INSIG1 |
| 11746974_a_at | 150.81 | 452.83 | 3.00 | LDLR |
| 11731324_a_at | 127.93 | 359.16 | 2.81 | LSS |
| 11729695_a_at | 191.37 | 658.20 | 3.44 | MVD |
| 11743916_a_at | 63.03 | 483.98 | 7.68 | NPC1 |
| 11720522_a_at | 252.77 | 518.82 | 2.05 | NSDHL |
| 11716114_x_at | 112.82 | 248.72 | 2.20 | POR |
| 11757184_a_at | 260.48 | 611.01 | 2.35 | SC5D |
| 11715563_s_at | 116.60 | 1237.96 | 10.62 | SCD |
| 11724014_a_at | 189.39 | 770.49 | 4.07 | SOAT1 |
| 11727790_x_at | 31.69 | 65.23 | 2.06 | SPP1 |
| 11715959_a_at | 144.81 | 441.89 | 3.05 | SREBF1 |
| 11748279_s_at | 40.66 | 134.84 | 3.32 | TM7SF2 |
| 11753677_a_at | 121.77 | 41.37 | 0.34 | WNT5A |

Detection limit < 20;
REadj Relative expression adjusted to the detection limit

TABLE 5
Table of the set of genes involved in the differentiation
of adipocytes in NHDF and modulated by compound 6 (2 mg/ml)

| Affy U219 | Control | Compound 6 (2 mg/ml) | | Gene |
|---|---|---|---|---|
| Probe Set ID | REadj1 | REadj2 | Fold change | Symbol |
| 11717305_a_at | 1293.06 | 572.21 | 0.44 | ADIRF |
| 11755151_a_at | 239.40 | 116.31 | 0.49 | ARNTL |
| 11743498_at | 21.50 | 61.65 | 2.87 | BMP2 |
| 11751330_a_at | 483.82 | 1344.33 | 2.78 | CCND1 |
| 11743077_s_at | 859.60 | 2004.45 | 2.33 | CEBPB |
| 11722970_a_at | 190.33 | 21.63 | 0.11 | CREB5 |
| 11726800_at | 344.87 | 98.61 | 0.29 | EBF1 |
| 11722728_a_at | 990.11 | 288.75 | 0.29 | EGR2 |
| 11742981_a_at | 191.71 | 3032.89 | 15.82 | FABP3 |
| 11750247_x_at | 109.90 | 51.42 | 0.47 | GPER1 |
| 11729227_a_at | 688.27 | 309.23 | 0.45 | GRK5 |
| 11728076_at | 34.29 | 229.69 | 6.70 | HDAC9 |
| 11740656_a_at | 269.41 | 555.33 | 2.06 | HMGA2 |
| 11753445_a_at | 46.17 | 793.59 | 17.19 | HMOX1 |
| 11746878_s_at | 1682.91 | 351.21 | 0.21 | ID2 |
| 11746463_a_at | 35.02 | 71.08 | 2.03 | IL6 |
| 11716339_a_at | 450.00 | 1800.90 | 4.00 | INSIG1 |
| 11719634_a_at | 466.86 | 173.54 | 0.37 | KLF4 |
| 11722282_a_at | 533.59 | 219.35 | 0.41 | LAMA4 |
| 11750566_a_at | 132.38 | 393.27 | 2.97 | LPIN1 |
| 11736361_at | 85.68 | 36.64 | 0.43 | MEDAG |
| 11741897_a_at | 1776.01 | 7520.91 | 4.23 | MMP1 |
| 11721124_s_at | 168.43 | 1496.92 | 8.89 | MMP11 |
| 11720852_s_at | 93.33 | 33.92 | 0.36 | NFIA |
| 11717994_a_at | 64.72 | 146.23 | 2.26 | NR4A1 |
| 11729058_s_at | 21.62 | 158.35 | 7.33 | NR4A3 |
| 11742478_a_at | 123.54 | 40.79 | 0.33 | NRG1 |
| 11718645_a_at | 315.76 | 726.40 | 2.30 | OSBPL8 |
| 11758315_s_at | 130.66 | 51.86 | 0.40 | PER2 |
| 11743062_a_at | 73.52 | 201.59 | 2.74 | PLAUR |
| 11730106_a_at | 358.00 | 145.11 | 0.41 | PLCB1 |
| 11756587_a_at | 431.73 | 2172.46 | 5.03 | PTGDS |
| 11715757_a_at | 1808.94 | 4014.23 | 2.22 | RGS2 |
| 11725675_a_at | 103.15 | 36.42 | 0.35 | RORA |
| 11736796_a_at | 281.49 | 92.83 | 0.33 | RUNX1T1 |
| 11715563_s_at | 116.60 | 1237.96 | 10.62 | SCD |
| 11730390_at | 454.02 | 223.22 | 0.49 | SEMA3A |
| 11720606_a_at | 114.37 | 23.60 | 0.21 | SFRP2 |
| 11723123_at | 337.32 | 891.78 | 2.64 | SH3PXD2B |
| 11718266_s_at | 742.29 | 354.51 | 0.48 | SMAD3 |
| 11725514_a_at | 130.40 | 352.04 | 2.70 | SMAD7 |
| 11727790_x_at | 31.69 | 65.23 | 2.06 | SPP1 |
| 11715959_a_at | 144.81 | 441.89 | 3.05 | SREBF1 |
| 11746313_a_at | 109.36 | 32.14 | 0.29 | TCF7L2 |
| 11715437_at | 5084.88 | 1753.12 | 0.34 | TIMP3 |
| 11758074_s_at | 759.07 | 162.68 | 0.21 | VGLL3 |
| 11753677_a_at | 121.77 | 41.37 | 0.34 | WNT5A |

Detection limit < 20;
REadj Relative expression adjusted to the detection limit

TABLE 6
Table of the set of genes involved in the fibrogenesis
in NHDF and modulated by compound 6 (2 mg/ml)

| Affy U219 | Control | Compound 6 (2 mg/ml) | | Gene |
|---|---|---|---|---|
| Probe Set ID | REadj1 | REadj2 | Symbol | Symbol |
| 11718943_a_at | 453.51 | 64.74 | 0.14 | AURKA |
| 11740133_a_at | 1379.98 | 674.91 | 0.49 | CALD1 |
| 11757013_x_at | 5005.00 | 2406.25 | 0.48 | CAV1 |
| 11729334_a_at | 942.37 | 427.85 | 0.45 | CAV2 |
| 11716384_at | 381.29 | 89.22 | 0.23 | CCL2 |
| 11751330_a_at | 483.82 | 1344.33 | 2.78 | CCND1 |
| 11715915_a_at | 2481.86 | 1170.66 | 0.47 | CD44 |
| 11750512_x_at | 578.14 | 1235.99 | 2.14 | CDK4 |
| 11758253_s_at | 433.99 | 198.39 | 0.46 | CDK5RAP2 |
| 11723244_at | 381.06 | 148.44 | 0.39 | CDK6 |
| 11717272_at | 1510.78 | 478.15 | 0.32 | COL5A1 |
| 11755080_a_at | 259.89 | 74.39 | 0.29 | DAAM1 |
| 11728054_a_at | 83.37 | 37.73 | 0.45 | DIAPH3 |
| 11725713_a_at | 101.81 | 20.00 | 0.20 | DIRAS3 |
| 11719488_at | 47.40 | 275.41 | 5.81 | EDNRA |
| 11754442_x_at | 513.27 | 62.57 | 0.12 | ELN |
| 11715412_a_at | 45.89 | 105.05 | 2.29 | EPAS1 |
| 11720508_at | 245.58 | 84.15 | 0.34 | FBN1 |
| 11748655_x_at | 955.81 | 262.30 | 0.27 | FHL1 |
| 11750623_a_at | 376.19 | 164.33 | 0.44 | FILIP1L |
| 11741000_at | 134.03 | 34.69 | 0.26 | GAP43 |
| 11725364_x_at | 483.96 | 150.15 | 0.31 | GAS7 |
| 11723239_a_at | 31.49 | 64.61 | 2.05 | GNG7 |
| 11756874_a_at | 103.55 | 20.00 | 0.19 | GRP |
| 11725931_at | 165.83 | 68.73 | 0.41 | HSPA5 |
| 11728104_at | 23.75 | 97.37 | 4.10 | HTR2B |
| 11746463_a_at | 35.02 | 71.08 | 2.03 | IL6 |
| 11758208_s_at | 242.31 | 114.70 | 0.47 | KLF2 |
| 11731500_a_at | 161.01 | 54.68 | 0.34 | KRT19 |
| 11739505_a_at | 855.14 | 146.20 | 0.17 | LMCD1 |
| 11732567_at | 82.50 | 247.18 | 3.00 | MBP |
| 11723215_s_at | 57.42 | 319.31 | 5.56 | MEF2C |
| 11755860_a_at | 102.16 | 867.65 | 8.49 | MME |
| 11745767_a_at | 835.50 | 1712.63 | 2.05 | MMP2 |
| 11718541_a_at | 20.00 | 254.80 | 12.74 | MTSS1 |
| 11751351_x_at | 144.98 | 494.76 | 3.41 | MYADM |
| 11757621_a_at | 494.87 | 194.10 | 0.39 | MYLK |
| 11717994_a_at | 64.72 | 146.23 | 2.26 | NR4A1 |
| 11742820_s_at | 77.18 | 20.00 | 0.26 | OGN |
| 11755122_a_at | 232.07 | 539.72 | 2.33 | PALLD |
| 11737039_a_at | 30.88 | 116.60 | 3.78 | PHLDB2 |
| 11732188_at | 278.15 | 558.97 | 2.01 | PI4K2A |
| 11743062_a_at | 73.52 | 201.59 | 2.74 | PLAUR |
| 11749527_a_at | 2918.20 | 625.75 | 0.21 | POSTN |
| 11756587_a_at | 431.73 | 2172.46 | 5.03 | PTGDS |
| 11725793_s_at | 150.61 | 63.11 | 0.42 | PTGER4 |
| 11753427_a_at | 788.51 | 1616.27 | 2.05 | RND3 |
| 11725495_a_at | 73.71 | 35.43 | 0.48 | ROBO1 |
| 11743112_at | 2837.01 | 1246.99 | 0.44 | S100A10 |
| 11723123_at | 337.32 | 891.78 | 2.64 | SH3PXD2B |
| 11721399_a_at | 1630.16 | 667.81 | 0.41 | SLIT2 |
| 11718266_s_at | 742.29 | 354.51 | 0.48 | SMAD3 |
| 11755046_a_at | 89.91 | 481.34 | 5.35 | SPHK1 |
| 11753988_a_at | 323.42 | 790.84 | 2.45 | SPRY2 |
| 11759880_at | 89.91 | 41.43 | 0.46 | STARD13 |
| 11717301_at | 98.57 | 20.00 | 0.20 | TACSTD2 |
| 11744469_a_at | 106.46 | 218.25 | 2.05 | TBCD |
| 11718900_a_at | 2084.69 | 764.31 | 0.37 | TGFBR3 |
| 11715542_s_at | 2035.90 | 713.73 | 0.35 | THY1 |
| 11752009_a_at | 1551.35 | 539.50 | 0.35 | TNC |

Detection limit < 20;
REadj Relative expression adjusted to the detection limit

TABLE 7
Table of the set of genes involved in the tensile strength
of skin in NHDF and modulated by compound 6 (2 mg/ml)

| Affy U219 | Control | Compound 6 (2 mg/ml) | | Gene |
|---|---|---|---|---|
| Probe Set ID | REadj1 | REadj2 | Fold change | Symbol |
| 11717272_at | 1510.78 | 478.15 | 0.32 | COL5A1 |
| 11739136_at | 101.95 | 34.47 | 0.34 | COL5A2 |
| 11763262_at | 367.37 | 92.37 | 0.25 | DCN |
| 11744168_at | 2440.02 | 822.90 | 0.34 | DPT |
| 11720508_at | 245.58 | 84.15 | 0.34 | FBN1 |
| 11746597_a_at | 1676.35 | 534.68 | 0.32 | LOX |
| 11736191_s_at | 158.67 | 20.00 | 0.13 | OGN |

Detection limit < 20;
REadj Relative expression adjusted to the detection limit

TABLE 8
Table of the set of genes involved in the synthesis of reactive oxygen
species (ROS) in NHDF and modulated by compound 6 (2 mg/ml)

| Affy U219 | Control | Compound 6 (2 mg/ml) | | |
|---|---|---|---|---|
| Probe Set ID | REadj1 | REadj2 | Fold change | Gene Symbol |
| 11739503_at | 191.50 | 984.71 | 5.14 | ABCA1 |
| 11725176_s_at | 160.10 | 45.20 | 0.28 | AGTR1 |
| 11727478_a_at | 93.80 | 217.80 | 2.32 | AGTRAP |
| 11751921_s_at | 929.38 | 398.39 | 0.43 | AHR |
| 11726693_s_at | 523.22 | 75.26 | 0.14 | ANGPT1 |
| 11732244_at | 127.28 | 62.73 | 0.49 | APOL6 |
| 11743498_at | 21.50 | 61.65 | 2.87 | BMP2 |
| 11757013_x_at | 5005.00 | 2406.25 | 0.48 | CAV1 |
| 11715915_a_at | 2481.86 | 1170.66 | 0.47 | CD44 |
| 11743847_x_at | 479.14 | 143.17 | 0.30 | CFH |
| 11743968_a_at | 951.13 | 374.99 | 0.39 | CYBA |
| 11754122_x_at | 1025.64 | 2452.25 | 2.39 | DBI |
| 11763262_at | 367.37 | 92.37 | 0.25 | DCN |
| 11715412_a_at | 45.89 | 105.05 | 2.29 | EPAS1 |
| 11734659_a_at | 114.05 | 54.42 | 0.48 | FOS |
| 11752577_at | 1624.17 | 3717.86 | 2.29 | FTH1 |
| 11752486_a_at | 70.66 | 148.29 | 2.10 | G6PD |
| 11717036_a_at | 146.22 | 328.14 | 2.24 | HDAC5 |
| 11756138_a_at | 34.64 | 83.57 | 2.41 | HK2 |
| 11753445_a_at | 46.17 | 793.59 | 17.19 | HMOX1 |
| 11731834_a_at | 21.95 | 53.12 | 2.42 | IL24 |
| 11746463_a_at | 35.02 | 71.08 | 2.03 | IL6 |
| 11734006_a_at | 122.52 | 402.06 | 3.28 | IRAK1 |
| 11719634_a_at | 466.86 | 173.54 | 0.37 | KLF4 |
| 11738813_a_at | 81.09 | 242.77 | 2.99 | LEPR |
| 11745775_a_at | 294.06 | 610.89 | 2.08 | LIPA |
| 11740357_a_at | 36.98 | 170.96 | 4.62 | MLPH |
| 11741897_a_at | 1776.01 | 7520.91 | 4.23 | MMP1 |
| 11725987_a_at | 173.40 | 1163.49 | 6.71 | MMP14 |
| 11727361_a_at | 1271.04 | 616.06 | 0.48 | MYLK |
| 11743110_at | 245.44 | 599.69 | 2.44 | NAMPT |
| 11729937_at | 371.06 | 59.78 | 0.16 | NGF |
| 11743916_a_at | 63.03 | 483.98 | 7.68 | NPC1 |
| 11717994_a_at | 64.72 | 146.23 | 2.26 | NR4A1 |
| 11722015_at | 45.39 | 21.38 | 0.47 | OLR1 |
| 11743062_a_at | 73.52 | 201.59 | 2.74 | PLAUR |
| 11742998_at | 2270.96 | 1119.23 | 0.49 | PRDX6 |
| 11749254_a_at | 721.54 | 116.26 | 0.16 | PRSS23 |
| 11753427_a_at | 788.51 | 1616.27 | 2.05 | RND3 |
| 11725675_a_at | 103.15 | 36.42 | 0.35 | RORA |
| 11725905_a_at | 1072.97 | 216.19 | 0.20 | S1PR3 |
| 11763704_a_at | 116.34 | 249.97 | 2.15 | SAT1 |
| 11735152_a_at | 87.34 | 42.56 | 0.49 | SLC8A1 |
| 11718266_s_at | 742.29 | 354.51 | 0.48 | SMAD3 |
| 11725024_a_at | 149.01 | 588.77 | 3.95 | SOD2 |
| 11727790_x_at | 31.69 | 65.23 | 2.06 | SPP1 |
| 11753988_a_at | 323.42 | 790.84 | 2.45 | SPRY2 |
| 11727031_a_at | 120.38 | 706.65 | 5.87 | SQSTM1 |
| 11715959_a_at | 144.81 | 441.89 | 3.05 | SREBF1 |
| 11750279_a_at | 99.04 | 1178.52 | 11.90 | STC1 |
| 11749922_x_at | 1892.09 | 285.39 | 0.15 | TAGLN |
| 11715477_at | 478.12 | 1010.51 | 2.11 | TFRC |
| 11718399_s_at | 37.31 | 228.32 | 6.12 | TGM2 |
| 11730319_at | 1883.51 | 817.04 | 0.43 | TNFRSF11B |
| 11748543_a_at | 335.64 | 94.70 | 0.28 | TXNIP |
| 11750416_a_at | 668.08 | 1574.99 | 2.36 | TXNRD1 |
| 11731558_a_at | 85.58 | 20.20 | 0.24 | WNT2 |
| 11753677_a_at | 121.77 | 41.37 | 0.34 | WNT5A |

Detection limit <20;
REadj Relative expression adjusted to the detection limit

TABLE 9
Table of the set of genes involved in the inflammatory
response in NHDF and modulated by compound 6 (2 mg/ml)

| Affy U219 | Control | Compound 6 (2 mg/ml) | | |
|---|---|---|---|---|
| Probe Set ID | REadj1 | REadj2 | Fold change | Gene Symbol |
| 11722598_s_at | 133.70 | 54.86 | 0.41 | ACKR3 |
| 11725176_s_at | 160.10 | 45.20 | 0.28 | AGTR1 |
| 11739681_x_at | 1366.47 | 648.38 | 0.47 | AHR |
| 11751921_s_at | 929.38 | 398.39 | 0.43 | AHR |
| 11726692_at | 576.71 | 50.51 | 0.09 | ANGPT1 |
| 11718267_a_at | 242.28 | 503.54 | 2.08 | ANGPTL2 |
| 11723974_a_at | 69.71 | 26.73 | 0.38 | APOL3 |
| 11753244_a_at | 288.86 | 111.61 | 0.39 | BDNF |
| 11743498_at | 21.50 | 61.65 | 2.87 | BMP2 |
| 11729846_a_at | 279.83 | 108.39 | 0.39 | BST1 |
| 11757013_x_at | 5005.00 | 2406.25 | 0.48 | CAV1 |
| 11716384_at | 381.29 | 89.22 | 0.23 | CCL2 |
| 11721257_at | 757.36 | 1642.12 | 2.17 | CCND1 |
| 11749694_a_at | 108.30 | 420.66 | 3.88 | CD276 |
| 11715915_a_at | 2481.86 | 1170.66 | 0.47 | CD44 |
| 11763855_x_at | 216.32 | 1014.00 | 4.69 | CD9 |
| 11743077_s_at | 859.60 | 2004.45 | 2.33 | CEBPB |
| 11743847_x_at | 479.14 | 143.17 | 0.30 | CFH |
| 11756316_a_at | 20.00 | 66.35 | 3.32 | CHI3L1 |
| 11760623_at | 64.31 | 30.83 | 0.48 | CKLF /// |
| | | | | CMTM1 |
| 11719182_a_at | 352.34 | 807.64 | 2.29 | CLCN7 |
| 11743968_a_at | 951.13 | 374.99 | 0.39 | CYBA |
| 11715729_s_at | 20.00 | 51.79 | 2.59 | CYP19A1 |
| 11730353_a_at | 323.19 | 119.87 | 0.37 | CYP26B1 |
| 11719488_at | 47.40 | 275.41 | 5.81 | EDNRA |
| 11752940_a_at | 762.43 | 300.02 | 0.39 | EGR1 |
| 11754442_x_at | 513.27 | 62.57 | 0.12 | ELN |
| 11715412_a_at | 45.89 | 105.05 | 2.29 | EPAS1 |
| 11739518_a_at | 271.49 | 66.48 | 0.24 | FLRT3 |
| 11734659_a_at | 114.05 | 54.42 | 0.48 | FOS |
| 11736581_a_at | 47.52 | 20.17 | 0.42 | GCNT1 |
| 11750247_x_at | 109.90 | 51.42 | 0.47 | GPER1 |
| 11724424_at | 254.32 | 733.62 | 2.88 | GPR68 |
| 11717065_a_at | 108.72 | 564.60 | 5.19 | GREM1 |
| 11756874_a_at | 103.55 | 20.00 | 0.19 | GRP |
| 11717036_a_at | 146.22 | 328.14 | 2.24 | HDAC5 |
| 11728076_at | 34.29 | 229.69 | 6.70 | HDAC9 |
| 11753445_a_at | 46.17 | 793.59 | 17.19 | HMOX1 |
| 11757600_x_at | 1145.98 | 467.53 | 0.41 | HSPG2 |
| 11740881_x_at | 143.71 | 49.92 | 0.35 | IL15 |
| 11731834_a_at | 21.95 | 53.12 | 2.42 | IL24 |
| 11746463_a_at | 35.02 | 71.08 | 2.03 | IL6 |
| 11734006_a_at | 122.52 | 402.06 | 3.28 | IRAK1 |
| 11743617_at | 220.00 | 692.91 | 3.15 | ITGA2 |
| 11719634_a_at | 466.86 | 173.54 | 0.37 | KLF4 |
| 11746974_a_at | 150.81 | 452.83 | 3.00 | LDLR |
| 11745775_a_at | 294.06 | 610.89 | 2.08 | LIPA |
| 11718833_a_at | 309.13 | 83.40 | 0.27 | LOXL2 |
| 11722982_a_at | 190.59 | 518.33 | 2.72 | LYST |
| 11733088_at | 190.04 | 73.12 | 0.38 | MAF |
| 11715484_a_at | 175.72 | 412.36 | 2.35 | MCL1 |
| 11750244_a_at | 257.24 | 106.64 | 0.41 | MGLL |
| 11741897_a_at | 1776.01 | 7520.91 | 4.23 | MMP1 |
| 11725987_a_at | 173.40 | 1163.49 | 6.71 | MMP14 |
| 11751287_a_at | 123.78 | 48.75 | 0.39 | MMP19 |
| 11745767_a_at | 835.50 | 1712.63 | 2.05 | MMP2 |
| 11721837_a_at | 848.68 | 351.45 | 0.41 | MYLK |
| 11743010_at | 95.38 | 384.87 | 4.04 | NFIL3 |
| 11729937_at | 371.06 | 59.78 | 0.16 | NGF |
| 11743916_a_at | 63.03 | 483.98 | 7.68 | NPC1 |
| 11717994_a_at | 64.72 | 146.23 | 2.26 | NR4A1 |
| 11742478_a_at | 123.54 | 40.79 | 0.33 | NRG1 |
| 11722015_at | 45.39 | 21.38 | 0.47 | OLR1 |
| 11719880_at | 68.46 | 202.56 | 2.96 | OSTM1 |
| 11720425_a_at | 520.68 | 102.11 | 0.20 | PENK |
| 11743062_a_at | 73.52 | 201.59 | 2.74 | PLAUR |
| 11742107_a_at | 129.02 | 377.32 | 2.92 | PPT1 |
| 11726218_a_at | 210.77 | 630.51 | 2.99 | PRDM1 |
| 11732550_at | 152.04 | 59.34 | 0.39 | PTGER2 |
| 11725793_s_at | 150.61 | 63.11 | 0.42 | PTGER4 |
| 11724441_x_at | 604.04 | 108.89 | 0.18 | PTGIS |
| 11718157_s_at | 4499.29 | 1245.04 | 0.28 | PTX3 |
| 11758934_x_at | 110.92 | 240.19 | 2.17 | RAB27A |
| 11758403_s_at | 525.46 | 187.51 | 0.36 | RAPH1 |
| 11725675_a_at | 103.15 | 36.42 | 0.35 | RORA |
| 11725905_a_at | 1072.97 | 216.19 | 0.20 | S1PR3 |
| 11720030_a_at | 20.00 | 146.85 | 7.34 | SCG2 |
| 11721399_a_at | 1630.16 | 667.81 | 0.41 | SLIT2 |
| 11718266_s_at | 742.29 | 354.51 | 0.48 | SMAD3 |
| 11725514_a_at | 130.40 | 352.04 | 2.70 | SMAD7 |
| 11755046_a_at | 89.91 | 481.34 | 5.35 | SPHK1 |
| 11716526_x_at | 471.25 | 1366.41 | 2.90 | SPON2 |
| 11727790_x_at | 31.69 | 65.23 | 2.06 | SPP1 |
| 11742423_a_at | 187.09 | 499.29 | 2.67 | STEAP2 |
| 11718399_s_at | 37.31 | 228.32 | 6.12 | TGM2 |
| 11721212_a_at | 211.35 | 57.58 | 0.27 | THBS4 |
| 11752009_a_at | 1551.35 | 539.50 | 0.35 | TNC |
| 11745772_x_at | 92.27 | 237.60 | 2.57 | TNFRSF14 |
| 11759254_at | 128.49 | 31.44 | 0.24 | TNFSF18 |
| 11731896_a_at | 269.10 | 83.07 | 0.31 | TNFSF4 |
| 11727389_a_at | 42.10 | 104.77 | 2.49 | TRPV2 |
| 11728388_at | 49.88 | 20.00 | 0.40 | TSPAN2 |
| 11732219_a_at | 455.17 | 186.52 | 0.41 | TUBE1 |
| 11733252_a_at | 1215.55 | 379.41 | 0.31 | UACA |
| 11718569_a_at | 444.07 | 161.21 | 0.36 | WIPF1 |
| 11753677_a_at | 121.77 | 41.37 | 0.34 | WNT5A |

Detection limit <20;
REadj Relative expression adjusted to the detection limit

TABLE 10
Table of the set of genes involved in the chronic inflammatory
disorder in NHDF and modulated by compound 6 (2 mg/ml)

| Affy U219 | Control | Compound 6 (2 mg/ml) | | |
|---|---|---|---|---|
| Probe Set ID | REadj1 | REadj2 | Fold change | Gene Symbol |
| 11739503_at | 191.50 | 984.71 | 5.14 | ABCA1 |
| 11720620_s_at | 469.81 | 1051.41 | 2.24 | ACLY |
| 11727163_a_at | 330.33 | 97.16 | 0.29 | ACVR2A |
| 11725191_s_at | 67.69 | 20.24 | 0.30 | ADGRG6 |
| 11725176_s_at | 160.10 | 45.20 | 0.28 | AGTR1 |
| 11751921_s_at | 929.38 | 398.39 | 0.43 | AHR |
| 11723399_at | 1399.68 | 88.24 | 0.06 | ALPK2 |
| 11726271_a_at | 157.68 | 25.46 | 0.16 | ATP1B1 |
| 11753244_a_at | 288.86 | 111.61 | 0.39 | BDNF |
| 11720531_at | 20.26 | 48.41 | 2.39 | C9orf72 |
| 11754856_a_at | 95.06 | 308.12 | 3.24 | CA12 |
| 11740133_a_at | 1379.98 | 674.91 | 0.49 | CALD1 |
| 11716384_at | 381.29 | 89.22 | 0.23 | CCL2 |
| 11749694_a_at | 108.30 | 420.66 | 3.88 | CD276 |
| 11715915_a_at | 2481.86 | 1170.66 | 0.47 | CD44 |
| 11723244_at | 381.06 | 148.44 | 0.39 | CDK6 |
| 11743077_s_at | 859.60 | 2004.45 | 2.33 | CEBPB |
| 11733262_a_at | 60.98 | 157.45 | 2.58 | CELF2 |
| 11752011_a_at | 33.89 | 91.14 | 2.69 | CEMIP |
| 11744331_a_at | 135.21 | 63.97 | 0.47 | CFB |
| 11743847_x_at | 479.14 | 143.17 | 0.30 | CFH |
| 11756316_a_at | 20.00 | 66.35 | 3.32 | CHI3L1 |
| 11753004_a_at | 156.29 | 68.54 | 0.44 | CKB |
| 11715414_x_at | 2655.33 | 994.03 | 0.37 | COL3A1 |
| 11715524_a_at | 509.77 | 245.44 | 0.48 | COTL1 |
| 11739100_a_at | 348.46 | 159.95 | 0.46 | CSTF1 |
| 11731465_a_at | 353.40 | 75.93 | 0.21 | CTSC |
| 11757307_s_at | 608.37 | 1340.07 | 2.20 | CTSD |
| 11743968_a_at | 951.13 | 374.99 | 0.39 | CYBA |
| 11715729_s_at | 20.00 | 51.79 | 2.59 | CYP19A1 |
| 11722564_at | 971.84 | 2431.06 | 2.50 | CYP51A1 /// |
| | | | | LRRD1 |
| 11715412_a_at | 45.89 | 105.05 | 2.29 | EPAS1 |
| 11740066_a_at | 65.88 | 28.44 | 0.43 | ERCC6 |
| 11758249_s_at | 1568.01 | 3321.26 | 2.12 | FDPS |
| 11748850_a_at | 110.39 | 54.68 | 0.50 | FLT3LG |
| 11734659_a_at | 114.05 | 54.42 | 0.48 | FOS |
| 11726384_at | 92.70 | 45.11 | 0.49 | FRY |
| 11752577_at | 1624.17 | 3717.86 | 2.29 | FTH1 |
| 11758064_s_at | 106.04 | 261.20 | 2.46 | FZD8 |
| 11752486_a_at | 70.66 | 148.29 | 2.10 | G6PD |
| 11726329_x_at | 82.28 | 34.94 | 0.42 | GBP1 |
| 11750247_x_at | 109.90 | 51.42 | 0.47 | GPER1 |
| 11729227_a_at | 688.27 | 309.23 | 0.45 | GRK5 |
| 11756874_a_at | 103.55 | 20.00 | 0.19 | GRP |
| 11727375_a_at | 323.31 | 896.61 | 2.77 | HMGCR |
| 11716939_a_at | 114.80 | 1648.34 | 14.36 | HMOX1 |
| 11753445_a_at | 46.17 | 793.59 | 17.19 | HMOX1 |
| 11754606_a_at | 1580.45 | 688.74 | 0.44 | HSP90B1 |
| 11725931_at | 165.83 | 68.73 | 0.41 | HSPA5 |
| 11715776_a_at | 59.92 | 246.23 | 4.11 | HSPB8 |
| 11728104_at | 23.75 | 97.37 | 4.10 | HTR2B |
| 11728654_x_at | 197.24 | 51.91 | 0.26 | IGFBP5 |
| 11757033_a_at | 41.33 | 121.36 | 2.94 | IL13RA2 |
| 11740881_x_at | 143.71 | 49.92 | 0.35 | IL15 |
| 11731834_a_at | 21.95 | 53.12 | 2.42 | IL24 |
| 11746463_a_at | 35.02 | 71.08 | 2.03 | IL6 |
| 11722503_at | 428.83 | 151.50 | 0.35 | ITGB8 |
| 11742964_a_at | 49.98 | 184.24 | 3.69 | ITPR3 |
| 11729231_at | 51.07 | 123.84 | 2.42 | KLF3 |
| 11722353_s_at | 709.21 | 307.70 | 0.43 | LDB2 |
| 11738813_a_at | 81.09 | 242.77 | 2.99 | LEPR |
| 11755546_a_at | 102.35 | 51.05 | 0.50 | LFNG |
| 11744741_at | 75.93 | 25.40 | 0.33 | LINC00312 /// |
| | | | | LMCD1 |
| 11741376_a_at | 42.14 | 107.00 | 2.54 | LRP8 |
| 11721995_a_at | 150.71 | 902.56 | 5.99 | LRRC32 |
| 11721630_at | 64.16 | 347.37 | 5.41 | MAFB |
| 11715484_a_at | 175.72 | 412.36 | 2.35 | MCL1 |
| 11750244_a_at | 257.24 | 106.64 | 0.41 | MGLL |
| 11740357_a_at | 36.98 | 170.96 | 4.62 | MLPH |
| 11755860_a_at | 102.16 | 867.65 | 8.49 | MME |
| 11741897_a_at | 1776.01 | 7520.91 | 4.23 | MMP1 |
| 11725987_a_at | 173.40 | 1163.49 | 6.71 | MMP14 |
| 11745767_a_at | 835.50 | 1712.63 | 2.05 | MMP2 |
| 11743110_at | 245.44 | 599.69 | 2.44 | NAMPT |
| 11744915_a_at | 510.57 | 120.05 | 0.24 | NEGR1 |
| 11729937_at | 371.06 | 59.78 | 0.16 | NGF |
| 11729507_a_at | 121.22 | 47.04 | 0.39 | NPAS3 |
| 11717994_a_at | 64.72 | 146.23 | 2.26 | NR4A1 |
| 11729058_s_at | 21.62 | 158.35 | 7.33 | NR4A3 |
| 11723535_at | 300.99 | 83.80 | 0.28 | PDGFRL |
| 11743440_at | 216.68 | 106.70 | 0.49 | PDIA3 |
| 11756275_a_at | 465.21 | 226.15 | 0.49 | PDLIM1 |
| 11743062_a_at | 73.52 | 201.59 | 2.74 | PLAUR |
| 11716114_x_at | 112.82 | 248.72 | 2.20 | POR |
| 11728880_s_at | 44.17 | 93.34 | 2.11 | PPP3R1 |
| 11726218_a_at | 210.77 | 630.51 | 2.99 | PRDM1 |
| 11718241_a_at | 108.69 | 553.69 | 5.09 | PRUNE2 |
| 11726677_a_at | 205.67 | 100.81 | 0.49 | PSMB9 |
| 11732550_at | 152.04 | 59.34 | 0.39 | PTGER2 |
| 11725793_s_at | 150.61 | 63.11 | 0.42 | PTGER4 |
| 11724441_x_at | 604.04 | 108.89 | 0.18 | PTGIS |
| 11758664_s_at | 898.12 | 307.98 | 0.34 | RHOBTB3 |
| 11736796_a_at | 281.49 | 92.83 | 0.33 | RUNX1T1 |
| 11743113_x_at | 2671.54 | 1129.52 | 0.42 | S100A10 |
| 11724117_x_at | 94.40 | 33.66 | 0.36 | SAMD9L |
| 11734371_a_at | 48.00 | 140.05 | 2.92 | SCML1 |
| 11748053_x_at | 117.28 | 500.42 | 4.27 | SERINC2 |
| 11718492_at | 452.19 | 96.38 | 0.21 | SLC1A2 |
| 11724454_at | 152.03 | 742.00 | 4.88 | SLC22A4 |
| 11725514_a_at | 130.40 | 352.04 | 2.70 | SMAD7 |
| 11719365_x_at | 354.11 | 160.36 | 0.45 | SPATS2L |
| 11755046_a_at | 89.91 | 481.34 | 5.35 | SPHK1 |
| 11727790_x_at | 31.69 | 65.23 | 2.06 | SPP1 |
| 11753988_a_at | 323.42 | 790.84 | 2.45 | SPRY2 |
| 11730551_a_at | 96.44 | 35.64 | 0.37 | STAC |
| 11744469_a_at | 106.46 | 218.25 | 2.05 | TBCD |
| 11746313_a_at | 109.36 | 32.14 | 0.29 | TCF7L2 |
| 11715477_at | 478.12 | 1010.51 | 2.11 | TFRC |
| 11758574_s_at | 465.17 | 210.37 | 0.45 | THRB |
| 11725902_s_at | 63.20 | 136.50 | 2.16 | TMEM135 |
| 11734005_s_at | 20.38 | 47.30 | 2.32 | TMEM192 |
| 11752009_a_at | 1551.35 | 539.50 | 0.35 | TNC |
| 11730319_at | 1883.51 | 817.04 | 0.43 | TNFRSF11B |
| 11731896_a_at | 269.10 | 83.07 | 0.31 | TNFSF4 |
| 11735913_s_at | 563.25 | 236.34 | 0.42 | TNXA /// TNXB |
| 11753677_a_at | 121.77 | 41.37 | 0.34 | WNT5A |

Detection limit <20;
REadj Relative expression adjusted to the detection limit

### Analysis of Signalling Pathway

A more advanced bioinformatics analysis was performed using the Ingenuity Pathway Analysis software (IPA from Qiagen®). This analysis allows the identification of the impacted signalling pathways and predicts their modulation. The modulation is a stimulation when the Activation z-score is a positive value (Table 11) and an inhibition when the Activation z-score is a negative value (Table 12).

TABLE 11
Predictive stimulation of signalling pathway by compound 6 (2 mg/ml) on NHDF

| Diseases or | | | |
|---|---|---|---|
| Functions | | Activation | |
| Annotation | p-Value | z- score | Molecules |
| Synthesis of | 1.22E−21 | 1.380 | ABCA1, ABHD3, ACADVL, ACLY, ACSL3, |
| lipids | | | ACSS2, AGTR1, AHR, AKR1B1, AKR1C1/ |
| | | | AKR1C2, AKR1C3, ALDH1A3, ANGPT1, |
| | | | ARNTL, ASAH1, B4GALT6, BDNF, |
| | | | BHLHE40, BMP2, CAV1, CAV2, CCL2, CD9, |
| | | | CEBPB, CERS1, CHKA, CLN3, COTL1, |
| | | | CYP19A1, CYP27A1, CYP51A1, DAB1, |
| | | | DBI, DHCR7, DLAT, EBF1, EBP, EDNRA, |
| | | | EGR1, ELOVL6, ETV1, FADS1, FADS2, |
| | | | FASN, FDPS, FOS, FOSL1, G6PD, GBA, |
| | | | GDF15, GK, GPER1, GRP, HACD1, HMGCR, |
| | | | HMGCS1, HMOX1, HSD17B14, HSD17B7, |
| | | | HSD3B7, HSPA5, HTR2B, IDI1, IL15, IL24, |
| | | | IL6, INSIG1, KDSR, KLF11, KLF4, LDLR, |
| | | | LEPR, LPIN1, LSS, ME1, MGST2, MMP2, |
| | | | MVD, NFIL3, NGF, NPC1, NPC2, NR4A1, |
| | | | NR4A3, NRG1, NSDHL, NSMAF, OLR1, |
| | | | PCYT2, PDE5A, PDIA3, PFKFB2, PGD, |
| | | | PI4K2A, PITPNM3, PLAAT3, PLCB1, PLPP1, |
| | | | PLPP2, POR, PPT1, PRKAG2, PTGDS, |
| | | | PTGER2, PTGIS, PTX3, RAB27A, RGS2, |
| | | | RGS3, RUNX1, S1PR3, SC5D, SCD, SEMA3A, |
| | | | SERINC2, SERPINE2, SLC1A3, SMAD3, |
| | | | SOAT1, SPHK1, SPP1, SRD5A3, SREBF1, |
| | | | ST3GAL5, STC1, TCF7L2, THRB, TM7SF2, |
| | | | TMEM38B, TRPV2, UGCG, VAC14, WNT5A |
| Metabolism | 6.27E−18 | 1.421 | ABCA1, ACLY, BDNF, CAV1, CYP27A1, |
| of cholesterol | | | CYP51A1, DBI, DHCR7, EBP, ELOVL6, |
| | | | FASN, FDPS, G6PD, GBA, HMGCR, HMGCS1, |
| | | | HSD17B7, HSD3B7, IDI1, INSIG1, LDLR, |
| | | | LEPR, LSS, MVD, NPC1, NSDHL, PCSK9, |
| | | | PIP4P1, POR, SC5D, SCD, SOAT1, SPP1, |
| | | | SREBF1, TM7SF2, TNFSF4, WNT5A |
| Synthesis of | 1.57E−17 | 0.998 | ACLY, BDNF, CAVI, CYP27A1, CYP51A1, |
| cholesterol | | | DBI, DHCR7, EBP, ELOVL6, FASN, FDPS, |
| | | | G6PD, HMGCR, HMGCS1, HSD17B7, IDI1, |
| | | | INSIG1, LDLR, LSS, MVD, NPC1, NSDHL, |
| | | | POR, SC5D, SCD, SOAT1, SPP1, SREBF1, |
| | | | TM7SF2, WNT5A |
| Differentiation | 7.64E−12 | 0.545 | ADIRF, ARNTL, BMP2, CAVIN1, CCND1, |
| of adipocytes | | | CEBPB, CREB5, EBF1, EGR2, FABP3, GPER1, |
| | | | GRK5, HDAC9, HMGA2, HMOX1, ID2, |
| | | | IL6, INSIG1, KLF4, LAMA4, LPIN1, |
| | | | MEDAG, MMP1, MMP11, NFIA, NR4A1, |
| | | | NR4A3, NRG1, OSBPL8, PER2, PLAUR, |
| | | | PLCB1, PTGDS, RGS2, RORA, RUNX1T1, |
| | | | SCD, SEMA3A, SFRP2, SH3PXD2B, SMAD3, |
| | | | SMAD7, SOD2, SPP1, SREBF1, TCF7L2, |
| | | | TIMP3, VGLL3, WNT5A |

The analysis of signalling pathways has shown a predictive activation of the lipid synthesis and the cholesterol biosynthetic process and the adipocytes differentiation at a transcriptional level by compound 6.

Thus, the treatment of NHDF with compound 6 resulted in an up regulation of lipid and cholesterol synthesis, as well as the differentiation of adipocytes.

TABLE 12
Predictive inhibition of signalling pathway by compound 6 (2 mg/ml) on NHDF

| Diseases or | | | |
|---|---|---|---|
| Functions | | Activation | |
| Annotation | p-Value | z- score | Molecules |
| Fibrogenesis | 1.47E−06 | −1.959 | AURKA, CALD1, CAV1, CCL2, CCND1, |
| | | | CD44, CDK4, CDK5RAP2, CDK6, COL5A1, |
| | | | DAAM1, DIAPH3, DIRAS3, EDNRA, ELN, |
| | | | EPAS1, FBN1, FHL1, FILIP1L, GAP43, |
| | | | GAS7, GNG7, GRP, HSPA5, HTR2B, IL6, |
| | | | KLF2, KRT19, LMCD1, LMOD1, MBP, |
| | | | MEF2C, MME, MMP2, MTSS1, MYADM, |
| | | | MYLK, NR4A1, OGN, PALLD, PHLDB2, |
| | | | PI4K2A, PLAUR, POSTN, PTGDS, PTGER4, |
| | | | RND3, ROBO1, S100A10, SH3PXD2B, |
| | | | SLIT2, SMAD3, SPHK1, SPRY2, STARD13, |
| | | | TACSTD2, TBCD, TGFBR3, THY1, TNC |
| Tensile | 2.95E−06 | −2.195 | COL14A1, COL5A1, COL5A2, DCN, DPT, |
| strength of skin | | | FBN1, LOX, OGN |
| Synthesis of | 3.30E−06 | −1.989 | ABCA1, AGTR1, AGTRAP, AHR, AKR1B1, |
| reactive | | | ANGPT1, APOL6, BMP2, CAV1, CD44, |
| oxygen species | | | CFB, CFH, CYBA, DBI, DCN, EPAS1, FOS, |
| | | | FTH1, G6PD, HDAC5, HK2, HMOX1, IL24, |
| | | | IL6, IRAK1, KLF4, LEPR, LIPA, MLPH, |
| | | | MMP1, MMP14, MYLK, NAMPT, NGF, NPC1, |
| | | | NR4A1, OLR1, PLAUR, PRDX6, PRSS23, |
| | | | RND3, RORA, S1PR3, SAT1, SLC8A1, |
| | | | SMAD3, SOD2, SPP1, SPRY2, SQSTM1, |
| | | | SREBF1, STC1, TAGLN, TFRC, TGM2, |
| | | | TNFRSF11B, TXNIP, TXNRD1, WNT2, |
| | | | WNT5A |
| Inflammatory | 1.48E−08 | −0.905 | ACKR3, AGTR1, AHR, ANGPT1, ANGPTL2, |
| response | | | APOL3, BDNF, BMP2, BST1, CAV1, CCL2, |
| | | | CCND1, CD276, CD44, CD9, CEBPB, CFH, |
| | | | CHI3L1, CKLF, CLCN7, CYBA, CYP19A1, |
| | | | CYP26B1, EDNRA, EGR1, ELN, EPAS1, |
| | | | FLT3LG, FOS, GCNT1, GPER1, GPR68, |
| | | | GREM1, GRP, HDAC5, HDAC9, HMOX1, |
| | | | HSPG2, IL15, IL24, IL6, IRAK1, ITGA2, |
| | | | KLF4, LDLR, LIPA, LOXL2, LYST, MAF, |
| | | | MCL1, MGLL, MMP1, MMP14, MMP19, |
| | | | MMP2, MYLK, NFIL3, NGF, NPC1, NR4A1, |
| | | | NRG1, OLR1, OSTM1, PENK, PLAUR, |
| | | | PPT1, PRDM1, PTGER2, PTGER4, PTGIS, |
| | | | PTX3, RAB27A, RAPH1, RORA, S1PR3, |
| | | | SCG2, SLIT2, SMAD3, SMAD7, SPHK1, |
| | | | SPON2, SPP1, STEAP2, TGM2, THBS4, |
| | | | TNC, TNFRSF14, TNFSF18, TNFSF4, |
| | | | TRPV2, TSPAN2, TUBE1, UACA, VSIR, |
| | | | WIPF1, WNT5A |
| Chronic | 2.18E−06 | −0.647 | ABCA1, ACLY, ACVR2A, ADGRG6, AGTR1, |
| inflammatory | | | AHR, ALPK2, ATP1B1, BDNF, C9orf72, |
| disorder | | | CA12, CALD1, CCL2, CD276, CD44, |
| | | | CDK6, CEBPB, CELF2, CEMIP, CFB, CFH, |
| | | | CHI3L1, CKB, COL3A1, COTL1, CSTF1, |
| | | | CTSC, CTSD, CYBA, CYP19A1, CYP51A1, |
| | | | EPAS1, ERCC6, FDPS, FLT3LG, FOS, FRY, |
| | | | FTH1, FZD8, G6PD, GBP1, GPER1, GRK5, |
| | | | GRP, HMGCR, HMOX1, HSP90B1, HSPA5, |
| | | | HSPB8, HTR2B, IGFBP5, IL13RA2, |
| | | | IL15, IL24, IL6, ITGB8, ITPR3, KLF3, LDB2, |
| | | | LEPR, LFNG, LMCD1, LRP8, LRRC32, |
| | | | MAFB, MCL1, MGLL, MLPH, MME, MMP1, |
| | | | MMP14, MMP2, NAMPT, NEGR1, NGF, |
| | | | NPAS3, NR4A1, NR4A3, PDE5A, PDGFRL, |
| | | | PDIA3, PDLIM1, PLAUR, POR, PPP3R1, |
| | | | PRDM1, PRUNE2, PSMB9, PTGER2, PTGER4, |
| | | | PTGIS, RFLNB, RHOBTB3, RUNX1, |
| | | | S100A10, SAMD9L, SCML1, SERINC2, |
| | | | SLC1A2, SLC22A4, SMAD7, SPATS2L, |
| | | | SPHK1, SPP1, SPRY2, STAC, TBCD, TCF7L2, |
| | | | TFRC, THRB, TMEM135, TMEM192, |
| | | | TNC, TNFRSF11B, TNFSF4, TNXB, WNT5A |

The analysis of signaling pathways has shown a predictive inhibition of the fibrogenesis, the tensile strength of skin, the synthesis of ROS (reactive oxygen species), the inflammatory response and chronic inflammatory disorder at a transcriptional level by compound 6.

Thus, the treatment of NHDF with compound 6 resulted in a down regulation of the fibrogenesis, the tensile strength of skin, the synthesis of ROS, as well as the inflammatory response and chronic inflammatory disorder.

We have shown in another experiment that the treatment of aged human fibroblasts with compound 6 at 6 mg/ml resulted in an increased SOD2 gene expression by 204% compared to the control. That showed that the compound is involved in the oxidative and cellular stress response in aged human fibroblasts.

2.2. Effect of Compound 6 on the Preservation/Protection of Neonatal Skin Fibroblasts Under Starvation Conditions. Evaluation by Neutral Red Uptake Assay.

### Materials and Methods

### Subculturing

The neonatal skin fibroblasts (Cell line: CCD-27SK. ATCC number CRL-1475) were grown with DMEM medium supplemented with Fetal Bovine Serum 10% final, antibiotics (Penicillin/Streptomycin) 1% final and Amphotericin B 0.1% final. Fibroblasts were grown in 75 cm² culture flask to 80% confluence in 37° C. and 10% CO₂ incubator. The medium was changed every two days by 37° C. preheated fresh medium.

### Starvation Medium

This medium was composed of 45% subculturing medium without Fetal Bovine Serum mixed with 55% of Phosphate Buffer Saline IX containing EDTA (final concentration of 0.45 mM). This was referred to as serum-free medium or starvation medium.

### Product Preparation

Compound 6 (MM=356.3 g/mol) was diluted in starvation medium to 17 mM final and pH was adjusted at 7.4 by addition of NaOH 1N.

### General Experimental Procedure

### Assays on 96 Well Plates

Fibroblast cells were concentrated to 2.10⁵ cells/ml and 100 μl of cell suspension was added in wells of a 96-well plate and incubated in 37° C. and 10% CO₂ incubator for 4 hours.

After cell adhesion the medium was changed and plates were incubated (37° C.-10% CO₂) to perform the assay as follows:
One plate for each sampling times: D0, D4, D7 days;
Three wells for each condition (triplicate count) added with 120 μl of culture medium (surviving control), starvation medium (serum-free control) or compound 6 solution at 17 mM.

### Viability Assay (Neutral Red Uptake)

The neutral red uptake assay was used for the determination of cell viability. This assay is based on the ability of viable cells to incorporate and bind the supravital dye neutral red in its lysosomes. Thus, only the viable cells are dyed. At D4 and D7, the plates were incubated with neutral red solution for 3.5 hours. The cells are subsequently washed, the dye is extracted in each well and the absorbance is read using a spectrophotometer.

For sampling, 1 mL of DMEM (without phenol red indicator) with neutral red (OD=0.110) was added to the cells for 3.5 hours (37° C. 10% CO₂). After incubation. the medium was removed. Two washes with PBS were realized and 1 mL of extraction solution (absolute ethanol 49%, ultrapure water 49%, glacial acetic acid 2%) was added. Plates were placed 15 minutes on rotary shaker in the dark before reading OD at 540 nm.

The OD540 nm average values were compared and the variation of viability was calculated as follows:

Variation of viability at Dx=(OD540 nm of tested solution−blank) at Dx/(OD540 nm of stress control−blank) at Dx.

### Results

The cell viability (OD 540 nm) from stressed cultures added with tested compounds were compared with stress-control culture at different times and the variation of viability was calculated. The results are presented in the following Table 13.

TABLE 13
Preservative effect of compound 6 at 17 mM on human fibroblasts
culture for 7 days after serum deprivation

| Culture conditions | D 4 | D 7 |
|---|---|---|
| Stress control (starvation medium) | 0.118 | 0.066 |
| (OD 540 nm − mean value) | | |
| Stress + compound 6 at 17 mM | 0.274 | 0.224 |
| (OD 540 nm − mean value) | | |
| Variation of viability | 2.32 | 3.39 |
| (stressed cells treated with compound 6 vs stressed cells) | | |

The viability of cells cultured in starvation medium but treated with compound 6 at 17 mM is 2.3 times higher than that of the cells in the serum-free control after 4 days of culture and 3.4 times higher after 7 days. Thus, compound 6 showed a significant preservative/protective effect on skin fibroblasts since cells have been maintained in a healthy state under unfavorable conditions for growth.

### 2.3. Evaluation of Protective and Pro-Adipogenic, Anti-Inflammatory and Anti-Aging Properties of Compound 6

In order to evaluate and characterize its protective effect and its pro-adipogenic, anti-inflammatory and anti-aging properties, the effect of compound 6 has been tested on human dermal fibroblast and human pre-adipocytes proliferation, either in normal or fibro-inflammatory environment.

### Methods

### Cell Culture

Human dermal fibroblasts were isolated from skin tissue by dermis explants seeding in Petri dishes in DMEM-20% FBS for 3 weeks. Dermal fibroblasts were then seeded in 96-well plates and then incubated in different culture conditions described below.

Different culture conditions were realized in triplicate at least:
Positive control of cytotoxicity: cells in DMEM 1% FBS medium lysed by 0.2% triton at the end of the culture period
Control: cells in DMEM 1% FBS medium
Test: cells in DMEM 1% FBS medium added with test compound 6 and compound 44 of WO2015/140178.

Preadipocytes have been isolated from human female hypodermis (body mass index <30 kg/m2 and <45 years old). Preadipocytes have been cultured for 24 h in 100 μl of DMEM-10% Fetal Bovine Serum (FBS) in 96-well plates. Then cells were treated to induce their differentiation in a classical or an inflammatory environment for 13 days.

To induce the preadipocytes differentiation cells were incubated in a proadipogenic cocktail (PAC) including insulin, glucocorticoid, 3-isobutyl-1-methylxanthine (IBMX), and thiazolinedione in DMEM.

To induce a fibro-inflammatory environment, cells were treated with an activated human macrophage-conditioned medium (AcMC) prepared in RPMI medium. A treatment with Dexamethasone (DXM) at 100 nM was used as anti-inflammatory response control. At D0: preadipocytes have been treated in the following conditions:
“Undifferentiation”: DMEM+¼ RPMI medium
“Differentiation”: PAC+¼ RPMI medium
“AcMC” i.e. inflammatory condition: PAC+¼ AcMC
“AcMC+DXM”: PAC+14 AcMC+anti-inflammatory DXM at 100 nM
“AcMc+compounds”: PAC+14 AcMC+compounds to be tested

All conditions have been performed in triplicate. The medium has been changed every 2 days for 13 days.

At D14: during the last 24 h of culture, the medium has been replaced by DMEM/F12 medium in all conditions, to collect cells' secretions.

### Tested Compounds

The effects of compound 6 and compound 44 of WO2015/140178 have been evaluated at different concentrations: in culture media (DMEM).

### Cell Cytotoxicity

Cytotoxicity was assessed by the measurement of the lactate dehydrogenase (LDH) released by damaged cells in the culture medium (using the kit CytoTox-One Non-Radioactive Cytotoxicity Assay, G1780, Promega). Cells were treated with 0.2% of triton at the end of the culture to determine the maximal toxicity response. The LDH measurement was realized on 24 h medium secretions after 13 days of culture.

The results were normalized by cell number, determined by nuclei staining (with DAPI: 4′,6-Diamidino-2-Phenylindole, Dihydrochloride), and were represented in percentage of the lysis positive control. Compounds presenting a level of cytotoxicity below 20% compared to control were considered as non-toxic.

### Lipid Accumulation and Lipid Index

After 14 days of culture, preadipocytes have been fixed with 4% paraformaldehyde and then stained by AdipoRed™ at room temperature to reveal the intracellular lipid droplets. Quantification of lipid accumulation has been performed by fluorescence intensity measurements using the spectrophotometer Spark (TECAN).

A second analysis of lipid accumulation was performed with an imaging acquisition and quantification. The area and the intensity of the lipid droplets were evaluated and quantified for more accurate data. An index was calculated (area*intensity of the AdipoRed staining) and normalized by cells number.

### Quantification of Extracellular Secretions

After 13 days, the 24 h culture media of the different conditions have been collected at the end of the treatment period. The concentrations of IL-6, procollagen I and MCP1 have been evaluated by ELISA assays using specific kits (for IL-6: DY206, DuoSet ELISA, R&D Systems; for Procollagen I: DY6220-05, DuoSet ELISA, R&D Systems; for MCP1: DY279-05 DuoSet ELISA, R&D Systems) according to the manufacture's recommendations. Values have been normalized to the cell number determined by DAPI staining.

### Immunofluorescence (Collagen I Network)

One month after fixation of preadipocytes cultivated in pro-inflammatory environment, cells were incubated with 3% Bovine Serum Albumin (BSA) for 30 min in order to block the non-specific sites, then with primary antibody anti-collagen 1 (Novusbio, NB600-408) over-night. After washes with PBS, cells were incubated for 30 min with 3% BSA and then with the secondary antibodies (Goat anti-rabbit alexa-fluor 488, ThermoFisher, A11008) and DAPI (for nuclei staining) for 1 h. After several washes, the acquisition and the quantification were performed with a fluorescent video-microscope. Briefly, the quantification was based on the detection and quantification of cell nuclei stained with DAPI, and the detection of collagen 1 staining. Collagen 1 fibers were detected and measured for their length, thickness and intensity. A Collagen 1 fibers quantity was calculated (Quantity=length*thickness*fluorescence intensity) and normalized by cells number (DAPI staining).

### Results

### Effect of Compound 6 on the Viability of Fibroblasts in Classical Condition

Quantity of LDH released by cells was normalized by cell number in each culture condition. Data are represented in percentage of the positive control. Results are presented in Table 14.

TABLE 14
Cytotoxicity during 24 h after 11 days of culture, normalized
by cell number (in percentage of positive control)

| Conditions | | | Values | | | | Mean | SD |
|---|---|---|---|---|---|---|---|---|
| Positive control | | | 94.5 | 90.8 | 105.9 | 108.8 | 100 | 8.7 |
| Control | | | 23.2 | 25.8 | 21.9 | 18.5 | 22.4 | 3.0 |
| Compound 6 | 2 | mg/ml | 4.2 | 5.1 | 5.3 | 6.0 | 5.1 | 0.8 |
| | 0.5 | mg/ml | 7.1 | 6.6 | 3.1 | 5.7 | 5.6 | 1.8 |
| | 0.1 | mg/ml | 5.9 | 4.8 | 6.5 | 9.8 | 6.7 | 2.1 |
| Compound 44 of | 10 | mg/ml | 54.2 | 50.4 | 40.1 | 38.6 | 45.8 | 7.7 |
| WO2015/140178 | 1 | mg/ml | 18.0 | 18.8 | 19.5 | 18.5 | 18.7 | 0.6 |

No toxicity was observed with compound 6 at 2 mg/ml, 0.5 mg/ml and 0.1 mg/ml. The results showed rather that the LDH release (cytotoxicity) is lower in fibroblasts culture treated with compound 6 (5.1% to 6.7%) compare to the control (22.4%).

In these experimental conditions, compound 6 has a preservative/protective effect on dermal fibroblast even under classical condition with no specific stress.

Compound 6 at all concentrations showed a strong protective effect whereas, in such conditions, Compound 44 of WO2015/140178 does not present any protective effect.

### Effect of Compound 6 on the Proliferation of Preadipocytes in Classical Conditions

The number of cells was determined for each condition by DAPI nuclei staining (nuclei) and expressed in arbitrary unit. Results are presented in Table 15.

TABLE 15
Cells number at the end of the culture,
DAPI staining in Arbitrary Unit (AU)

| | | | | | Standard |
|---|---|---|---|---|---|
| Condition | Values | | | Mean | Deviation |
| Undifferentiation | 8341 | 8042 | 10087 | 8823 | 1105 |
| Differentiation | 11442 | 9029 | 9882 | 10118 | 1224 |
| Compound 6 (2 | 13454 | 12839 | 13298 | 13197 | 320 |
| mg/ml) | | | | | |

The cell number is higher when the cells were treated with the compound 6 at 2 mg/ml (13 197 AU) compared to the differentiated control condition (10 118 AU).

Compound 6 at 2 mg/mL induced a cell proliferation of preadipocytes cultured in classical differentiation conditions.

### Effect of Compound 6 on Human Preadipocytes Under Differentiation Conditions in a Fibro-Inflammation Environment

The effect of compound 6 was evaluated on:
Viability
Proliferation
Lipid accumulation
Extracellular secretions of IL-6, MCP1 and procollagen I
Quantity of Collagen 1 fibers.

### Viability of Preadipocytes in Inflammatory Condition

Quantity of LDH measured in medium was normalized by cell number in each culture condition. Data are represented in percentage of proinflammatory control conditions (AcMC condition). Results are presented in Table 16.

TABLE 16
Cytotoxicity at the end of the culture, normalized
by cell number, in % of AcMC condition

| | | | | | Standard |
|---|---|---|---|---|---|
| Condition | Values | | | Mean | Deviation |
| Undifferentiation | 37.5 | 26.3 | 32.7 | 32.2 | 5.6 |
| AcMC | 122.3 | 88.2 | 89.5 | 100.0 | 19.3 |
| AcMC + compound | 50.5 | 50.6 | 58.9 | 53.3 | 4.8 |
| 6 (2 mg/ml) | | | | | |
| AcMC + compound | 53.1 | 81.8 | 110.4 | 81.7 | 28.7 |
| 6 (1 mg/ml) | | | | | |
| AcMC + Compound | 96.1 | 82.2 | 131.7 | 103.3 | 25.5 |
| 44 of WO2015/ | | | | | |
| 140178 (5 mg/ml) | | | | | |
| AcMC + Compound | 73.7 | 84.9 | 116.0 | 91.5 | 21.9 |
| 44 of WO2015/ | | | | | |
| 140178 (1 mg/ml) | | | | | |

The results showed rather that the LDH release is lower in preadipocyte culture treated with compound 6 (with cytotoxicity of 53.3% and 81.7% respectively at 2 and 1 mg/ml) compared to the control AcMC, i.e. inflammatory conditions, with cytotoxicity fixed at 100%.

In these experimental conditions compound 6 showed a preservative/protective effect on preadipocytes/adipocytes in inflammatory conditions.

A lower relative cytotoxicity was observed with compound 6 at 2 mg/ml (53.3%) and 1 mg/ml (81.7%) compared to Compound 44 of WO2015/140178 at 5 mg/ml (103.3%) and Compound 44 of WO2015/140178 at 1 mg/ml (91.5%). So compound 6 showed a better preservative effect than Compound 44 of WO2015/140178.

### Proliferation of Preadipocytes in Inflammatory Condition

The number of cells was determined for each condition by DAPI nuclei staining (nuclei) and expressed in arbitrary unit. Results are presented in Table 17.

TABLE 17
Cells number at the end of the culture,
DAPI staining in Arbitrary Unit (AU)

| | | | | | Standard |
|---|---|---|---|---|---|
| Condition | Values | | | Mean | Deviation |
| AcMC | 12412 | 13250 | 12795 | 12819 | 420 |
| AcMC + Compound | 20261 | 20369 | 19811 | 20147 | 296 |
| 6 (2 mg/ml) | | | | | |
| AcMC + Compound | 18502 | 17976 | 17985 | 18154 | 301 |
| 6 (1 mg/ml) | | | | | |
| AcMC + Compound | 15556 | 15310 | 14211 | 15026 | 716 |
| 44 of WO2015/ | | | | | |
| 140178 (1 mg/ml) | | | | | |

The cell number is higher when the cells were treated with the compound 6 at 2 mg/ml and 1 mg/ml (20 147AU and 18 154 AU respectively) compared to the AcMC control condition, i.e. inflammatory conditions (12 819AU)

Compound 6 induced a cell proliferation of preadipocytes with a dose-effect at 2 mg/ml and 1 mg/ml.

Compared to Compound 44 of WO2015/140178 at 1 mg/ml, the compound 6 at 1 mg/ml induced a higher proliferation of preadipocytes in inflammatory condition (15 026 versus 18 154 cells respectively).

### Total Lipid Synthesis in Preadipocytes Adipocytes in Inflammatory Condition

Lipid accumulation and Lipid index were evaluated for each condition. Data are represented in percentage of proinflammatory control condition (AcMC condition). Results are presented in Table 18A and 18B.

TABLE 18A
Total Lipid Accumulation (Adipored staining) at
the end of the culture (in % of AcMC condition)

| | | | | | Standard |
|---|---|---|---|---|---|
| Condition | Values | | | Mean | Deviation |
| AcMC | 100.7 | 97.9 | 101.4 | 100.0 | 1.9 |
| AcMC + | 213.8 | 229.4 | 218.6 | 220.6 | 8.0 |
| Dexamethasone 100 nM | | | | | |
| AcMC + compound | 137.2 | 132.3 | 141.4 | 137.0 | 4.5 |
| 6 (2 mg/ml) | | | | | |
| AcMC + Compound | 130.3 | 123.6 | 126.5 | 126.8 | 3.3 |
| 6 (1 mg/ml) | | | | | |
| AcMC + Compound | 120.0 | 118.6 | 120.6 | 119.7 | 1.0 |
| 44 of WO2015/ | | | | | |
| 140178 (2.5 mg/ml) | | | | | |
| AcMC + Compound | 104.7 | 106.6 | 103.1 | 104.8 | 1.8 |
| 44 of WO2015/ | | | | | |
| 140178 (1 mg/ml) | | | | | |

Compound 6 induced an increase lipid synthesis at both concentration in the inflammatory media and performed better than Compound 44 of WO2015/140178 at lower concentration.

As shown in previous results, preadipocytes proliferation lead to an increase of total lipid synthesis in inflammatory condition, clearly underlining the potential of compound 6 for plumping/wrinkle filling effect.

TABLE 18B
Lipid Index (Adipored area * intensity) at the end of the culture,
normalized by cells number (DAPI staining) in % of AcMC condition

| | | | | | Standard |
|---|---|---|---|---|---|
| Condition | Values | | | Mean | Deviation |
| AcMC | 57.2 | 117.4 | 125.4 | 100.0 | 37.3 |
| AcMC + Compound | 286.0 | 338.7 | 269.0 | 297.9 | 36.4 |
| 6 (2 mg/ml) | | | | | |
| AcMC + Compound | 150.8 | 156.6 | 168.4 | 158.6 | 9.0 |
| 6 (1 mg/ml) | | | | | |
| AcMC + Compound | 114.2 | 102.2 | 146.1 | 120.9 | 22.7 |
| 44 of WO2015/ | | | | | |
| 140178 (2.5 mg/ml) | | | | | |
| AcMC + Compound | 137.9 | 150.8 | 146.8 | 145.1 | 6.6 |
| 44 of WO2015/ | | | | | |
| 140178 (1 mg/ml) | | | | | |

For better accuracy, another method was used to quantify the lipids. The lipid index is increased by Compound 6 compared to AcMC control at both concentration in the inflammatory media and performed better than Compound 44 of WO2015/140178. This confirm the potential of Compound 6 for increasing lipid synthesis in inflammatory condition.

### Extracellular Secretions of IL-6 by Preadipocytes in Inflammatory Condition

Quantity of IL-6 secreted in medium was measured and was normalized by cell number in each culture condition. Data are represented in percentage of proinflammatory control condition (AcMC condition). Results are presented in Table 19.

TABLE 19
IL-6 secretion at the end of the culture, normalized by
cells number (DAPI staining) in % of AcMC condition

| | | | | | Standard |
|---|---|---|---|---|---|
| Condition | Values | | | Mean | Deviation |
| AcMC | 85.9 | 101.2 | 113.0 | 100.0 | 13.6 |
| AcMC + Dexamethasone | 21.9 | 21.8 | 24.5 | 22.8 | 1.5 |
| 100 nM | | | | | |
| AcMC + Compound | 25.5 | 26.8 | 31.9 | 28.1 | 3.4 |
| 6 (2 mg/ml) | | | | | |
| AcMC + Compound | 66.1 | 95.1 | 90.8 | 84.0 | 15.6 |
| 6 (1 mg/ml) | | | | | |
| AcMC + Compound 44 of | 64.2 | 55.8 | 79.5 | 66.5 | 12.0 |
| WO2015/140178 (5 mg/ml) | | | | | |
| AcMC + Compound 44 of | 63.9 | 94.4 | 105.2 | 87.8 | 21.4 |
| WO2015/140178 (1 mg/ml) | | | | | |

Compound 6 at 2 mg/ml and 1 mg/ml decreased the IL-6 secretion in preadipocytes/adipocytes (28.1 and 84% IL-6 secreted respectively) compared to the AcMC control condition, i.e. in inflammatory conditions, fixed at 100%.

Moreover the inhibition effect on IL-6 synthesis induced by compound 6 at 2 mg/ml (28.1%) is similar to that observed with the DXM at 100 nM (22.8%) Compound 6 showed a strong anti-inflammatory effect on preadipocytes/adipocytes treated with 2 mg/ml and 1 mg/ml with a dose-effect.

The decrease of IL-6 secretion is better with compound 6 at 2 mg/ml (28% of IL-6 production) than that of Compound 44 of WO2015/140178 at 5 mg/ml (66.5% of IL-6 production) in inflammatory conditions. So compound 6 showed a better anti-inflammatory effect than Compound 44 of WO2015/140178.

### Extracellular Secretions of MCP1 by Preadipocytes in Inflammatory Condition

Quantity of MCP1 secreted in medium was measured and was normalized by cell number in each culture condition. Data are represented in percentage of proinflammatory control condition (AcMC condition). Results are presented in Table 20.

TABLE 20
MCP1 secretion at the end of the culture, normalized by
cells number (DAPI staining) in % of AcMC condition

| | | | | Mean | Standard |
|---|---|---|---|---|---|
| Condition | Values (%) | | | (%) | Deviation |
| Undifferentiation | 1.16 | 4.84 | 5.43 | 4 | 2.3 |
| Differentiation | 29.3 | 39.6 | 39.7 | 36 | 6.0 |
| AcMC | 81.2 | 115.4 | 103.4 | 100 | 17.3 |
| AcMC + Dexamethasone | 82.4 | 106.8 | 97.2 | 95 | 12.3 |
| (100 nM) | | | | | |
| Compound 6 (2 mg/ml) | 62.8 | 65.6 | 67.5 | 65 | 2.4 |
| Compound 6 (1 mg/ml) | 58.8 | 67.5 | 85.6 | 71 | 13.6 |

As expected, the MCP1 secretion was increased in the pro-inflammatory environment (AcMC) compared to the differentiation condition. Dexamethasone had no effect on the MCP1 secretion.

Compound 6 at 2 mg/ml and 1 mg/ml decreased the MCP1 secretion in differentiated preadipocytes (65% and 71% respectively) compared to the AcMC control condition, i.e. in inflammatory conditions, fixed at 100%.

Compound 6 showed an anti-inflammatory effect on preadipocytes treated with 2 mg/ml and 1 mg/ml.

Extracellular secretions of procollagen I by preadipocytes in inflammatory condition Quantity of procollagen secreted in medium was measured and normalized by cell number in each culture condition. Data are represented in percentage of proinflammatory control conditions (AcMC condition). Results are presented in Table 21.

TABLE 21
Procollagen I secretion at the end of the culture, normalized
by cells number (DAPI staining) in % of AcMC condition

| | | | | | Standard |
|---|---|---|---|---|---|
| Condition | Values | | | Mean | Deviation |
| AcMC | 101.8 | 103.5 | 94.7 | 100.0 | 4.6 |
| AcMC + Compound | 47.0 | 40.2 | 51.2 | 46.2 | 5.5 |
| 6 (2 mg/ml) | | | | | |
| AcMC + Compound | 39.3 | 57.7 | 52.7 | 49.9 | 9.5 |
| 6 (1 mg/ml) | | | | | |
| AcMC + Compound 44 of | 72.3 | 60.9 | 75.1 | 69.4 | 7.5 |
| WO2015/140178 (1 mg/ml) | | | | | |

Compound 6 at 2 and 1 mg/ml decreased the Procollagen I secretion (46.2% and 49.9% respectively) compared to the AcMC control condition, i.e. inflammatory conditions, fixed at 100%. AcMC condition is known to increase the secretion of procollagen compared to normal differentiation condition.

There was a stronger decrease of Procollagen I secretion with compound 6 at 1 mg/ml compared to Compound 44 of WO2015/140178 at 1 mg/ml (secretion of 49.9% versus 69.4% respectively) in inflammatory conditions.

### Quantity of Collagen 1 Fibers Produced by Preadipocytes in Inflammatory Condition

Collagen I fibers (fibrillar collagen I) quantity was quantified and the data were normalized by cell number (DAPI staining, quantification of nuclei number). Data are represented in percentage of proinflammatory control conditions (AcMC condition). Results are presented in Table 22.

TABLE 22
Collagen I fibers quantity at the end of the culture, normalized
by cells number (Dapi staining) in % of AcMC condition

| | | | | Mean | Standard |
|---|---|---|---|---|---|
| Condition | Values (%) | | | (%) | Deviation |
| Undifferentiation | / | 49.4 | 41.0 | 45 | 6.0 |
| Differentiation | 78.1 | 164.1 | 111.5 | 118 | 43.3 |
| AcMC | 131.0 | 81.0 | 88.0 | 100 | 27.1 |
| AcMC + Dexamethasone | 397.3 | 390.0 | 384.5 | 391 | 6.4 |
| (100 nM) | | | | | |
| Compound 6 (2 mg/ml) | 193.5 | 230.7 | 213.7 | 213 | 18.6 |
| Compound 6 (1 mg/ml) | 136.7 | 160.5 | 165.3 | 154 | 15.3 |

Compound 6 at 2 and 1 mg/ml increased the Collagen I fibers quantity (213% and 154% respectively) compared to the AcMC control condition, i.e. inflammatory conditions, fixed at 100%.

Compound 6 at 2 and 1 mg/ml induced an increase in Collagen I deposition in the extracellular matrix of the pro-inflammatory environment-cultured preadipocytes.

Compound 6 has matrix remodelling effects that seem to be close to those induced by the anti-inflammatory dexamethasone. The apparent diminution of the Procallagen I observed during the previous study could be explained by its transformation in collagen I fibers.

### 2.4. Evaluation of Effects of Compound 6 on Lipid Synthesis in Normal Human Epidermal Keratinocytes (NHEK)

Normal human epidermal keratinocytes (NHEK) were seeded in 12-well plates and incubated in culture medium for 24 hours. The medium was then replaced by culture medium containing or not (control) the test compounds or the reference (CaCl₂+Vitamin C at 1.5 mM+200 μg/ml respectively) in the presence of the radioactive tracer [¹⁴C]-acetate. Cells were incubated for 48 hours. All experimental conditions were performed in triplicate.

Culture medium was Keratinocyte-SFM supplemented with Epidermal Growth Factor (EGF) 0.25 ng/ml, Pituitary extract (PE) 25 μg/ml and Gentamycin 25 μg/ml. The assay medium was Keratinocyte-SFM supplemented with Gentamycin 25 μg/ml.

At the end of incubation, cells were rinsed with PBS solution and then detached from their support by trypsin treatment. The [¹⁴C]-acetate incorporation was then measured by liquid scintillation (measure of radioactivity). The incorporation is correlated with the total lipid neosynthesis. Results presented in Table 23 are expressed as cpm and % of control.

TABLE 23
Stimulation of the total lipid neosynthesis

| | Basic data | | | | | | | | |
|---|---|---|---|---|---|---|---|---|---|
| | [¹⁴C]- | | | | | | Normalized data | | |
| | Acetate | Mean | sem | % | sem | | % | sem | |
| Treatment | (cpm) | (cpm) | (cpm) | Control | (%) | p⁽¹⁾ | Stimulation | (%) | p⁽¹⁾ |
| Control | 71051 | 71204 | 212 | 100 | 0 | — | 0 | 0 | — |
| | 70938 | | | | | | | | |
| | 71623 | | | | | | | | |
| CaCl₂ | 86977 | 87409 | 1270 | 123 | 2 | *** | 23 | 2 | *** |
| (1.5 mM) + | 89793 | | | | | | | | |
| Vitamine C | 85457 | | | | | | | | |
| (200 μg/ml) | | | | | | | | | |
| Compound 6 | 77267 | 78584 | 687 | 110 | 1 | *** | 10 | 1 | *** |
| (1 mg/ml) | 78905 | | | | | | | | |
| | 79581 | | | | | | | | |
| Compound 6 | 84794 | 86022 | 739 | 121 | 1 | *** | 21 | 1 | *** |
| (3 mg/ml) | 87349 | | | | | | | | |
| | 85922 | | | | | | | | |
| | 86860 | | | | | | | | |

⁽¹⁾Thresholds for statistical significance:
ns: >0.05, Not significant;
* 0.01 to 0.05, Significant;
** 0.001 to 0.01, Very significant;
*** <0.001, Extremely significant

In these experimental conditions the effect of compound 6 on lipid synthesis was similar (at 3 mg/ml) to the reference (CaCl₂ 1.5 mM; Vitamin C 200 μg/ml) with respectively a stimulation of 21% and 23% compared to the control.

Moreover this effect of compound 6 was dose dependent since the stimulation at 1 mg/ml was lower (10% compared to the control).

The compound 6 showed an effect on the stimulation of lipid neosynthesis by normal human epidermal keratinocytes which underlines its potential in restoring the barrier effect of the skin especially for dry or atopic skin and for atopic dermatitis, eczema and psoriasis. In addition this improved lipid synthesis will reduce wrinkles associated with the dryness of the skin.

### 2.5. Evaluation of Effects of Compound 6 on Lipid Peroxidation in Normal Human Epidermal Keratinocytes (NHEK)

Normal human epidermal keratinocytes were seeded in 48-well plates and cultured in culture medium for 24 hours and then in assay medium for a further 24 hours. The medium was then removed and replaced by assay medium containing or not (irradiated control) the test compounds or the reference (BHA—butylated hydroxyanisole, lipid peroxidation inhibitor—at 100 μM) and the cells were pre-incubated for 24 hours. After pre-incubation, the specific fluorescent probe for the measurement of lipid peroxides (C11-fluor) was added and the cells were incubated for 45 minutes. Then, the medium was removed and replaced by assay medium containing or not (irradiated control and test compound conditions) the reference and the cells were irradiated with UVB (+UVA)−300 mJ/cm² (+2.1 J/cm²). The lamp used was a SOL500 Sun Simulator equipped with an H2 filter (Dr. Hbnle. AG). After irradiation, the medium was removed and replaced by assay medium containing or not (irradiated control) the test compounds or the reference and the cells were incubated for 30 minutes before flow cytometry analysis. A non-irradiated control condition was performed in parallel. All experimental conditions were performed in triplicate.

At the end of the incubation, in each well, the cells were trypsinized and transferred into specific tubes for the analysis of C11-fluor fluorescence intensity using a BD FACSVerse™ flow cytometer (acquisition of 2000 to 5000 events per tube). The C11-fluor fluorescent probe is a lipid analogue which integrates cell membranes. As the fluorescence intensity of this probe is decreased with oxidation, it is inversely proportional to the lipid peroxidation. In order to facilitate the result interpretation, the lipid peroxidation was expressed using the value “1/fluorescence intensity” in order to have a direct proportionality between the induction of lipid peroxidation and the values of “% of irradiated control”.

Results presented in Table 24 are expressed as fluorescence intensity and as 00 of protection compared to the control.

TABLE 24
Effect on lipid peroxidation under UV stimulation

| | | Basic data | | | | | | | | | |
|---|---|---|---|---|---|---|---|---|---|---|---|
| | | C11- | C11- | | | | | | | | |
| | | fluor | fluor | | | % | | | Normalized data | | |
| | | GMFI | 1/GMFI | Mean | sem | Irradiated | sem | | % | sem | |
| Treatment | | (AU) | (AU) | (AU) | (AU) | control | (%) | p⁽¹⁾ | Protection | (%) | p⁽¹⁾ |
| Non-irradiated control | | 15217 | 6.6E−05 | 6.9E−05 | 2.0E−06 | 8 | 0 | *** | 100 | 0 | *** |
| | | 14696 | 6.8E−05 | | | | | | | | |
| | | 13762 | 7.3E−05 | | | | | | | | |
| Irradiated | Control | 1260 | 7.9E−04 | 8.5E−04 | 5.3E−05 | 100 | 6 | — | 0 | 7 | — |
| conditions: | | 1234 | 8.1E−04 | | | | | | | | |
| UVB - 300 | | 1041 | 9.6E−04 | | | | | | | | |
| mJ/cm² | BHA (100 μM) | 5266 | 1.90E−04 | 2.7E−04 | 4.27E−05 | 31 | 5 | ** | 75 | 5 | *** |
| (+UVA - | | 3653 | 2.74E−04 | | | | | | | | |
| 2.1 J/cm²) | | 2965 | 3.37E−04 | | | | | | | | |
| | Compound 6 | 2118 | 4.7E−04 | 5.6E−04 | 4.5E−05 | 66 | 5 | * | 37 | 6 | * |
| | (1 mg/ml) | 1705 | 5.9E−04 | | | | | | | | |
| | | 1608 | 6.2E−04 | | | | | | | | |
| | Compound 6 | 2143 | 4.7E−04 | 5.6E−04 | 7.2E−05 | 65 | 8 | * | 38 | 9 | * |
| | (3 mg/ml) | 1970 | 5.1E−04 | | | | | | | | |
| | | 1426 | 7.0E−04 | | | | | | | | |
| | Compound 44 of | 1198 | 8.35E−04 | 9.26E−04 | 4.65E−05 | 108 | 5 | ns | −9 | 6 | ns |
| | WO2015/140178 | 1046 | 9.56E−04 | | | | | | | | |
| | (2.5 mg/ml) | 1013 | 9.87E−04 | | | | | | | | |
| | Compound 44 of | 925 | 1.08E−03 | 1.03E−03 | 2.44E−05 | 121 | 3 | * | −23 | 3 | * |
| | WO2015/140178 | 997 | 1.00E−03 | | | | | | | | |
| | (5 mg/ml) | 986 | 1.01E−03 | | | | | | | | |

⁽¹⁾Threshold for statistical significance:
ns: >0.05, Not significant;
* 0.01 to 0.05, Significant;
** 0.001 to 0.01, Very significant;
*** <0.001, Extremely significant

In these experimental conditions, compound 6 showed a moderate protection on lipid peroxidation of 38% (compared to the control).

The compound 6 showed a protective effect on lipid peroxidation in normal human epidermal keratinocytes stimulated by UVB, which underlines its potential for skin protection and anti-aging. In this particular assay, compound 44 of WO2015/140178 did not have any effect on the protection of lipid peroxidation.

2.6. Evaluation of Effect of Compound 6 on the Protection of Normal Human Dermal Fibroblasts Under UVA Irradiation. Evaluation by MTT Reduction Assay.

The protective effects of compound 6 was assessed in normal human dermal fibroblasts (NHDF). The viability of UVA-irradiated NHDF using a standard MTT reduction assay was tested. Prior to these assays, a preliminary cytotoxicity assay was performed on NHDF, using a standard WST-8 reduction assay and morphological observations with a microscope, in order to determine the concentrations to be tested.

### Materials and Methods

Cell type: NHDF, Bioalternatives reference PF2 used at the 8th passage
Culture conditions: 37° C., 5% CO₂
Culture medium: DMEM supplemented with L-glutamine 2 mM, Penicillin 50 U/ml
   Streptomycin 50 μg/ml, Fetal calf serum (FCS) 10%
Irradiation medium: EBSS supplemented with CaCl₂ 0.2 g/l, MgSO₄ 0.2 g/l
Test compound: the compound 6 (MM=356.3 g/mol) was diluted in culture medium at final concentration of 1.25 and 2.5 mg/ml

### General Experimental Procedure

### Cultures and Treatments

Fibroblasts were seeded in 96-well plates and cultured in culture medium for 24 hours. The medium was then replaced by culture medium containing or not (irradiated control) the test compounds and the cells were pre-incubated for 24 hours. After pre-incubation, the medium was removed and replaced by irradiation medium and the cells were irradiated with 35 J/cm². The lamp used was a SOL500 Sun Simulator equipped with an H1 filter (Dr. Hönle, AG). After irradiation, the medium was removed and replaced by assay medium containing or not (irradiated control) the test compounds and the cells were incubated for 24 hours. A non-irradiated control condition was performed in parallel. All experimental conditions were performed in n=5, except for the control conditions in n=12.

### Evaluation of Cell Viability—MTT Assay

At the end of incubation, the cells were incubated with MTT (tetrazolium salt) reduced in blue formazan crystals by succinate dehydrogenase (mitochondrial enzyme). This transformation is proportional to the enzyme activity. After cell dissociation and formazan crystal solubilization using DMSO, the optical density (OD) of the extracts at 540 nm, proportional to the number of living cells and their metabolic activity, was recorded with a microplate reader (VERSAmax, Molecular Devices).

### Data Management

Raw data were analyzed using Microsoft Excel© software. The inter-group comparisons were performed by an unpaired Student's t-test.

The standard error of the mean (sem) is a measure of how far the sample mean is likely to be from the true population mean. The sem is calculated as the standard deviation (sd) divided by the square root of sample size (n). Standard error of the mean: sem=sd/√n Percentage of viability: viability (%)=(OD sample/OD control)×100

### Results

The cell viability (OD 540 nm) from irradiated culture added with tested compound was compared with irradiated control culture and the percentage of viability was calculated. The results are presented in the following Table 25.

TABLE 25
Preservative effect of compound 6 on
NHDF under UVA stimulation (35 J/cm²)

| | | Mean | | % | | |
|---|---|---|---|---|---|---|
| | | OD | | Irradiated | | |
| Conditions | | (540 nm) | sem | control | sem | p⁽¹⁾ |
| Non-irradiated control | | 1.02 | 0.01 | 918 | 9 | *** |
| Irradiated Control | | 0.11 | 0.01 | 100 | 7 | — |
| Compound 6 | 1.25 mg/ml | 0.13 | 0.02 | 116 | 14 | ns |
| | 2.5 mg/ml | 0.15 | 0.01 | 130 | 12 | * |

⁽¹⁾: Threshold for statistical significance
ns: >0.05, Not significant
* 0.01 to 0.05, Significant
** 0.001 to 0.01, Very significant
*** <0.001, Extremely significant

When tested at 2.5 mg/ml on irradiated cells, the compound 6 induced a significant increase of cell viability (130% of the irradiated control). The compound 6 displayed a statistically significant protective effect against UVA irradiation.

### 2.7. Evaluation of Effect of the Compound 6 on a Coculture of Human Aged Fibroblasts and Mature Adipocytes.

In order to evaluate its effect on fibroblast matrix and its anti-inflammatory properties, the effect of compound 6 has been tested on an adipocytes-aged fibroblasts coculture model, mimicking the interactions between dermis and hypodermis in the skin and allowed to explore in parallel the biological effects of the products on two cellular targets.

### Materials and Methods

Experimental model: co-culture of human mature adipocytes cultured in 3D and human dermal fibroblasts cultured in 2D (AM3D-FB2D co-culture). This experimental model of AM3D-FB2D co-culture mimicked the interactions between dermis and hypodermis in the skin and allowed to explore in parallel the biological effects of the products on two cellular targets.
Donor characteristics: This study was performed on mature adipocytes and fibroblasts from two different donors (one donor for mature adipocytes and another donor for dermal fibroblasts).
   Fibroblasts donor=woman of 56-year-old, BMI=28 kg/m²
   Adipocytes donor=woman of 27-year-old, BMI=21.4 kg/m²
Treatment procedure: compound 6 was added to the co-culture medium daily for up to 6 days

### General Experimental Procedure

### Cultures and Treatments

Dermal fibroblasts were seeded in DMEM 10% FBS at 10000 cells/well. The day after, the adipocytes capsules of 50 μl were added in suspension above the fibroblasts and the medium was changed and replaced by a specific culture medium for the co-culture AM3D-FB2D. The formation of adipocytes capsules followed an internal standardized protocol. Briefly, the fully mature adipocytes were isolated from the hypodermis after digestion by collagenase. The isolated adipocytes were then washed with a wash buffer and encapsulated in a peptidic hydrogel to form 3D adipocytes capsules of 50 μl in size. Cells were incubated at 37° C. and 5% CO₂ overnight for stabilization. Treatments were initiated at DO with a medium change at DO, D2, D3 and D5. The entire culture media of 24 h incubation was collected at D3 and D6, before being centrifugated and stored at −80° C. Each culture condition was done in triplicate.

### Biochemical Analyses

The biochemical analyses on culture media were performed via ELISA using specific kits according to the manufacturer's recommendations: IL-6 (Duoset DY206, R&D Systems), and Hyaluronic Acid (HA) (Duoset, DY3614-05, R&D Systems and Procollagen I (Duoset, DY6220-05, R&D Systems) The results of HA and Procollagen I were normalized by fibroblasts cell number regarding the fact that these molecules were secreted mainly by fibroblasts. All the biochemical results were represented in percentage of the control condition.

### Results

### Interleukin 6 Secretion

To evaluate the effect of the compound 6 on inflammation, the extracellular concentrations of IL-6, that is secreted by the fibroblasts but mainly by the adipocytes, were measured at D3 and D6. The results are presented in the following Table 26.

TABLE 26
secretion of IL-6 in percentage of the coculture control
condition, after 3 and 6 days of treatment

| | | Values | | | Mean | Standard |
|---|---|---|---|---|---|---|
| | Conditions | (%) | | | (%) | Deviation |
| D3 | Control | 101 | 82 | 117 | 100 | 17 |
| | Dexamethasone 100 nM | 22 | 48 | 31 | 34 | 13 |
| | Compound 6 (3 mg/ml) | 75 | 63 | 79 | 73 | 8 |
| | Compound 6 (2 mg/ml) | 56 | 78 | 82 | 72 | 14 |
| D6 | Control | 121 | 82 | 97 | 100 | 20 |
| | Dexamethasone 100 nM | 39 | 24 | 61 | 41 | 19 |
| | Compound 6 (3 mg/ml) | 59 | 54 | 71 | 61 | 8 |
| | Compound 6 (2 mg/ml) | 46 | 66 | 45 | 53 | 12 |

The anti-inflammatory reference item, the dexamethasone, reduced the secretion of this pro-inflammatory cytokine to 34% and 41% compared to the control condition (100%) after 3 and 6 days of treatment respectively (Table 26). The compound 6 induced also an IL-6 decrease, at 3 mg/ml and 2 mg/ml, up to 73 and 72% at D3 and 61% and 53% at D6 respectively

These results showed that the compound 6 has an anti-inflammatory effect on a co-culture of aged fibroblasts and matures adipocytes which underlines its potential for the treatment of inflammaging.

### Hyaluronic Acid Secretion

To evaluate the effect of the compound 6 on fibroblast's matrix, the extracellular concentrations of Hyaluronic Acid (HA), that is secreted only by the fibroblasts, were measured at D3 and D6. The results are presented in the following Table 27.

TABLE 27
secretion of HA, normalized by fibroblasts number, in percentage of
the coculture control condition, after 3 and 6 days of treatment

| | | Values | | | Mean | Standard |
|---|---|---|---|---|---|---|
| | Conditions | (%) | | | (%) | Deviation |
| D3 | Control | 110 | 79 | 110 | 100 | 18 |
| | Compound 6 (3 mg/ml) | 83 | 75 | 98 | 85 | 12 |
| | Compound 6 (2 mg/ml) | 96 | 93 | 80 | 90 | 9 |
| | Compound 6 (1 mg/ml) | 127 | 123 | 169 | 140 | 26 |
| D6 | Control | 81 | 107 | 113 | 100 | 17 |
| | Compound 6 (3 mg/ml) | 121 | 213 | 159 | 164 | 46 |
| | Compound 6 (2 mg/ml) | 240 | 233 | 203 | 225 | 20 |
| | Compound 6 (1 mg/ml) | 172 | 157 | 237 | 189 | 43 |

After 3 days of treatment, the compound 6 induced an increase in HA secretion (140% of the control) at the lowest concentration of 1 mg/ml. After 6 days of treatment, the compound 6 induced great increases in HA secretion in the three tested conditions, i.e. 164%, 225% and 189% of the control at concentrations of 3, 2 and 1 mg/ml respectively. These results showed that the compound 6 has an effect on the extra cellular matrix of aged fibroblasts by increasing production of HA that play a crucial role in skin moisturizing, and skin plumping.

### Procollagen I Secretion

To evaluate the effect of the compound 6 on fibroblasts' matrix, the extracellular concentrations of Procollagen I, that is secreted only by the fibroblasts, were measured at D3 and D6. The results are presented in the following Table 28.

TABLE 28
secretion of Procollagen I, normalized by fibroblasts
number, in percentage of the coculture control
condition, after 3 and 6 days of treatment

| | | | | | | Standard |
|---|---|---|---|---|---|---|
| | Conditions | Values | | | Mean | Deviation |
| D3 | Control | 77 | 128 | 95 | 100 | 26 |
| | Positive control 10% FBS | 389 | 344 | 724 | 486 | 208 |
| | Compound 6 (3 mg/ml) | 202 | 78 | 238 | 173 | 84 |
| | Compound 6 (2 mg/ml) | 177 | 144 | 157 | 159 | 17 |
| | Compound 6 (1 mg/ml) | 162 | 158 | 168 | 163 | 5 |
| D6 | Control | 121 | 89 | 89 | 100 | 18 |
| | Positive control 10% FBS | 240 | 323 | 723 | 428 | 258 |
| | Compound 6 (3 mg/ml) | 515 | 398 | 508 | 474 | 66 |
| | Compound 6 (2 mg/ml) | 284 | 488 | 460 | 411 | 111 |
| | Compound 6 (1 mg/ml) | 312 | 472 | 469 | 418 | 92 |

After 3 days of treatment, compound 6 induced a slight increase of the Procollagen I secretion at all the concentrations, i.e. 173%, 159% and 163% of the control at concentration of 3, 2 and 1 mg/ml respectively.

After 6 days of treatment, compound 6 increased the Procollagen I secretion in a higher extend at all the concentrations, i.e. 474%, 411% and 418% of the control at concentration of 3, 2 and 1 mg/ml respectively.

This effect at D6 is close or higher than the positive control (428%).

These results showed that the compound 6 has an effect on the extra cellular matrix of aged fibroblasts by increasing production Procollagen I that play a crucial role in skin anti-aging.

1. A compound of formula (I): 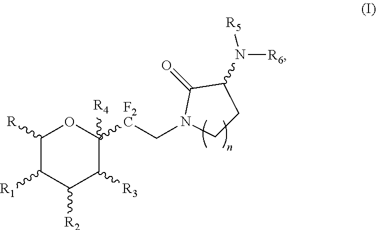
or a salt thereof, a solvate, a tautomer, a stereoisomer or a mixture of stereoisomers in any proportion,
wherein:
n represents 1 or 2,
R represents CH₂OR₈,
R₁ and R₂ represent, independently from one another, OR₁₅,
R₃ represents OR₂₂,
R₄ represents a hydrogen or halogen atom or an OSiR^{a4}R^{b4}R^{c4}, OR₄₁, OC(O)R₄₂, OCO₂R₄₃, OCONR₄₄R₄₅, OP(O)(OR₄₆)₂, or OSO₃R₄₇ group,
or R and R₁, together with the carbon atoms to which they are attached, form a cyclic acetal having the formula: 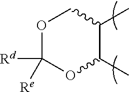
and/or (R₁ and R₂), (R₂ and R₃), and/or (R₃ and R₄), together with the carbon atoms to which they are attached, form a cyclic acetal having the formula: 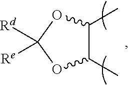
R₅ and R₆, identical or different, represent a hydrogen atom or a N-protecting group,
R₈, R₁₅, R₂₂ and R₄₁ represent, independently from one another, a hydrogen atom, a O-protecting group or a (C₁-C₆)alkyl, (C₂-C₆)alkenyl, (C₂-C₆)alkynyl, (C₃-C₇)cycloalkyl, 5- to 7-membered heterocycloalkyl, aryl, heteroaryl, aryl-(C₁-C₆)alkyl, heteroaryl-(C₁-C₆)alkyl, (C₁-C₆)-alkyl-aryl, (C₁-C₆)-alkyl-heteroaryl, saccharidic or polysaccharidic group which is optionally substituted with one or more groups selected from the group consisting of a halogen atom, (C₁-C₆)alkoxy, OH, COOH and CHO,
R₄₂ and R₄₃ represent, independently from one another, a (C₁-C₆)alkyl, (C₂-C₆)alkenyl, (C₂-C₆)alkynyl, (C₃-C₇)cycloalkyl, 5- to 7-membered heterocycloalkyl, aryl, heteroaryl, aryl-(C₁-C₆)alkyl, heteroaryl-(C₁-C₆)alkyl, (C₁-C₆)-alkyl-aryl or (C₁-C₆)-alkyl-heteroaryl group which is optionally substituted with one or more groups selected from the group consisting of a halogen atom, (C₁-C₆)alkoxy, OH, COOH and CHO,
R₄₄ and R₄₅ represent, independently from one another, a hydrogen atom or a (C₁-C₆)alkyl, (C₂-C₆)alkenyl, (C₂-C₆)alkynyl, aryl, heteroaryl, aryl-(C₁-C₆)alkyl, heteroaryl-(C₁-C₆)alkyl, (C₁-C₆)-alkyl-aryl or (C₁-C₆)-alkyl-heteroaryl group which is optionally substituted with one or more groups selected from the group consisting of a halogen atom, (C₁-C₆)alkoxy, OH, COOH and CHO,
R₄₆ and R₄₇ represent, independently from one another, a hydrogen atom or a (C₁-C₆)alkyl group,
R^{a4}, R^{b4} and R^{c4} represent, independently from one another, a (C₁-C₆)alkyl, aryl or aryl-(C₁-C₆)alkyl group, and
R^{d} and R^{e} represent, independently from one another, a hydrogen atom or a (C₁-C₆)alkyl group.

2. The compound according to claim 1, which is a compound of formula (Ia), (Ib), (Ic) or (Id): 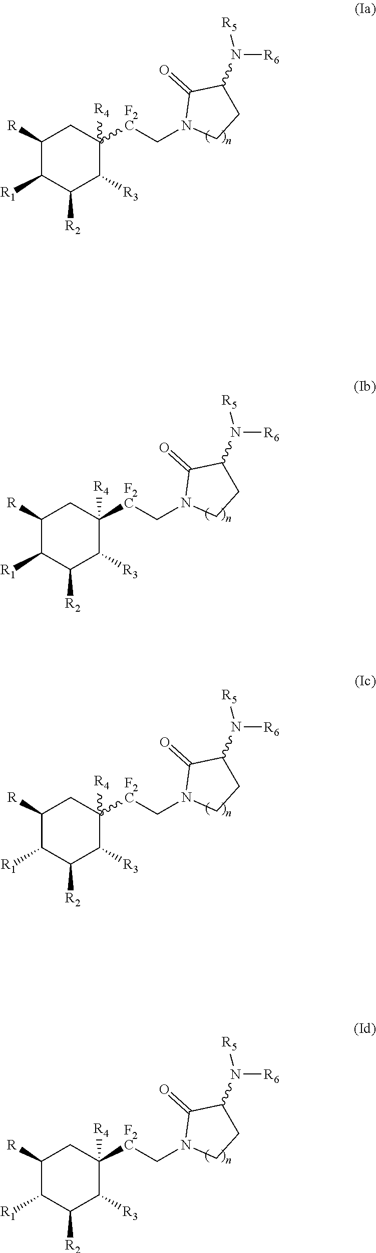
or a salt thereof, a solvate, a tautomer, a stereoisomer or a mixture of stereoisomers in any proportion.

3. The compound according to claim 1, wherein R₄ is a hydrogen atom or OR₄₁.

4. The compound according to claim 1, wherein R is CH₂OH or CH₂OBn; R₁ and R₂ are, independently from one another, OH or OBn; R₃ is OH or OBn; and R₄ is H, OH or OBn.

5. The compound according to claim 1, wherein R₁, R₂ and R₃ are identical.

6. The compound according to a claim 1, wherein R₅ and R₆, identical or different, are a hydrogen atom or a —CO₂—R_{GP1} group with R_{GP1} representing a (C₁-C₆)alkyl optionally substituted with one or several halogen atoms; a (C₂-C₆)alkenyl; an aryl optionally substituted with one or several groups selected from the group consisting of a methoxy group and a nitro group; an aryl-(C₁-C₆)alkyl, the aryl moiety which is optionally substituted with one or several methoxy groups; or a 9-fluorenylmethyl group.

7. The compound according to claim 1, which is chosen among the compounds: 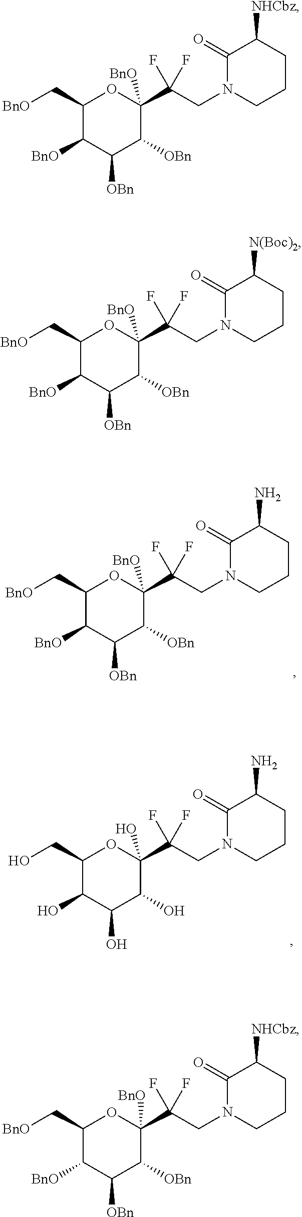 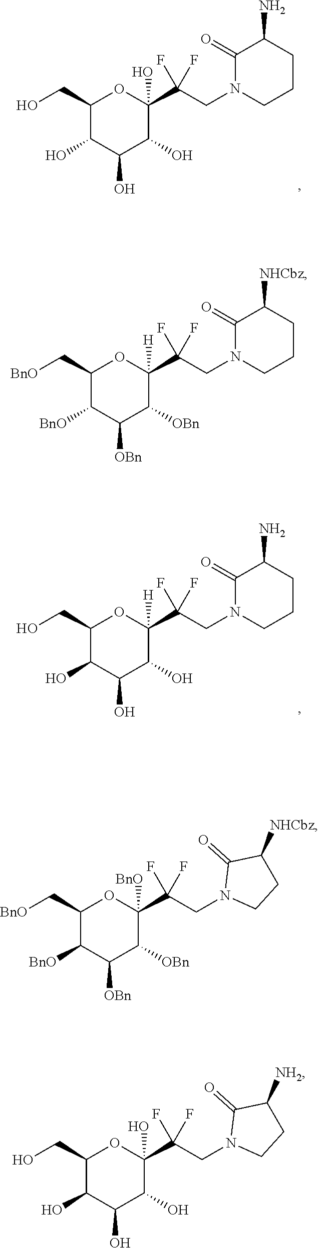 [SMILES: N[C@H]1CCCN(CC(F)(F)[C@]2(O)O[C@H](CO)[C@@H](O)[C@H](O)[C@H]2O)C1=O ; O=C(N[C@H]1CCCN(CC(F)(F)[C@@H]2O[C@H](COCc3ccccc3)[C@@H](OCc3ccccc3)[C@H](OCc3ccccc3)[C@H]2OCc2ccccc2)C1=O)OCc1ccccc1 ; N[C@H]1CCCN(CC(F)(F)[C@@H]2O[C@H](CO)[C@H](O)[C@H](O)[C@H]2O)C1=O ; O=C(N[C@H]1CCN(CC(F)(F)[C@]2(OCc3ccccc3)O[C@H](COCc3ccccc3)[C@H](OCc3ccccc3)[C@H](OCc3ccccc3)[C@H]2OCc2ccccc2)C1=O)OCc1ccccc1 ; N[C@H]1CCN(CC(F)(F)[C@]2(O)O[C@H](CO)[C@H](O)[C@H](O)[C@H]2O)C1=O]
and the salts and solvates thereof.

8. A process for preparing the compound according to claim 1 comprising:
(a) cyclizing a compound of the formula (II): 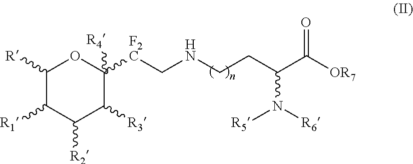
wherein:
n is as defined in formula I,
R′, R₁′, R₂′, R₃′, R₄′, R₅′ and R₆′ correspond respectively to R, R₁, R₂, R₃, R₄, R₅ and R₆ as defined in formula I, optionally in a protected form, and
R₇ represents a (C₁-C₆)alkyl or an aryl-(C₁-C₆)alkyl,
to obtain a compound of formula (I) optionally in a protected form,
(b) when R′, R₁′, R₂′, R₃′, R₄′, R₅′ and/or R₆′ represent a protected form of R, R₁, R₂, R₃, R₄, R₅ and/or R₆ respectively, deprotecting the protected form of R, R₁, R₂, R₃, R₄, R₅ and/or R₆ to obtain a compound of formula (I), and
(c) optionally salifying or solvating the compound obtained in previous step (a) or (b) to obtain a salt or solvate of a compound of formula (I).

9. A cosmetic or pharmaceutical composition comprising a compound according to claim 1 and a physiologically acceptable excipient.

10-15. (canceled)

16. A dressing comprising a pad, compress or sponge impregnated with a cosmetic or pharmaceutical composition according to claim 9.

17. (canceled)

18. A culture, storage and/or preservation medium comprising a compound according to claim 1.

19. The compound according to claim 1, wherein:
R₈, R₁₅, R₂₂ and R₄₁ represent, independently from one another, a hydrogen atom or a (C₁-C₆)alkyl, aryl, aryl-(C₁-C₆)alkyl, saccharidic or polysaccharidic group which is optionally substituted with one or more groups selected from the group consisting of a halogen atom, (C₁-C₆)alkoxy, OH, COOH and CHO,
R₄₂ and R₄₃ represent, independently from one another, a (C₁-C₆)alkyl, aryl or aryl-(C₁-C₆)alkyl group which is optionally substituted with one or more groups selected from the group consisting of a halogen atom, (C₁-C₆)alkoxy, OH, COOH and CHO, and
R₄₄ and R₄₅ represent, independently from one another, a hydrogen atom or a (C₁-C₆)alkyl, (C₂-C₆)alkenyl, (C₂-C₆)alkynyl, aryl-(C₁-C₆)alkyl, heteroaryl-(C₁-C₆)alkyl group which is optionally substituted with one or more groups selected from the group consisting of a halogen atom, (C₁-C₆)alkoxy, OH, COOH and CHO.

20. The compound according to claim 19, wherein:
R₈, R₁₅, R₂₂ and R₄₁ represent, independently from one another, a hydrogen atom, or a (C₁-C₆)alkyl, aryl or aryl-(C₁-C₆)alkyl group which is optionally substituted with one or more groups selected from the group consisting of a halogen atom, (C₁-C₆)alkoxy, OH, COOH and CHO, and
R₄₄ and R₄₅ represent, independently from one another, a hydrogen atom or a (C₁-C₆)alkyl, aryl or aryl-(C₁-C₆)alkyl group which is optionally substituted with one or more groups selected from the group consisting of a halogen atom, (C₁-C₆)alkoxy, OH, COOH and CHO.

21. The compound according to claim 1, wherein R₅ and R₆, identical or different, are H, benzyloxycarbonyl (Cbz) or tbutyloxycarbonyl (Boc).

22. The compound according to claim 7, wherein the acid addition salts are acid addition salts with hydrochloric acid or acetic acid.

23. A method for skin plumping and/or skin volumizing and/or skin densifying and/or wrinkle filling and/or skin or hair moisturizing and/or skin or hair relipiding and/or stimulating hair growth comprising administrating to a person in need thereof an effective amount of the compound according to claim 1 or a cosmetic or pharmaceutical composition comprising the compound according to claim 1 and a physiologically acceptable excipient.

24. A method for treating or preventing skin aging, for protecting skin or for regenerating skin comprising applying to a skin the compound according to claim 1 or a cosmetic or pharmaceutical composition comprising the compound according to claim 1 and a physiologically acceptable excipient.

25. A method for treating dry skin and/or atopic dermatitis and/or atopic eczema and/or psoriasis, for treating inflammation, for treating or preventing a fibrosis disease, or for healing comprising administrating to a person in need thereof an effective amount of a compound according to claim 1 or a cosmetic or pharmaceutical composition comprising a compound according to claim 1 and a physiologically acceptable excipient.

26. The method according to claim 25, wherein the inflammation is a chronic, low-grade inflammation; and the fibrosis disease is an excessive scar.

27. The method according to claim 26, wherein the excessive scar is a keloid or a hypertrophic scar.

28. The method according to claim 25, wherein the method is for treating or preventing a fibrosis disease, or for healing, and wherein the compound according to formula I or the cosmetic or pharmaceutical composition is administered topically in combination with a laser or surgical treatment.

29. A method for preserving and/or protecting a biological material or a microorganism comprising placing the biological material or the microorganism in a medium containing the compound according to claim 1.

30. The method according to claim 29, wherein the biological material is cells, a tissue, a body fluid or an organ.
